(19) United States
(12) Patent Application Publication
(10) Pub. No.: US 2018/0303807 A1
(43) Pub. Date: Oct. 25, 2018

(54) ANDROGEN RECEPTOR LIGANDS

(71) Applicants: John KATZENELLENBOGEN, Carbondale, IL (US); Jatinder JOSAN, Blacksburg, VA (US); DUKE UNIVERSITY, Durham, NC (US); BOARD OF TRUSTEES OF SOUTHERN ILLINOIS UNIVERSITY, Carbondale, IL (US); VIRGINIA POLYTECHNIC INSTITUTE AND STATE UNIVERSITY, Blacksburg, VA (US)

(72) Inventors: John A. Katzenellenbogen, Urbana, IL (US); Jatinder Josan, Blacksburg, VA (US); John Norris, Raleigh, NC (US); Donald P. McDonnell, Chapel Hill, NC (US)

(21) Appl. No.: 15/765,140

(22) PCT Filed: Oct. 1, 2016
(86) PCT No.: PCT/US16/55085

§ 371 Date: Mar. 30, 2018

Related U.S. Application Data
(60) Provisional application No. 62/235,699, filed on Oct. 1, 2015.

(51) Int. Cl. A61K 31/4375; A61P 35/00; A61K 31/4365

(52) U.S. Cl. CPC A61K 31/4375; A61K 31/4365; A61P 35/00

(57) ABSTRACT

Described are compounds that inhibit androgen receptor action, pharmaceutical compositions including the compounds, and methods of using the compounds and compositions for treating disorders and conditions in a subject.

### CROSS REFERENCE TO RELATED APPLICATION

This application claims priority to U.S. Provisional Application No. 62/235,699, filed Oct. 1, 2015, which is herein incorporated by reference in its entirety.

### STATEMENT OF GOVERNMENT INTEREST

This invention was made with government support under Grant No. N00014-14-P-1137, awarded by the National Institutes of Health; and Grant No. N00014-15-P-0158, awarded by the Department of Defense. The government has certain rights in the invention.

### TECHNICAL FIELD

The present disclosure is concerned with compounds having activity on androgen receptors (e.g., activity as an agonist, partial agonist, inhibitor and/or degradation activity against androgen receptor). The compounds may be selective androgen receptor modulators (SARMs) or selective androgen receptor degraders (SARDs). The present disclosure further concerns compositions comprising these compounds as active ingredients as well as processes for preparing these compounds and compositions.

### BACKGROUND

The androgen receptor (AR) plays an integral role in primary and secondary male sexual development. While abnormalities resulting in an attenuation of the AR response to endogenous hormones (testosterone and its reduced form, 5α-dihydrotestosterone or DHT) produce male infertility and feminization, excessive stimulation of AR can also result in pathologies. The most commonly presented diseases of this type are prostate cancer and the related, but benign, prostatic hyperplasia. Both of these diseases are responsive to endocrine-based treatments that attempt to suppress tumor/prostate growth either by direct administration of an AR antagonist or by ‘chemical castration’ techniques that result in decreased gonadal production of the endogenous agonist, testosterone.

Although current clinically-applied anti-androgen therapies lead to disease regression, eventually all patients develop resistance to these drugs over the course of months to a few years. At this point the cancer will continue to progress despite administration of the compound, now termed as castration-resistant prostate cancer (CRPC). CRPC is associated with increased levels of AR action. First generation anti-androgens such as bicalutamide display agonistic properties in cells overexpressing AR, or in cells harboring W741 C mutation. Hydroflutamide displays similar agonistic properties in cell line harboring T877A mutation (generally referred to as LNCaP cells). In vitro and in vivo, increased AR expression has been shown to confer resistance to anti-androgen therapy. To overcome some of these problems, second generation antiandrogens such as the recently approved enzalutamide (formerly known as MDV3100) have shown some promise; although resistance to this drug has also been encountered in its short time on the market. To address these issues, third generation antiandrogens that retain antagonism in cells expressing excess AR, or cells with the commonly encountered AR mutations, and that degrade the androgen receptor, thus, depleting its cellular pools, are expected to have utility in the treatment of CRPC.

### SUMMARY

In one aspect, disclosed is a compound of formula (I), or a pharmaceutically acceptable salt thereof,

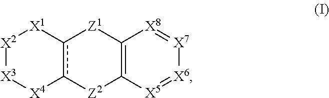

wherein X¹ is C(R^{1a}R^{1b}), S, C(O), C(S), S(O), or S(O)₂, or N—R^{1c}; X² is a bond, C(R^{2a}R^{2b}), or N—R^{2c}; X³ is a bond, C(R^{3a}R^{3b}), or N—R^{3c}; X⁴ is a bond, C(R^{4a}R^{4b}), C(R^{4a}R^{4b})—C(R^{4a}R^{4b}), C(O), C(S), S(O), S(O)₂, or N—R^{4c}; provided that no more than two of X²-X⁴ are simultaneously a bond; R^{1a}, R^{1b}, R^{2a}, R^{2b}, R^{3a}, R^{3b}, R^{4a}, and R^{4b} are each independently selected from the group consisting of hydrogen, deuterium, halogen, alkyl, alkenyl, alkynyl, heteroalkyl, aryl, heteroaryl, cycloalkyl, heterocycloalkyl, and —SO₂-alkyl; R^{1c}, R^{2c}, R^{3c}, and R^{4c} are each independently selected from the group consisting of hydrogen, deuterium, and alkyl; optionally R^{1a} and R^{1b}, R^{2a} and R^{2b}, R^{3a} and R^{3b}, R^{4a} and R^{4b}, R^{1a} and R^{2a}, R^{2a} and R^{3a}, R^{3a} and R^{4a}, R^{1c} and R^{2a}, R^{1c} and R^{2c}, R^{1a} and R^{2c}, R^{2c} and R^{3a}, R^{2c} and R^{3c}, R^{2a} and R^{3c}, R^{3c} and R^{4a}, R^{3c} and R^{4c}, or R^{3a} and R^{4c} together with the carbon atoms to which they are attached form an aryl, heteroaryl, cycloalkyl, cycloalkenyl, or heterocycloalkyl; X⁵ is C(R⁵), N, or a bond; X⁶ is C(R⁶) or N; X⁷ is C(R⁷) or N; X⁸ is C(R⁸) or N; Z¹ is C(R⁹R^{9′}); Z² is N—R¹⁰, C(R^{10′}R^{10″}), O, or S; R⁵, R⁶, R⁷, R⁸, R⁹, R^{10′}, and R^{10″} are each independently selected from the group consisting of hydrogen, deuterium, halogen, alkyl, alkenyl, alkynyl, heteroalkyl, aryl, heteroaryl, cycloalkyl, heterocycloalkyl, cyano, nitro, hydroxy, alkoxy, amino, alkylamino, and dialkylamino; R^{9′} is hydrogen or deuterium; optionally R⁵ and R⁶, R⁶ and R⁷, or R⁷ and R⁸ together with the carbon atoms to which they are attached form an aryl, heteroaryl, cycloalkyl, cycloalkenyl, or heterocycloalkyl; R¹⁰ is selected from the group consisting of hydrogen, deuterium, and alkyl; wherein said alkyl, alkenyl, alkynyl, heteroalkyl, alkoxy, aryl, heteroaryl, cycloalkyl, cycloalkenyl, and heterocycloalkyl, at each occurrence, whether alone or part of another group, are independently substituted with 0, 1, 2, 3, 4, or 5 substituents independently selected from the group consisting of deuterium, halogen, oxo (═O), ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycloalkyl, cycloalkylalkyl, cycloalkenylalkyl, heteroarylalkyl, arylalkyl, heterocycloalkylalkyl, hydroxy, hydroxyalkyl, alkoxy, alkylthio, alkoxyalkyl, alkylene, aryloxy, arylthio, phenoxy, benzyloxy, amino, alkylamino, dialkylamino, acylamino, aminoalkyl, arylamino, diarylamino, sulfonylamino, sulfinylamino, sulfonyl, alkylsulfonyl, arylsulfonyl, aminosulfonyl, sulfinyl, —COOH, ketone, alkoxycarbonyl, aryloxycarbonyl, amide, carbamate, acyl, boronic acid, and boronic ester.

In certain embodiments, the compounds of formula (I) have formula (I′):

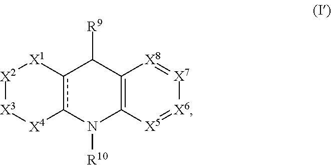

or a pharmaceutically acceptable salt thereof, wherein X¹ is C(R^{1a}R^{1b}), C(O), C(S), S(O), or S(O)₂; X² is a bond or C(R^{2a}R^{2b}); X³ is C(R^{3a}R^{3b}); X⁴ is C(R^{4a}R^{4b}), C(O), C(S), S(O), or S(O)₂; R^{1a}, R^{1b}, R^{2a}, R^{2b}, R^{3a}, R^{3b}, R^{4a}, and R^{4b} are each independently selected from the group consisting of hydrogen, halogen, alkyl, alkenyl, alkynyl, aryl, heteroaryl, cycloalkyl, heterocycloalkyl, and —SO₂-alkyl; X⁵ is C(R⁵) or N; X⁶ is C(R⁶) or N; X⁷ is C(R⁷) or N; X⁸ is C(R⁸) or N; R⁵, R⁶, R⁷, and R⁸ are each independently selected from the group consisting of hydrogen, halogen, alkyl, alkenyl, alkynyl, aryl, heteroaryl, cycloalkyl, heterocycloalkyl, cyano, nitro, hydroxy, alkoxy, amino, alkylamino, and dialkylamino; optionally R⁵ and R⁶, R⁶ and R⁷, or R⁷ and R⁸ together with the carbon atoms to which they are attached form a 5- or 6-membered aryl, heteroaryl, cycloalkyl, cycloalkenyl, or heterocycloalkyl; R⁹ is aryl, heteroaryl, cycloalkyl, or heterocycloalkyl; and R¹⁰ is hydrogen or alkyl; wherein said alkyl, alkenyl, alkynyl, alkoxy, aryl, heteroaryl, cycloalkyl, and heterocycloalkyl, at each occurrence, whether alone or part of another group, are independently substituted with 0, 1, 2, 3, 4, or 5 substituents independently selected from the group consisting of halogen, oxo (═O), ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycloalkyl, cycloalkylalkyl, cycloalkenylalkyl, heteroarylalkyl, arylalkyl, heterocycloalkylalkyl, hydroxy, hydroxyalkyl, alkoxy, alkylthio, alkoxyalkyl, alkylene, aryloxy, arylthio, phenoxy, benzyloxy, amino, alkylamino, dialkylamino, acylamino, aminoalkyl, arylamino, diarylamino, sulfonylamino, sulfinylamino, sulfonyl, alkylsulfonyl, arylsulfonyl, aminosulfonyl, sulfinyl, —COOH, ketone, alkoxycarbonyl, aryloxycarbonyl, amide, carbamate, acyl, boronic acid, and boronic ester.

Also disclosed are pharmaceutical compositions comprising the compounds, methods of making the compounds, and methods of using the compounds.

### DETAILED DESCRIPTION

Disclosed herein are compounds with a ring-fused 1,4-dihydropyridine (DHP) core and analogs thereof. The compounds can be used for treating hormone refractory prostate cancer, especially under cellular conditions that are resistant to treatment using current clinically-available antiandrogens (e.g., flutamide, bicalutamide, nilutamide, and cyroproterone acetate). Also disclosed are pharmaceutical compositions comprising the compounds and methods of making the compounds. Also disclosed are methods of using the compounds for treating cancer, tumor growth, metastatic growth, fibrosis and the like, comprising administration of a therapeutically effective amount of a compound disclosed herein, or a pharmaceutically acceptable salt thereof to a subject in need thereof.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definition of standard chemistry terms may be found in reference works, including Carey and Sundberg “ADVANCED ORGANIC CHEMISTRY 4TH ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. In this application, the use of “or” means “and/or” unless stated otherwise. Furthermore, use of the term “including” as well as other forms, such as “include”, “includes,” and “included,” is not limiting.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain containing from 1 to 10 carbon atoms. The term “lower alkyl” or “C₁-C₆-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety.

The term “alkenyl,” as used herein, means a straight or branched, hydrocarbon chain containing at least one carbon-carbon double bond and from 1 to 10 carbon atoms.

The term “alkoxyalkyl,” as used herein, refers to an alkoxy group, as defined herein, appended to the parent molecular moiety through an alkyl group, as defined herein.

The term “alkylene,” as used herein, refers to a divalent group derived from a straight or branched chain hydrocarbon of 1 to 10 carbon atoms, for example, of 2 to 5 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

The term “alkylamine,” as used herein, refers to —N(alkyl)ₓH_{y} group, where x and y are selected from the group x=1, y=1 and x=2, y=0. When x=2, the alkyl groups, taken together, can optionally form a cyclic ring system.

The term “alkynyl,” as used herein, refers to a type of alkyl group in which the first two atoms of the alkyl group form a triple bond. That is, an alkynyl group begins with the atoms —C═C—R, wherein R refers to the remaining portions of the alkynyl group, which may be the same or different. Non-limiting examples of an alkynyl group include —C═CH, —C═CH₃ and —C═CCH₂CH₃. The “R” portion of the alkynyl moiety may be branched, straight chain, or cyclic.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl. Any amine or carboxyl side chain on the compounds described herein can be amidified. The procedures and specific groups to make sure such amides are known to those skilled in the art and can readily be found in reference sources such as Greene and Wuts, Protective Groups in Organic Synthesis, 3 sup.rd. Ed., John Wiley & Sons, New York, N.Y., 1999, which is incorporated herein by reference in its entirety.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl group, or a bicyclic fused ring system. Bicyclic fused ring systems are exemplified by a phenyl group appended to the parent molecular moiety and fused to a cycloalkyl group, as defined herein, a phenyl group, a heteroaryl group, as defined herein, or a heterocycle, as defined herein. Representative examples of aryl include, but are not limited to, indolyl, naphthyl, phenyl, and tetrahydroquinolinyl.

The term “bond” or “single bond,” as used herein refers to a chemical bond between two atoms, or two moieties when the atoms joined by the bond are considered to be part of a larger substructure.

The term “cyano,” as used herein, refers to a —CN group.

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkyl,” as used herein, refers to a carbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl and cyclodecyl. “Cycloalkyl” also includes carbocyclic ring systems in which a cycloalkyl group is appended to the parent molecular moiety and is fused to an aryl group as defined herein (e.g., a phenyl group), a heteroaryl group as defined herein, or a heterocycle as defined herein. Representative examples of such cycloalkyl groups include, but are not limited to, 2,3-dihydro-1H-indenyl (e.g., 2,3-dihydro-1H-inden-1-yl and 2,3-dihydro-1H-inden-2-yl), 6,7-dihydro-5H-cyclopenta[b]pyridinyl (e.g., 6,7-dihydro-5H-cyclopenta[b]pyridin-6-yl), and 5,6,7,8-tetrahydroquinolinyl (e.g., 5,6,7,8-tetrahydroquinolin-5-yl). Illustrative examples of cycloalkyl groups include the following moieties:

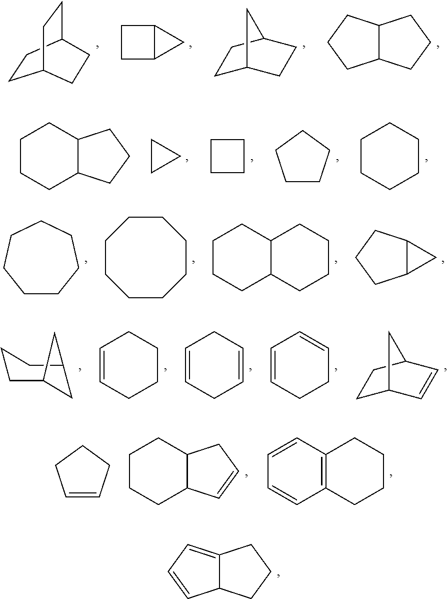

and the like.

The term “cycloalkenyl,” as used herein, means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl.

The term “ester” refers to a chemical moiety with formula COOR, where R is selected from the group consisting of alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic ring or an aromatic bicyclic ring system. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Representative examples of heteroaryl include, but are not limited to, indolyl, pyridinyl (including pyridin-2-yl, pyridin-3-yl, pyridin-4-yl), pyrimidinyl, pyrazinyl, pyridazinyl, pyrazolyl, pyrrolyl, benzopyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, benzimidazolyl, benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl, quinazolinyl, 1,2,4-triazinyl, 1,3,5-triazinyl, isoquinolinyl, quinolinyl, 6,7-dihydro-1,3-benzothiazolyl, imidazo[1,2-a]pyridinyl, naphthyridinyl, pyridoimidazolyl, thiazolo[5,4-b]pyridin-2-yl, thiazolo[5,4-d]pyrimidin-2-yl. Illustrative examples of heteroaryl groups include the following moieties:

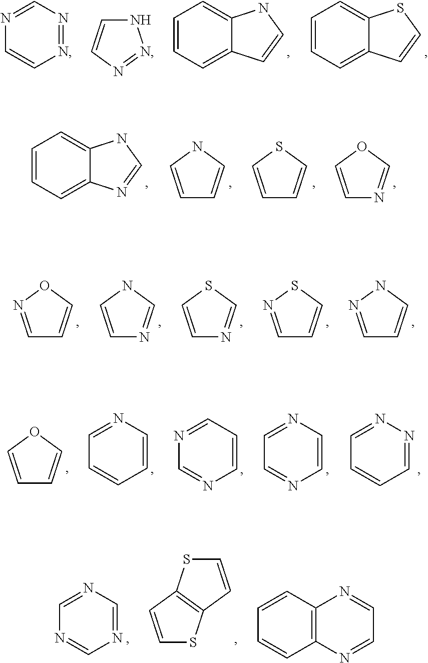

and the like.

The term “heterocycle” or “heterocyclic,” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocycles are connected to the parent molecular moiety through any carbon atom or any nitrogen atom contained within the rings, and can be unsubstituted or substituted. Illustrative examples of heterocycloalkyl groups, also referred to as heterocycle or heterocycloalkyl groups, include:

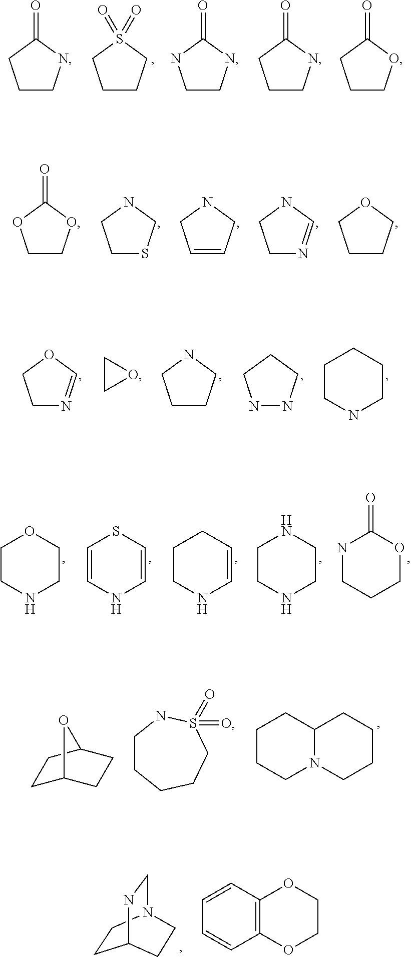

and the like. The term heterocycle also includes all ring forms of the carbohydrates, including but not limited to the monosaccharides, the disaccharides and the oligosaccharides.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

In some instances, the number of carbon atoms in a hydrocarbyl substituent (e.g., alkyl or cycloalkyl) is indicated by the prefix “Cₓ-C_{y}-”, wherein x is the minimum and y is the maximum number of carbon atoms in the substituent. Thus, for example, “C₁-C₃-alkyl” refers to an alkyl substituent containing from 1 to 3 carbon atoms.

The term “membered ring,” as used herein, can embrace any cyclic structure.

The term “isocyanato,” as used herein, refers to a —NCO group.

The term “isothiocyanato,” as used herein, refers to a —NCS group.

The term “mercaptyl,” as used herein, refers to a (alkyl)S— group.

The term “moiety,” as used herein refers to a specific segment or functional group of a molecule. Chemical moieties are often recognized chemical entities embedded in or appended to a molecule.

The term “sulfinyl,” as used herein, means —S(═O)—R, where R is selected from the group consisting of alkyl, cycloalkyl, aryl, heteroaryl (bonded through a ring carbon) and heteroalicyclic (bonded through a ring carbon).

The term “sulfonamide,” as used herein, means —S(O)₂NR^{d}— or —NR^{d}S(O)—, wherein R^{d} may be hydrogen, alkyl, cycloalkyl, aryl, heteroaryl, heterocycle, alkenyl, or heteroalkyl.

The term “sulfonyl,” as used herein, means —S(═O)₂—R, where R is selected from the group consisting of alkyl, cycloalkyl, aryl, heteroaryl (bonded through a ring carbon) and heteroalicyclic (bonded through a ring carbon).

The term “substituents” refers to a group “substituted” on an aryl, heteroaryl, phenyl or pyridinyl group at any atom of that group. Any atom can be substituted.

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl. For example, if a group is described as being “optionally substituted” (such as an alkyl, alkenyl, alkynyl, aryl, heteroaryl, cycloalkyl, heteroalkyl, heterocycle or other group such as an R group), it may have 0, 1, 2, 3, 4 or 5 substituents independently selected from halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

The term “thiocyanato,” as used herein, refers to a —CNS group.

The term “ [symbol] ” designates a single bond (—) or a double bond (═).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “acceptable” with respect to a formulation, composition or ingredient, as used herein, means having no persistent detrimental effect on the general health of the subject being treated.

The term “allosteric site” as used herein refers to a ligand binding site that is topographically distinct from the orthosteric binding site.

The term “ligand” as used herein refers to a natural or synthetic molecular entity that is capable of associating or binding to a receptor to form a complex and mediate, prevent or modify a biological effect. Thus, the term “ligand” encompasses allosteric modulators, inhibitors, activators, agonists, antagonists, natural substrates and analogs of natural substrates.

The terms “natural ligand” and “endogenous ligand” as used herein are used interchangeably, and refer to a naturally occurring ligand, found in nature, which binds to a receptor.

The term “agonist,” as used herein, refers to a molecule such as a compound, a drug, an enzyme activator or a hormone modulator which enhances the activity of another molecule or the activity of a receptor site.

The term “antagonist,” as used herein, refers to a molecule such as a compound, a drug, an enzyme inhibitor, or a hormone modulator, which diminishes, or prevents the action of another molecule or the activity of a receptor site.

The term “inverse agonist,” as used herein, refers to a molecule such as a compound, a drug, an enzyme inhibitor, or a hormone modulator, which diminishes, or prevents the basal level of activity (also sometimes referred to as constitutive activity) of another molecule or the activity of a receptor site.

The term “Selective Androgen Receptor Degrader”, or “SARDs” as used herein, refers to a molecule that interacts with the target, that is Androgen Receptor, either directly or indirectly, and results in the degradation of the receptor. The interactions include, but are not limited to, the interactions of an agonist and an antagonist.

The term “cancer,” as used herein, refers to an abnormal growth of cells which tend to proliferate in an uncontrolled way and, in some cases, to metastasize (spread). The types of cancer include, but is not limited to, solid tumors (such as those of the bladder, bowel, brain, breast, endometrium, heart, kidney, lung, lymphatic tissue (lymphoma), ovary, pancreas or other endocrine organ (thyroid), prostate, skin (melanoma) or hematological tumors (such as the leukemias).

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “enzymatically cleavable linker,” as used herein refers to unstable or degradable linkages which may be degraded by one or more enzymes.

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description. All references cited herein, including patents, patent applications, and publications, are hereby incorporated by reference in their entirety.

### 2. COMPOUNDS

In one aspect, disclosed is a compound of formula (I), or a pharmaceutically acceptable salt thereof,

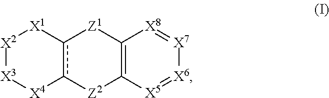

wherein X¹ is C(R^{1a}R^{1b}), O, S, C(O), S(O), or S(O)₂, or N—R^{1c}; X² is a bond, C(R^{2a}R^{2b}), or N—R^{2c}; X³ is a bond, C(R^{3a}R^{3b}), or N—R^{3c}; X⁴ is a bond, C(R^{4a}R^{4b}), C(R^{4a}R^{4b})—C(R^{4a}R^{4b}), C(O), S(O), S(O)₂, or N—R^{4c}; provided that no more than two of X²-X⁴ are simultaneously a bond; R^{1a}, R^{1b}, R^{2a}, R^{2b}, R^{3a}, R^{3b}, R^{4a}, and R^{4b} are each independently selected from the group consisting of hydrogen, deuterium, halogen, alkyl, alkenyl, alkynyl, heteroalkyl, aryl, heteroaryl, cycloalkyl, heterocycloalkyl, and —SO₂-alkyl; R^{1c}, R^{2c}, R^{3c}, and R^{4c} are each independently selected from the group consisting of hydrogen, deuterium, and alkyl; optionally R^{1a} and R^{1b}, R^{2a} and R^{2b}, R^{3a} and R^{3b}, R^{4a} and R^{4b}, R^{1a} and R^{2a}, R^{2a} and R^{3a}, R^{3a} and R^{4a}, R^{1c} and R^{2a}, R^{1c} and R^{2c}, R^{1a} and R^{2c}, R^{2c} and R^{3a}, R^{2c} and R^{3c}, R^{2a} and R^{3c}, R^{3c} and R^{4a}, R^{3c} and R^{4c}, or R^{3a} and R^{4c} together with the carbon atoms to which they are attached form an aryl, heteroaryl, cycloalkyl, cycloalkenyl, or heterocycloalkyl; X⁵ is C(R⁵), N, or a bond; X⁶ is C(R⁶) or N; X⁷ is C(R⁷) or N; X⁸ is C(R⁸) or N; Z¹ is C(R⁹R^{9′}); Z² is N—R¹⁰, C(R^{10′}R^{10″}), O, or S; R⁵, R⁶, R⁷, R⁸, R⁹, R^{10′}, and R^{10″} are each independently selected from the group consisting of hydrogen, deuterium, halogen, alkyl, alkenyl, alkynyl, heteroalkyl, aryl, heteroaryl, cycloalkyl, heterocycloalkyl, cyano, nitro, hydroxy, alkoxy, amino, alkylamino, and dialkylamino; R^{9′} is hydrogen or deuterium; optionally R⁵ and R⁶, R⁶ and R⁷, or R⁷ and R⁸ together with the carbon atoms to which they are attached form an aryl, heteroaryl, cycloalkyl, cycloalkenyl, or heterocycloalkyl; R¹⁰ is selected from the group consisting of hydrogen, deuterium, and alkyl; wherein said alkyl, alkenyl, alkynyl, heteroalkyl, alkoxy, aryl, heteroaryl, cycloalkyl, cycloalkenyl, and heterocycloalkyl, at each occurrence, whether alone or part of another group, are independently substituted with 0, 1, 2, 3, 4, or 5 substituents independently selected from the group consisting of deuterium, halogen, oxo (═O), ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycloalkyl, cycloalkylalkyl, cycloalkenylalkyl, heteroarylalkyl, arylalkyl, heterocycloalkylalkyl, hydroxy, hydroxyalkyl, alkoxy, alkylthio, alkoxyalkyl, alkylene, aryloxy, arylthio, phenoxy, benzyloxy, amino, alkylamino, dialkylamino, acylamino, aminoalkyl, arylamino, diarylamino, sulfonylamino, sulfinylamino, sulfonyl, alkylsulfonyl, arylsulfonyl, aminosulfonyl, sulfinyl, —COOH, ketone, alkoxycarbonyl, aryloxycarbonyl, amide, carbamate, acyl, boronic acid, and boronic ester.

In certain embodiments, the compounds of formula (I) have formula (I′):

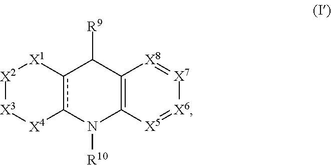

or a pharmaceutically acceptable salt thereof, wherein X¹ is C(R^{1a}R^{1b}), C(O), C(S), S(O), or S(O)₂; X² is a bond or C(R^{2a}R^{2b}); X³ is C(R^{3a}R^{3b}); X⁴ is C(R^{4a}R^{4b}), C(O), C(S), S(O), or S(O)₂; R^{1a}, R^{1b}, R^{2a}, R^{2b}, R^{3a}, R^{3b}, R^{4a}, and R^{4b} are each independently selected from the group consisting of hydrogen, halogen, alkyl, alkenyl, alkynyl, aryl, heteroaryl, cycloalkyl, heterocycloalkyl, and —SO₂-alkyl; X⁵ is C(R⁵) or N; X⁶ is C(R⁶) or N; X⁷ is C(R⁷) or N; X⁸ is C(R⁸) or N; R⁵, R⁶, R⁷, and R⁸ are each independently selected from the group consisting of hydrogen, halogen, alkyl, alkenyl, alkynyl, aryl, heteroaryl, cycloalkyl, heterocycloalkyl, cyano, nitro, hydroxy, alkoxy, amino, alkylamino, and dialkylamino; optionally R⁵ and R⁶, R⁶ and R⁷, or R⁷ and R⁸ together with the carbon atoms to which they are attached form a 5- or 6-membered aryl, heteroaryl, cycloalkyl, cycloalkenyl, or heterocycloalkyl; R⁹ is aryl, heteroaryl, cycloalkyl, or heterocycloalkyl; and R¹⁰ is hydrogen or alkyl; wherein said alkyl, alkenyl, alkynyl, alkoxy, aryl, heteroaryl, cycloalkyl, and heterocycloalkyl, at each occurrence, whether alone or part of another group, are independently substituted with 0, 1, 2, 3, 4, or 5 substituents independently selected from the group consisting of halogen, oxo (═O), ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycloalkyl, cycloalkylalkyl, cycloalkenylalkyl, heteroarylalkyl, arylalkyl, heterocycloalkylalkyl, hydroxy, hydroxyalkyl, alkoxy, alkylthio, alkoxyalkyl, alkylene, aryloxy, arylthio, phenoxy, benzyloxy, amino, alkylamino, dialkylamino, acylamino, aminoalkyl, arylamino, diarylamino, sulfonylamino, sulfinylamino, sulfonyl, alkylsulfonyl, arylsulfonyl, aminosulfonyl, sulfinyl, —COOH, ketone, alkoxycarbonyl, aryloxycarbonyl, amide, carbamate, acyl, boronic acid, and boronic ester.

In certain embodiments, X¹ is C(O). In certain embodiments, X¹ is C(S). In certain embodiments, X¹ is S(O)₂.

In certain embodiments, X² is C(R^{2a}R^{2b}); R^{2a} is hydrogen; and R^{2b} is hydrogen.

In certain embodiments, X³ is C(R^{3a}R^{3b}); R^{3a} is hydrogen, C₁-C₄-alkyl, or C₁-C₄-haloalkyl; and R^{3b} is hydrogen, C₁-C₄-alkyl, or C₁-C₄-haloalkyl.

In certain embodiments, X³ is C(R^{3a}R^{3b}); R^{3a} is methyl; and R^{3b} is methyl.

In certain embodiments, X³ is N—R^{3c}. In certain embodiments, X³ is N—CH₃.

In certain embodiments, X⁴ is C(R^{4a}R^{4b}); R^{4a} is hydrogen; and R^{4b} is hydrogen.

In certain embodiments, X⁵ is C(R⁵); X⁶ is C(R⁶); X⁷ is C(R⁷); and X⁸ is C(R⁸).

In certain embodiments, X⁶ is C(R⁶), and R⁶ is

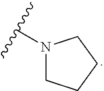 [SMILES: *N1CCCC1]

In certain embodiments, R⁷ and R⁸, together with the carbon atoms to which they are attached form a 6-membered aryl, heteroaryl, cycloalkyl, cycloalkenyl, or heterocycloalkyl.

In certain embodiments, R⁷ and R⁸, together with the carbon atoms to which they are attached form a 6-membered aryl or heteroaryl containing one or two nitrogen atoms.

In certain embodiments, R⁷ and R⁸, together with the carbon atoms to which they are attached form a 6-membered aryl or heteroaryl containing one nitrogen atom.

In certain embodiments, R⁷ and R⁸, together with the carbon atoms to which they are attached form a 5-membered heteroaryl, cycloalkyl, cycloalkenyl, or heterocycloalkyl.

In certain embodiments, X⁵ is C(R⁵); X⁶ is C(R⁶); X⁷ is C(R⁷); X⁸ is C(R⁸); and one of R⁵ and R⁶, or R⁷ and R⁸, together with the carbon atoms to which they are attached form a 5- or 6-membered aryl or heteroaryl.

In certain embodiments, X⁵ is CH; X⁶ is CH; X⁷ is C(R⁷); X⁸ is C(R⁸); and R⁷ and R⁸, together with the carbon atoms to which they are attached form a 5- or 6-membered aryl or heteroaryl containing one nitrogen atom.

In certain embodiments, X⁵ is CH; X⁶ is CH; X⁷ is C(R⁷); X⁸ is C(R⁸); and R⁷ and R⁸, together with the carbon atoms to which they are attached form a 5- or 6-membered cycloalkyl, cycloalkenyl, or heterocycloalkyl.

In certain embodiments, X⁷ is C(R⁷); X⁸ is C(R⁸); and R⁷ and R⁸, together with the carbon atoms to which they are attached form a 5-membered group selected from:

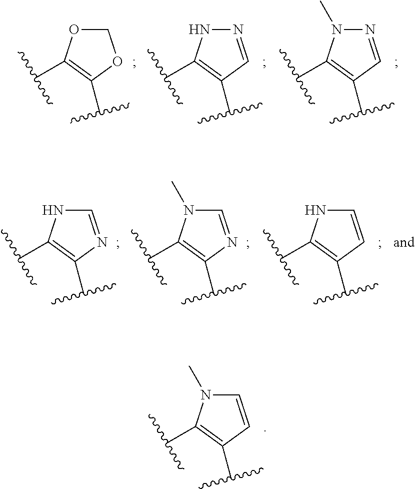

In certain embodiments, X⁷ is C(R⁷); X⁸ is C(R⁸); and R⁷ and R⁸, together with the carbon atoms to which they are attached form a 6-membered group selected from:

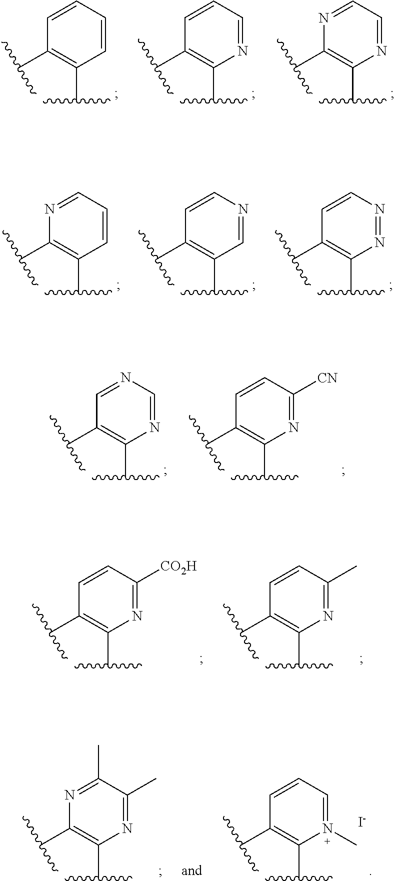

In certain embodiments, X⁶ is C(R⁶); X⁷ is C(R⁷); and R⁶ and R⁷, together with the carbon atoms to which they are attached form a 6-membered cycloalkyl.

In certain embodiments, R⁹ is monocyclic aryl, bicyclic aryl, monocyclic heteroaryl, or bicyclic heteroaryl, wherein aryl and heteroaryl are substituted with 0, 1, 2, 3, 4, or 5 substituents independently selected from the group consisting of halogen, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycloalkyl, cycloalkylalkyl, cycloalkenylalkyl, heteroarylalkyl, arylalkyl, heterocycloalkylalkyl, hydroxy, hydroxyalkyl, alkoxy, alkylthio, alkoxyalkyl, alkylene, aryloxy, arylthio, phenoxy, benzyloxy, amino, alkylamino, dialkylamino, acylamino, aminoalkyl, arylamino, diarylamino, sulfonylamino, sulfinylamino, sulfonyl, alkylsulfonyl, arylsulfonyl, aminosulfonyl, sulfinyl, —COOH, ketone, alkoxycarbonyl, aryloxycarbonyl, amide, carbamate, acyl, boronic acid, and boronic ester.

In certain embodiments, R⁹ is

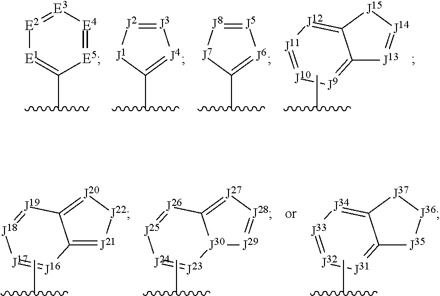

wherein E¹-E⁵ are each independently CR²⁰ or N, wherein R²⁰, at each occurrence, is independently selected from the group consisting of hydrogen, halogen, hydroxy, cyano, amino, nitro, C₁-C₆-alkyl, C₂-C₆-alkenyl, C₂-C₆-alkynyl, C₁-C₆-alkoxy, C₁-C₆-haloalkyl, C₁-C₆-haloalkoxy, C₁-C₆-alkylamino, C₁-C₆-dialkylamino, C₁-C₆-heteroalkyl, C₁-C₆-alkylsulfonyl, —COR²¹, and —B(OR²²)₂; wherein R²¹ is selected from the group consisting of hydrogen, hydroxy, C₁-C₆-alkyl, C₁-C₆-alkoxy, C₁-C₆-haloalkyl, and C₁-C₆-haloalkoxy; wherein R²², at each occurrence, is independently selected from the group consisting of hydrogen, C₁-C₆-alkyl, and C₁-C₆-haloalkyl; J¹ and J⁵ are O, S, or NR²³, wherein R²³ is hydrogen, C₁-C₆-alkyl, or C₁-C₆-haloalkyl; J¹⁵ and J²² are O, S, NR²⁴, or C(═O), wherein R²⁴ is hydrogen, C₁-C₆-alkyl, or C₁-C₆-haloalkyl; J³⁰ is N; J²-J⁴, J⁶-J¹⁴, J¹⁶-J²¹, J²³-J²⁹, and J³¹-J³⁴ are each independently CR²⁵ or N, wherein R²⁵, at each occurrence, is independently selected from the group consisting of hydrogen, halogen, hydroxy, cyano, amino, nitro, C₁-C₆-alkyl, C₂-C₆-alkenyl, C₂-C₆-alkynyl, C₁-C₆-alkoxy, C₁-C₆-haloalkyl, C₁-C₆-haloalkoxy, C₁-C₆-alkylamino, C₁-C₆-dialkylamino, and C₁-C₆-heteroalkyl; J³⁵-J³⁷ are each independently CR²⁶R²⁷, NR²⁸, O, and C(═O), wherein R²⁶ and R²⁷, at each occurrence, are independently selected from the group consisting of hydrogen, halogen, hydroxy, cyano, amino, nitro, C₁-C₆-alkyl, C₂-C₆-alkenyl, C₂-C₆-alkynyl, C₁-C₆-alkoxy, C₁-C₆-haloalkyl, C₁-C₆-haloalkoxy, C₁-C₆-alkylamino, C₁-C₆-dialkylamino, and C₁-C₆-heteroalkyl, and wherein R²⁸ is hydrogen, C₁-C₆-alkyl, or C₁-C₆-haloalkyl; provided that J³⁶ is not O when one of J³⁵ or J³⁷ is O; provided that J³⁶ is not C(═O) when one of J³⁵ or J³⁷ is C(═O); provided that one of J⁹-J¹⁴ is C where the R⁹ attaches to the parent molecular formula; provided that one of J¹⁶-J²¹ is C where the R⁹ attaches to the parent molecular formula; provided that one of J²³-J²⁹ is C where the R⁹ attaches to the parent molecular formula; and provided that one of J³¹-J³⁴ is C where the R⁹ attaches to the parent molecular formula.

In certain embodiments, R⁹ is selected from the group consisting of:

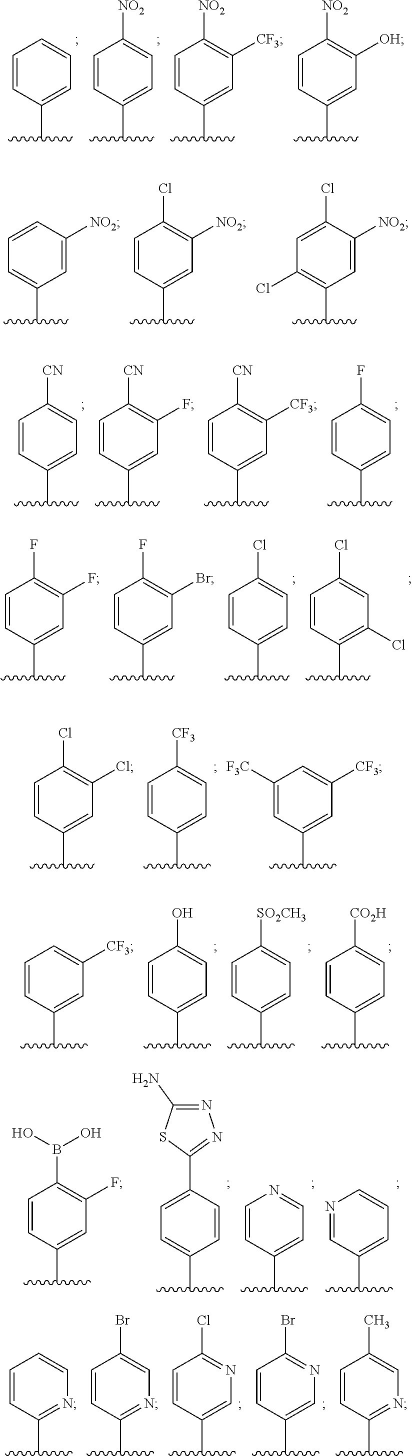

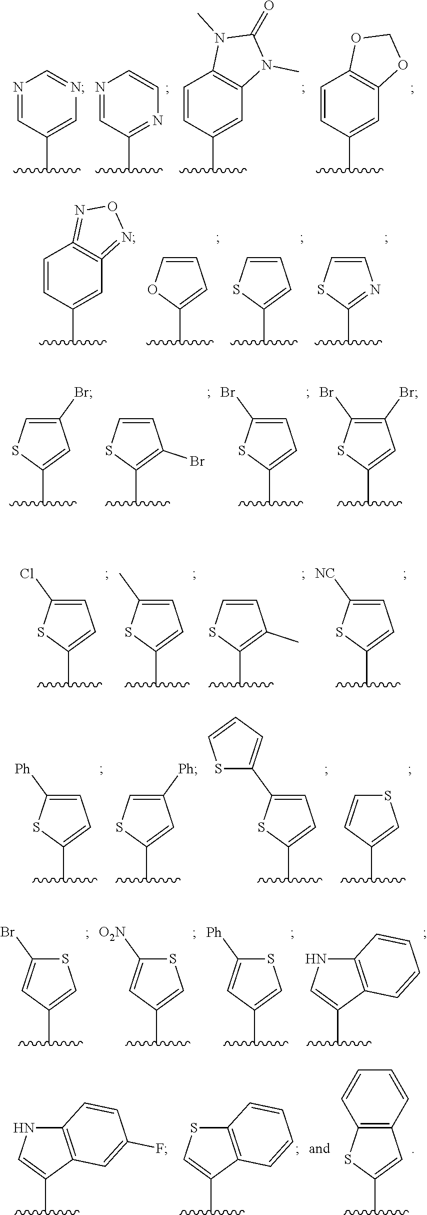

In certain embodiments, R^{9′} is hydrogen. In certain embodiments, R^{9′} is deuterium.

In certain embodiments, R¹⁰ is hydrogen.

In certain embodiments, the compound of formula (I) or (I′) has formula:

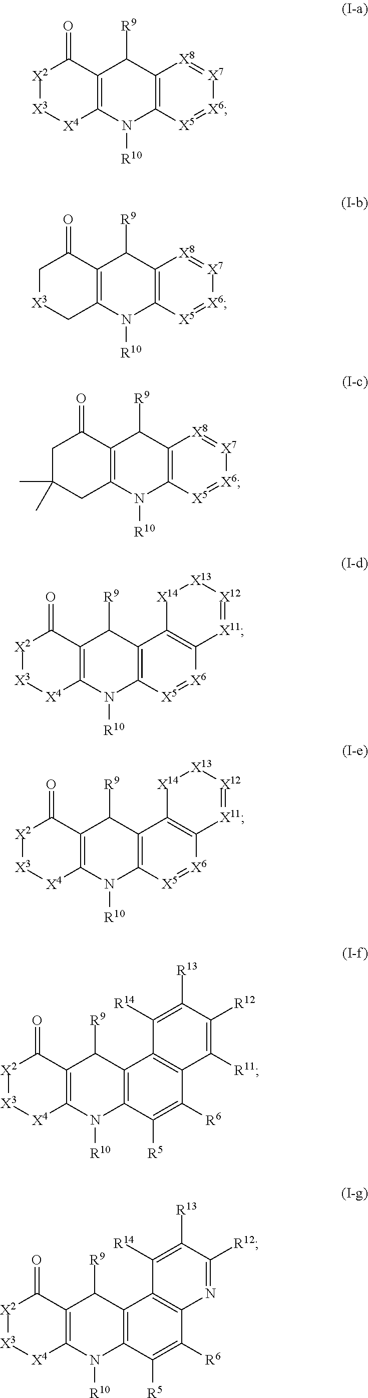

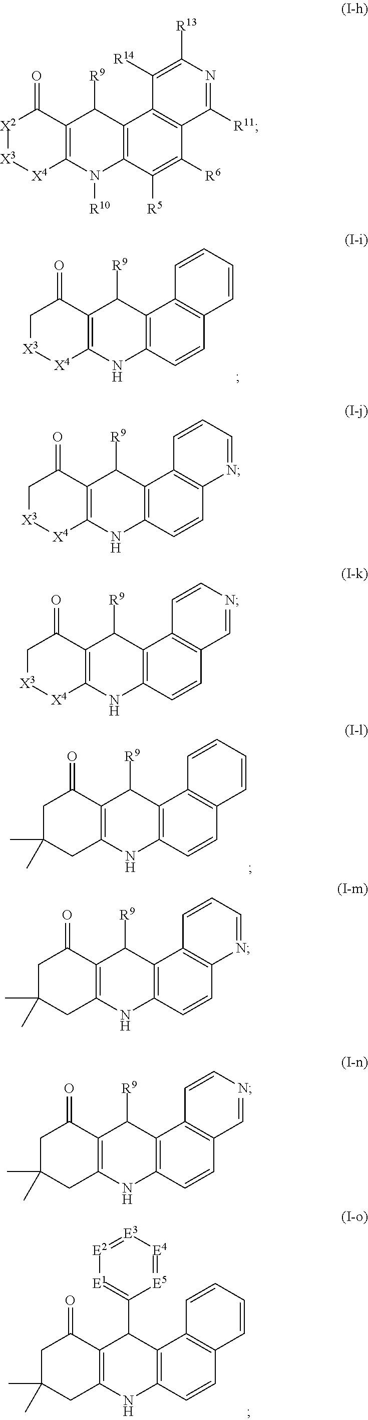

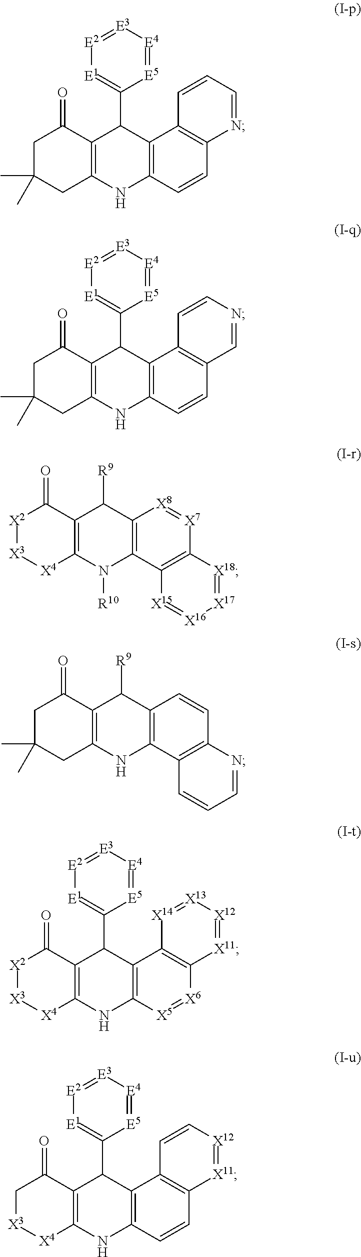

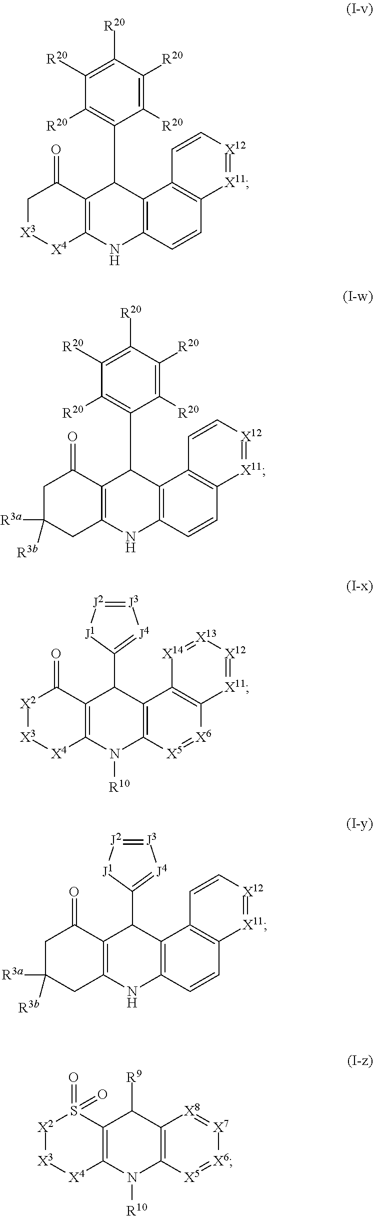

wherein X¹¹ is C(R¹¹) or N; X¹² is C(R¹²) or N; X¹³ is C(R¹³) or N; X¹⁴ is C(R¹⁴) or N; X¹⁵ is C(R¹⁵) or N; X¹⁶ is C(R¹⁶) or N; X¹⁷ is C(R¹⁷) or N; X¹⁸ is C(R¹⁸) or N; R¹¹, R¹², R¹³, R¹⁴, R¹⁵, R¹⁶, R¹⁷, and R¹⁸ are each independently selected from the group consisting of hydrogen, halogen, alkyl, alkenyl, alkynyl, aryl, heteroaryl, cycloalkyl, and heterocycloalkyl; E¹-E⁵ are each independently CR²⁰ or N, wherein R²⁰, at each occurrence, is independently selected from the group consisting of hydrogen, halogen, hydroxy, cyano, amino, nitro, C₁-C₆-alkyl, C₂-C₆-alkenyl, C₂-C₆-alkynyl, C₁-C₆-alkoxy, C₁-C₆-haloalkyl, C₁-C₆-haloalkoxy, C₁-C₆-alkylamino, C₁-C₆-dialkylamino, C₁-C₆-heteroalkyl, C₁-C₆-alkylsulfonyl, —COR²¹, and —B(OR²²)₂; wherein R²¹ is selected from the group consisting of hydrogen, hydroxy, C₁-C₆-alkyl, C₁-C₆-alkoxy, C₁-C₆-haloalkyl, and C₁-C₆-haloalkoxy; wherein R²², at each occurrence, is independently selected from the group consisting of hydrogen, C₁-C₆-alkyl, and C₁-C₆-haloalkyl; J¹ is O, S, or NR²³, wherein R²³ is hydrogen, C₁-C₆-alkyl, or C₁-C₆-haloalkyl; and J²-J⁴ are each independently CR²⁵ or N, wherein R²⁵, at each occurrence, is independently selected from the group consisting of hydrogen, halogen, hydroxy, cyano, amino, nitro, C₁-C₆-alkyl, C₂-C₆-alkenyl, C₂-C₆-alkynyl, C₁-C₆-alkoxy, C₁-C₆-haloalkyl, C₁-C₆-haloalkoxy, C₁-C₆-alkylamino, C₁-C₆-dialkylamino, and C₁-C₆-heteroalkyl.

In certain embodiments, a compound of the present disclosure is selected from the group consisting of a compound as defined in Table 1, or a pharmaceutically acceptable salt thereof.

In certain embodiments, disclosed is a compound selected from the group consisting of:

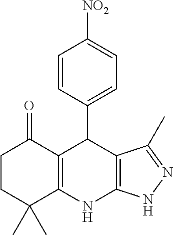 [SMILES: Cc1n[nH]c2c1C(c1ccc([N+](=O)[O-])cc1)C1=C(N2)C(C)(C)CCC1=O]

3,8,8-trimethyl-4-(4-nitrophenyl)-1,4,6,7,8,9-hexahydro-5H-pyrazolo[3,4-b]quinolin-5-one; or a pharmaceutically acceptable salt thereof. This compound is representative of when X⁵ is a bond in formula (I).

In another aspect, disclosed is the compound of formula (II):

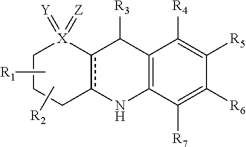

wherein X is selected from the group consisting of C and S, Y is either O or nothing, Z is O, R₁ and R₂ are selected from the group consisting of alkyl and aryl, R₃ is

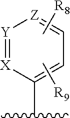

wherein X, Y and Z are C or N, R₈ and R₉ are selected from the groups consisting of alkyl, halogens, fused alkyl and fused aryl, R₄ and R₅ are selected from group consisting of linear or fused alkyl, aryl and heteroaryl; R₆ and R₇ are selected from group consisting of linear or fused alkyl, aryl and heteroaryl.

In certain embodiments, the compound of formula (II) has formula:

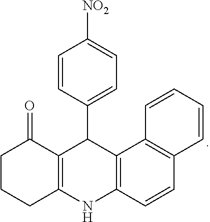 [SMILES: O=C1CCCC2=C1C(c1ccc([N+](=O)[O-])cc1)c1c(ccc3ccccc13)N2]

In certain embodiments, the compound of formula (II) has formula:

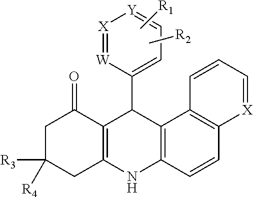

wherein W, X, Y and Z are selected from the group consisting of C and N.

In certain embodiments, the compound of formula (II) has formula:

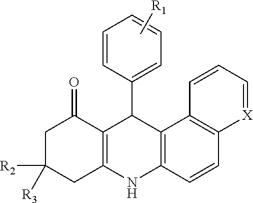

wherein X is selected from the group consisting of C and N.

In certain embodiments, the compound of formula (II) has formula:

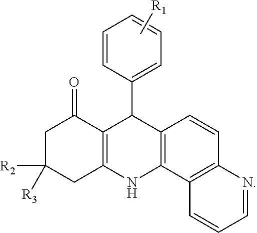

In certain embodiments, the compound of formula (II) has formula:

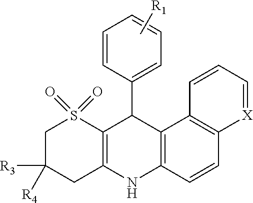

wherein X is selected from the group consisting of C and N.

In certain embodiments, the compound of formula (II) has formula:

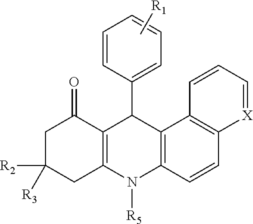

wherein X is selected from the group consisting of C and N.

In certain embodiments, the fused aryl comprises the following structure:

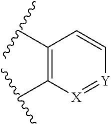

wherein X and Y are each C or N.

In another aspect, disclosed are compounds of formula:

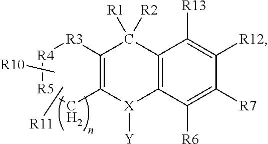

wherein X is selected from the group consisting of N, C, S, O, and alkyl, Y is either hydrogen, deuterium, O, or nothing, n represented in the structure as

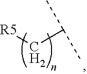

is a number from zero(0) to two(2), R1 is selected from the group consisting of hydrogen, deuterium, alkyl, fused alkyl, heteroalkyl, aryl, fused aryl, heteroaryl, and halogen, R2 is hydrogen or deuterium, R3 is selected from

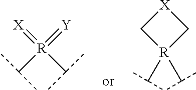

wherein R represents R3 and is selected from the group consisting of C, N, and S, X is selected from the group consisting of O, and S, and Y is either O, or nothing, R4 and R5 are independently selected from the group consisting of C, and N, R10 and R11 are independently selected from a group consisting of hydrogen, deuterium, alkyl, and aryl, R6 and R7 are independently selected from a group consisting of hydrogen, alkyl, fused alkyl, heteroalkyl, aryl, fused aryl, heteroaryl, and halogen, R12 and R13 are independently selected from a group consisting of linear or fused alkyl, heteroalkyl, aryl, and heteroaryl.

In another aspect, disclosed are compounds of formula:

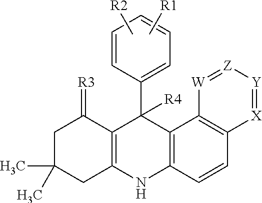

wherein R1, and R2 are independently selected from the group consisting of hydrogen, deuterium, alkyl, fused alkyl, heteroalkyl, aryl, fused aryl, heteroaryl, and halogen, R3 is selected from the group consisting of S, and O, R4 is selected from group consisting of hydrogen, and deuterium, and W, X, Y, and Z are independently selected from the groups consisting of C and N.

In another aspect, disclosed are compounds of formula:

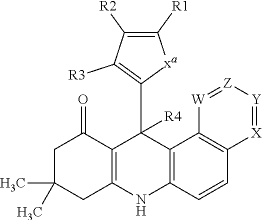

wherein R1-R3 are independently selected from the group consisting of hydrogen, deuterium, alkyl, fused alkyl, heteroalkyl, aryl, fused aryl, heteroaryl, and halogen, R4 is hydrogen or deuterium, W, X, Y, and Z are independently selected from the group consisting of C and N, and X^{a} is O or S.

In another aspect, disclosed are compounds of formula:

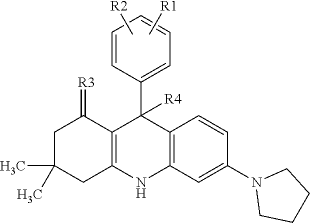

wherein R1, and R2 are independently selected from the group consisting of hydrogen, deuterium, alkyl, fused alkyl, heteroalkyl, aryl, fused aryl, heteroaryl, and halogen, and R4 is hydrogen or deuterium.

In another aspect, disclosed are compounds of formula:

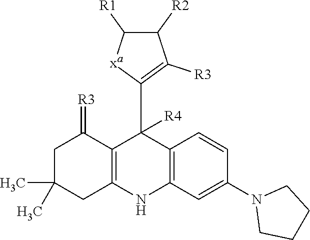

wherein R1-R3 are independently selected from the group consisting of hydrogen, deuterium, alkyl, fused alkyl, heteroalkyl, aryl, fused aryl, heteroaryl, and halogen, R4 is hydrogen or deuterium, and X^{a} is O or S.

In another aspect, disclosed are compounds of formula

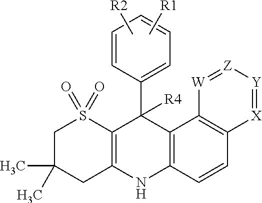

wherein R1, and R2 are independently selected from the group consisting of hydrogen, deuterium, alkyl, fused alkyl, heteroalkyl, aryl, fused aryl, heteroaryl, and halogen, R4 is hydrogen or deuterium, and W, X, Y, and Z are independently selected from the group consisting of C and N.

In another aspect, disclosed are compounds of formula

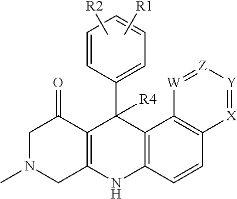

wherein R1, and R2 are independently selected from the group consisting of hydrogen, deuterium, alkyl, fused alkyl, heteroalkyl, aryl, fused aryl, heteroaryl, and halogen, R4 is hydrogen or deuterium, and W, X, Y, and Z are independently selected from the group consisting of C and N.

In another aspect, disclosed are compounds of formula

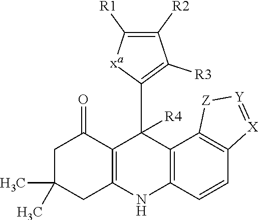

wherein R1-R3 are independently selected from the group consisting of hydrogen, deuterium, alkyl, fused alkyl, heteroalkyl, aryl, fused aryl, heteroaryl, halogen, nitrile, and nitro, R4 is hydrogen or deuterium, X, Y, and Z are independently selected from the group consisting of C and N, and X^{a} is S or O.

In another aspect, disclosed are compounds of formula

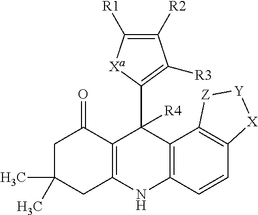

wherein R1-R3 are independently selected from the group consisting of hydrogen, deuterium, alkyl, fused alkyl, heteroalkyl, aryl, fused aryl, heteroaryl, and halogen, R4 is hydrogen or deuterium, X, Y, and Z are independently selected from the group consisting of C and N, and X^{a} is O or S.

In certain embodiments, one or more of the following compounds are excluded:
12-(4-nitrophenyl)-8,9,10,12-tetrahydrobenzo[a]acridin-11(7H)-one (Compound A1);
9,9-dimethyl-12-(4-nitrophenyl)-8,9,10,12-tetrahydrobenzo[b][4,7]phenanthrolin-11(7H)-one (Compound A2);
9,9-dimethyl-12-(4-nitrophenyl)-8,9,10,12-tetrahydrobenzo[a]acridin-11(7H)-one (Compound A51);
9,9-dimethyl-12-(pyridin-2-yl)-8,9,10,12-tetrahydrobenzo[b][4,7]phenanthrolin-11(7H)-one (Compound A57);
9,9-dimethyl-12-(thiophen-2-yl)-8,9,10,12-tetrahydrobenzo[b][4,7]phenanthrolin-11(7H)-one (Compound A66); or
9,9-dimethyl-12-(5-methylthiophen-2-yl)-8,9,10,12-tetrahydrobenzo[a]acridin-11(7H)-one (Compound A106).

Compound names are assigned by using Struct=Name naming algorithm as part of CHEMDRAW® ULTRA v. 12.0.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the invention.

The present disclosure also includes an isotopically-labeled compound, which is identical to those recited in formula (I) or formula (II), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### 3. SYNTHETIC METHODS

The disclosed compounds may be prepared by synthetic processes or by metabolic processes. Preparation of the compounds by metabolic processes includes those occurring in the human or animal body (in vivo) or processes occurring in vitro.

A representative pathway for the synthesis of compounds described herein comprises the following:

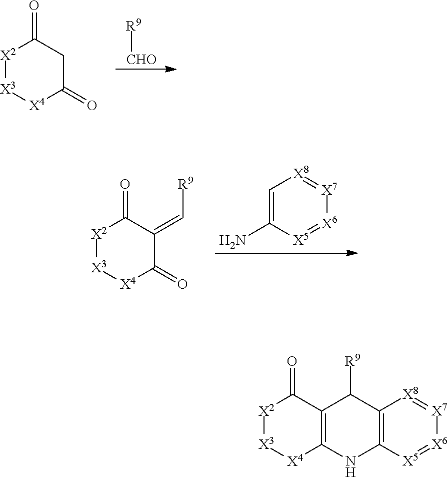

For example, one exemplary method comprises the following:

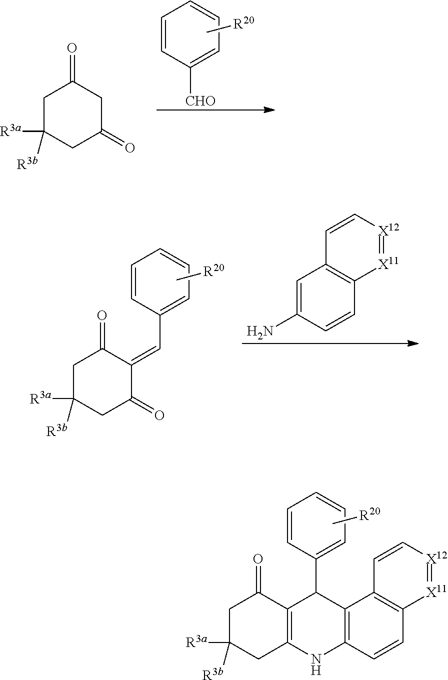

Another exemplary method comprises the following:

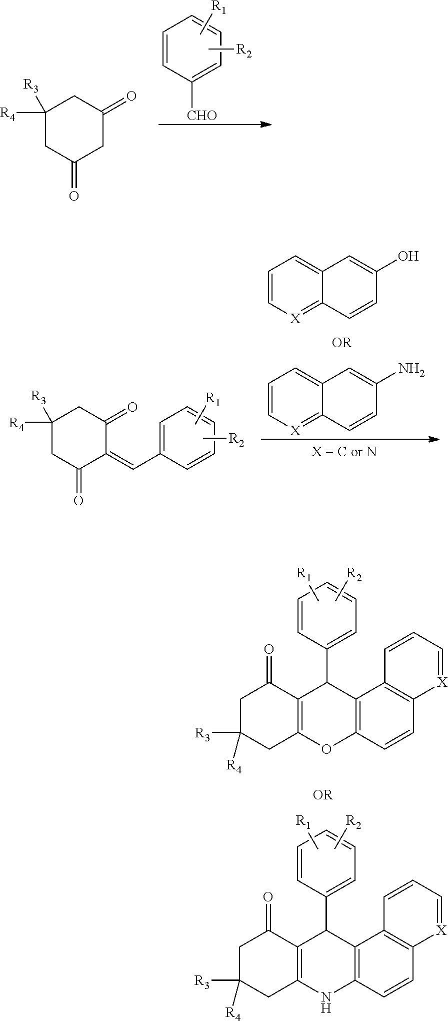

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 4. PHARMACEUTICAL COMPOSITIONS

The disclosed compounds may be incorporated into pharmaceutical compositions suitable for administration to a subject (such as a patient, which may be a human or non-human).

The said compounds according to the present disclosure can be used in their final non-salt form. On the other hand, the present disclosure also encompasses the use of these compounds in the form of their pharmaceutically acceptable salts, which can be derived from various organic and inorganic acids and bases by procedures known in the art. By “pharmaceutically acceptable,” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained. Pharmaceutically acceptable salts may be obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts may also be obtained by reacting a compound described herein, with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

With regard to that stated above, it can be seen that the expression “pharmaceutically acceptable salt” in the present connection is taken to mean an active compound which comprises a compound described herein in the form of one of its salts, in particular if this salt form imparts improved pharmacokinetic properties on the active compound compared with the free form of the active compound or any other salt form of the active compound used earlier. The pharmaceutically acceptable salt form of the active compound can also provide this active compound for the first time with a desired pharmacokinetic property which it did not have earlier and can even have a positive influence on the pharmacodynamics of this active compound with respect to its therapeutic efficacy in the body.

The present disclosure furthermore relates to the use of the compounds and/or physiologically acceptable salts thereof for the preparation of a medicament (pharmaceutical composition), in particular by non-chemical methods. They can be converted into a suitable dosage form here together with at least one solid, liquid and/or semi-liquid excipient or adjuvant and, if desired, in combination with one or more further active compounds. The term “pharmaceutical composition” refers to a mixture of a compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The present disclosure furthermore relates to medicaments comprising at least one compound described herein and/or pharmaceutically usable derivatives, solvates and stereoisomers thereof, including mixtures thereof in all ratios, and optionally excipients and/or adjuvants. The term “pharmaceutical combination” or “pharmaceutical formulation” are used interchangeably herein and refer to a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

Pharmaceutical formulations can be administered in the form of dosage units which comprise a predetermined amount of active compound per dosage unit. In certain embodiments, the unit may be, for example, about 0.1 mg to about 3 g, preferably about 1 mg to about 700 mg, particularly preferably about 5 mg to about 100 mg, of a compound according to the present disclosure, depending on the condition treated, the method of administration and the age, weight and condition of the patient, or pharmaceutical formulations can be administered in the form of dosage units which comprise a predetermined amount of active compound per dosage unit. Preferred dosage unit formulations are those which comprise a daily dose or part-dose, as indicated above, or a corresponding fraction thereof of an active compound. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active compound with the excipient(s) or adjuvant(s). Techniques and formulations may generally be found in “Remington's Pharmaceutical Sciences”, (Meade Publishing Co., Easton, Pa.). Therapeutic compositions must typically be sterile and stable under the conditions of manufacture and storage.

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavor, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrate or solubilize, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape, which are broken up to form granules. The granules can be lubricated by addition of stearic acid, a stearate salt, talc or mineral oil in order to prevent sticking to the tablet casting molds. The lubricated mixture is then pressed to give tablets. The compounds according to the invention can also be combined with a free-flowing ineli excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compound. Syrups can be prepared by dissolving the compound in an aqueous solution with a suitable flavor, while elixirs are prepared using a non-toxic alcoholic vehicle. Suspensions can be formulated by dispersion of the compound in a non-toxic vehicle. Solubilizers and emulsifiers, such as, for example, ethoxylated isostearyl alcohols and polyoxyethylene sorbitol ethers, preservatives, flavor additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds provided herein and their salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds provided herein and their salts, solvates and physiologically functional derivatives thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxyethylaspatartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active compound can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active compound can be employed either with a paraffinic or a water-miscible cream base. Alternatively, the active compound can be formulated to give a cream with an oil-in-water cream base or a water-in-oil base.

Pharmaceutical formulations adapted for topical application to the eye include eye drops, in which the active compound is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurized dispensers with aerosols, nebulizers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilized) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavors.

A therapeutically effective amount of a compound provided herein depends on a number of factors, including, for example, the age and weight of the human or animal, the precise condition that requires treatment, and its severity, the nature of the formulation and the method of administration, and is ultimately determined by the treating doctor or vet. However, an effective amount of a compound according to the invention for the treatment is generally in the range from about 0.1 to about 100 mg/kg of body weight of the recipient (mammal) per day and particularly typically in the range from about 1 to about 10 mg/kg of body weight per day. Thus, the actual amount per day for an adult mammal weighing about 70 kg is usually between about 70 and about 700 mg, where this amount can be administered as a single dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se. It can be assumed that similar doses are suitable for the treatment of the other conditions mentioned above.

In certain embodiments, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The present disclosure further relates to medicaments comprising at least one compound provided herein and/or pharmaceutically usable derivatives, solvates and stereoisomers thereof, including mixtures thereof in all ratios, and optionally excipients and/or adjuvants and at least one further medicament active compound.

### 5. METHODS OF TREATMENT

The disclosed compounds and compositions may be used in methods for treatment of cancer, tumor growth, metastatic growth and the like The methods of treatment may comprise administering to a subject in need of such treatment a composition comprising a therapeutically effective amount of the compound of formula (I) or formula (II).

In one aspect, disclosed is a method of treating cancer, the method comprising administration of a therapeutically effective amount of a compound of formula (I) or formula (II), or a pharmaceutically acceptable salt thereof to a subject in need thereof.

In certain embodiments, the cancer being treated is associated with dysfunction of androgen receptors.

In certain embodiments, the cancer is at least one of melanoma (e.g., unresectable, metastatic melanoma), renal cancer (e.g., renal cell carcinoma), prostate cancer (e.g., metastatic castration resistant prostate cancer, hormone refractory prostate cancer), ovarian cancer (e.g., epithelial ovarian cancer, such as metastatic epithelial ovarian cancer), breast cancer (e.g., triple negative breast cancer), glioblastoma, and lung cancer (e.g., non-small cell lung cancer), soft tissue sarcoma, fibrosarcoma, osteosarcoma, pancreatic cancer, colorectal cancer, pancreatic cancer, gastric cancer, stomach cancer, cancers of the blood and cancers of the lymphatic systems, among others.

In certain embodiments, the disease is a solid tumor.

In certain embodiments, the solid tumor is selected from the group consisting of where the tumor is selected from the group consisting of tumors of the squamous epithelium, the bladder, the stomach, the kidneys, the head and neck, the esophagus, the cervix, the thyroid, of the intestine, the liver, the brain, the prostate, the urogenital tract, the lymphatic system, the larynx and/or the lung, lung adenocarcinoma, small-cell lung carcinoma, pancreatic cancer, glioblastoma, colon carcinoma, breast carcinoma, tumor of the blood and immune system, acute myeloid leukemia, chronic myeloid leukemia, acute lymphatic leukemia, chronic lymphatic. In certain embodiments, the tumor is in the prostate.

In certain embodiments, the compositions may be useful for the treatment of solid tumors, where a therapeutically effective amount of a compound such as an androgen receptor modulator and/or further angiogenesis inhibitors.

The compositions may be useful for treating certain cancers in humans and animals related to androgen receptor dysfunction. Treatment of such cancers can be effected by modulating androgen receptors in a subject, by administering a compound or composition of the invention, either alone or in combination with another active agent as part of a therapeutic regimen to a subject in need thereof.

In certain embodiments, the compositions may be suitable for combination with known anticancer agents. These known anticancer agents include the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors and further angiogenesis inhibitors.

In certain embodiments, a therapeutically effective amount of a compound of the formula (I) is administered in combination with radiotherapy, and/or androgen receptor modulator, and/or cytotoxic agents, and/or further angiogenesis inhibitors. In certain embodiments, the present compounds are particularly suitable for administration at the same time as radiotherapy.

In certain embodiments, the compositions are preferably chemotherapeutic agents, in particular those which inhibit angiogenesis and thus inhibit growth and spread of tumor cells; preference is given here to VEGF receptor inhibitors, including robozymes and antisense which are directed to VEGF receptors, and angiostatin and endostatin.

Additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I). The above combinations include combinations of a compound of the present invention not only with one other active compound, but also with two or more other active compounds. For example, the compound of Formula (I) can be combined with a variety of different anti-cancer drugs such as chemotherapeutics, anti-tumor agents, and anti-proliferative agents.

In certain embodiments, the compositions are administered in combination with an antineoplastic agent. Examples of antineoplastic agents which can be used in combination with the compositions generally include alkylating agents, antimetabolites; epidophyllotoxin; an antineoplastic enzyme; a topoisomerase inhibitor; procarbazin; mitoxantron or platinum coordination complexes. In certain embodiments, antineoplastic agents are preferably selected from the following classes: anthracyclins, vinca medicaments, mitomycins, bleomycins, cytotoxic nucleosides, epothilones, discodermolides, pteridines, diynenes and podophyllotoxins.

Preference is given in the said classes to, for example, caminomycin, daunornbicin, aminopterin, methotrexate, methopterin, dichloromethotrexate; mitomycin C, porfiromycin, 5-fluorouracil, 5-fluorodeoxyuridine monophosphate, cytarabines, 5-azacytidine, thioguanine, azathioprines, adenosine, pentostatin, erythrohydroxynonyladenine, cladribines, 6-mercaptopurine, gemcitabine, cytosinarabinoside, podophyllotoxin or podophyllotoxin derivatives, such as, for example, etoposide, etoposide phosphate or teniposide, melphalan, vinblastine, vincristine, leurosidine, vindesine, leurosine and paclitaxel. Other preferred antineoplastic agents are selected from the group estramustine, carboplatin, cyclophosphamide, bleomycin, gemcitabine, ifosamide, melphalan, hexamethylmelamine, thiotepa, cytarabin, idatrexate, trimetrexate, dacarbazine, L-asparaginase, camptothecin, CPT-11, topotecan, arabinosylcytosine, bicalutamide, flutamide, leuprolide, pyridobenzoindole derivatives, interferons and interleukins.

In certain embodiments, the compositions may be administered in combination with antibiotics, including but not limited to dactinomycin, daunorubicin, idarubicin, epirubicin, mitoxantrone, bleomycin, plicamycin, mitomycin and combinations thereof.

In certain embodiments, the compositions may be administered in combination with enzyme inhibitors. Examples of suitable enzyme inhibitors include, but are not limited to, histone deacetylation inhibitors (for example suberoyl anilide hydroxamic acid [SAHA]) and the tyrosine kinase inhibitors (for example ZD 1839 [Iressa]).

In certain embodiments, the compositions may be administered in combination with nuclear export inhibitors. Nuclear export inhibitors prevent the expression of biopolymers (for example RNA) from the cell nucleus. Examples include those selected from the group callystatin, leptomycin B, ratjadone.

In certain embodiments, the compositions may be administered in combination with immunosuppressants, such as those selected from the group rapamycin, CCl-779 (Wyeth), RADOOl (Novartis), AP23573 (Ariad Pharmaceuticals).

The disclosed compounds and compositions may be used in methods for treatment of prostate cancer, benign prostatic hyperplasia, hypersexuality, male contraception, acne, amenorrhea, seborrhea, hirsutism, androgenic alopecia, hidradenitis suppurativa, and hyperandrogenism, and for trans women undergoing sex reassignment.

The disclosed compounds may be included in kits comprising separate packs of (a) an effective amount of a compound provided herein and/or pharmaceutically usable derivatives, solvates and stereoisomers thereof, including mixtures thereof in all ratios, and (b) an effective amount of a further medicament active compound.

The kit may further comprise suitable containers, such as boxes, individual bottles, bags or ampoules. The set may, for example, comprise separate ampoules, each containing an effective amount of a compound provided herein and/or pharmaceutically usable derivatives, solvates and stereoisomers thereof, including mixtures thereof in all ratios, and an effective amount of a further medicament active compound in dissolved or lyophilized form.

The term “androgen receptor modulators,” as used herein, may refer to compounds which interfere with or inhibit the binding of androgens to the receptor, regardless of mechanism. Examples of androgen receptor modulators include finasteride and other 5α-reductase inhibitors, nilutamide, flutamide, bicalutamide, liarozole and abiraterone acetate. Androgen receptor modulators may also refer to selective androgen receptor modulators and selective androgen receptor degraders.

The term “cytotoxic agents,” as used herein, refers to compounds which result in cell death primarily through direct action on the cellular function or inhibit or interfere with cell myosis, including alkylating agents, tumor necrosis factors, intercalators, microtubulin inhibitors and topoisomerase inhibitors.

Examples of cytotoxic agents include, but are not limited to, tirapazimine, sertenef, cachectin, ifosfamide, tasonelmin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPXl 00, (trans, trans, trans)bis-mu-(hexane-1,6-diamine)-mu [diamineplatinum(II)]bis [diamine(chloro)platinum(II)]tetrachloride, diarisidinylspermine, arsenic trioxide, 1-(11-dodecylamino-1 O-hydroxyundecyl)-3, 7-dimethy lxanthine, zorubicin, idarubicin, daunorubicin, bisantrene, mitoxantrone, pirarubicin, pinafide, vahubicin, amrubicin, antineoplaston, 3′-deamino-3′-morpholino-13-deoxo-1 0-hydroxycaminomycin, annamycin, galarubicin, and elinafide.

Examples of microtubulin inhibitors include paclitaxel, vindesine sulfate, 3′,′4′-didehydro-4′-deoxy-8′-norvincaleuko blastine, docetaxol, rhizoxin, dolastatin, mivobulin, isethionate, auristatin, cemadotin, RPR109881, BMS184476, vinflunine, cryptophycin, 2,3,4,5,6-pentafluoro-N-(3-fluoro-4-methoxyphenyl) benzenesulfonamide, anhydrovinblastine, N,N-dimethyl-L-valyl-L-valyl-N-methyl-L-valyl-L-prolyl-L-proline-t-butylamide, TDX258 and BMS188797.

Topoisomerase inhibitors are, for example, topotecan, hycaptamine, irinotecan, rubitecan, 6-ethoxypropionyl-3′,4′-0-exobenzylidenechartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H-benzo[de-]pyrano[3′,4′:b,7]indolizino[1,2b]quinoline-10,13(9H, 15H)-dione, lurtotecan, 7-[2-(N-isopropylamino)ethyl]-(20S)camptothecin, BNP 1350, BNPil 100, BN80915, BN80942, etoposide phosphate, teniposide, sobuzoxane, 2′-dimethylamino-2′-deoxyetoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[-4 hydroxy-3,5-dimethoxyphenyl]-5,5a,6,8,8a,9-hexohydrofuro(3′,4′:6, 7)naphtho(2,3-d)-1,3-dioxol-6-one, 2,3-(methylenedioxy)-5-methyl-7-hydroxy-8-methoxybenzo[c]phenanthridinium-,6,9-bis[(2-aminoethyl)amino]benzo [g]isoquinoline-5,10-dione, 5-(3-aminopropylamino)-7,10-dihydroxy-2-(2-hydroxyethylaminomethyl)-6H-pyrazolo[4,5, 1-de]-acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c] quinolin-7-one and dimesna.

“Antiproliferative agents” include antisense RNA and DNA oligonucleotides such as G3139, ODN698, RVASKRAS, GEM231 and INX3001 and anti-metabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′ deoxycytidine, N-[5-(2,3-dihydro benzofuryl)sulfonyl]-N′-(3,4-dichlorophenyl)urea, N6-[4-deoxy-4-[N2-[2(E),4(E)-tetradecadienoyl]glycylamino]-L-glycero-B-L-mannoheptopyranosyl]adenine, aplidine, ecteinascidin, troxacitabine, 4-[2-amino-4-oxo-4,6, 7,8-tetrahydro-3H-pyrimidino[5,4-b]-1,4-thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)tetradeca-2,4, 6-trien-9-ylacetic acid ester, swainsonine, lometrexol, dexrazoxane, methioninase, 2′-cyano-2′-deoxy-N4-palmitoyl-1-B-D-arabinofuranosyl cytosine and 3-aminopyridine-2-carboxaldehyde thiosemicarbazone. “Antiproliferative agents” also include monoclonal antibodies to growth factors other than those listed under “angiogenesis inhibitors”, such as trastuzumab, and tumor suppressor genes, such as p53, which can be delivered, for example, via recombinant virus-mediated gene transfer.

### 6. EXAMPLES

The compounds and processes of the invention will be better understood by reference to the following examples, which are intended as an illustration of and not a limitation upon the scope of the invention.

### Example 1

### (±)-12-(3,4-difluorophenyl)-9,9-bis(trideuteromethyl)-8,9,10,12-tetrahydrobenzo[b][4,7]phenanthrolin-11(7H)-one (Compound A49)

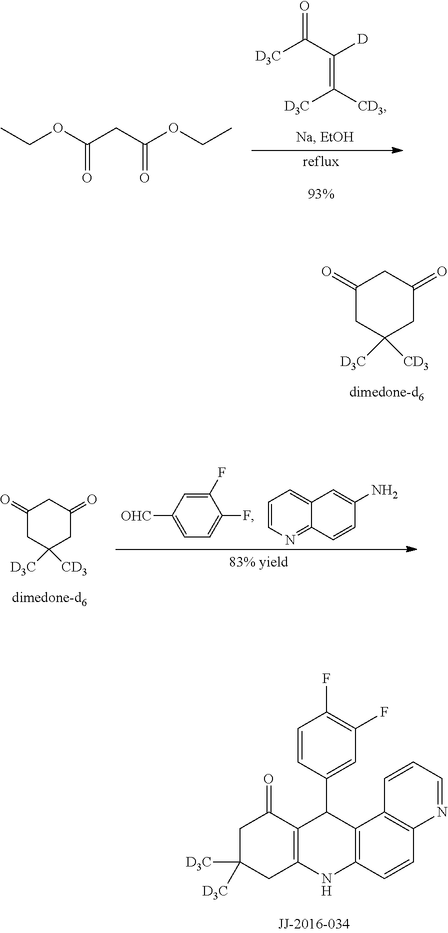

3 mL of anhydrous ethanol was added to a 50-mL flask charged with sodium (120 mg, 5.2 mmol) at RT. After being stirred for 30 min, dimethylmalonate (0.8 mL, 5.5 mmol) was added. The resulting mixture was stirred for 10 min, and then mesityl-d₁₀ oxide (0.5 mL, 4.6 mmol) was added. The reaction mixture was then heated 2 hours at reflux. After being cooled to RT, an aqueous solution of KOH (626 mg, 11.2 mmol, in 3 mL water) was added. The resulting mixture was re-heated for 3 hours at reflux. The reaction was then cooled to RT, acidified with 6N HCl aqueous solution to pH=1, extracted with EtOAc (100 mL). The organic layer was washed with brine (20 mL), dried over Na₂SO₄, concentrated, and purified on silica gel flash chromatography (DCM:MeOH=15:1), giving the dimedone-d₆ (630 mg, 93% yield). Then JJ-2016-034 was synthesized in 83% yield from dimedone-d₆ following the representative procedure.

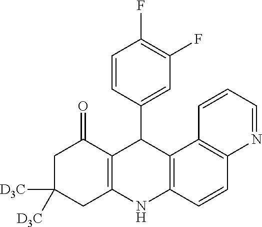 [SMILES: [2H]C([2H])([2H])C1(C([2H])([2H])[2H])CC(=O)C2=C(C1)Nc1ccc3ncccc3c1C2c1ccc(F)c(F)c1]

¹H NMR (400 MHz, DMSO-d₆) δ 9.91 (s, 1H), 8.68 (dd, J=4.0 and 1.2 Hz, 1H), 8.38 (d, J=8.8 Hz, 1H), 7.91 (d, J=8.8 Hz, 1H), 7.56 (d, J=9.2 Hz, 1H), 7.39 (dd, J=8.8 and 4.4 Hz, 1H), 7.30 (ddd, J=11.6, 8.0 and 2.0 Hz, 1H), 7.17 (dt, J=10.8 and 8.4 Hz, 1H), 6.97 (ddd, J=8.4, 4.0 and 2.0 Hz, 1H), 5.84 (s, 1H), 2.55 (d, J=16.8 Hz, 1H), 2.31 (d, J=16.8 Hz, 1H), 2.23 (d, J=16.0 Hz, 1H), 2.04 (d, J=16.0 Hz, 1H). ¹³C NMR (100 MHz, DMSO-d₆) δ 193.4 (s, 1C), 150.9 (s, 1C), 148.8 (dd, J=244 and 13 Hz, 1C), 147.7 (s, 1C), 147.5 (dd, J=243 and 13 Hz, 1C), 145.5 (s, 1C), 144.4 (t, J=4 Hz, 1C), 134.5 (s, 1C), 130.7 (s, 1C), 129.4 (s, 1C), 126.4 (s, 1C), 124.0 (dd, J=6 and 3 Hz, 1C), 121.9 (s, 1C), 120.5 (s, 1C), 117.0 (d, J=17 Hz, 1C), 116.3 (d, J=17 Hz, 1C), 115.5 (s, 1C), 106.4 (s, 1C), 50.1 (s, 1C), 35.0 (s, 2C), 31.7 (m, 1C). ¹⁹F NMR (376 MHz, DMSO-d₆) δ −139.4 (m, 1F), −142.4 (m, 1F).

### Example 2

### (±)-12-(3,4-difluorophenyl)-9,9-dimethyl-8,9,10,12-tetrahydro-7H-thiopyrano[2,3-b][4,7]phenanthroline 11,11-dioxide (Compound A53)

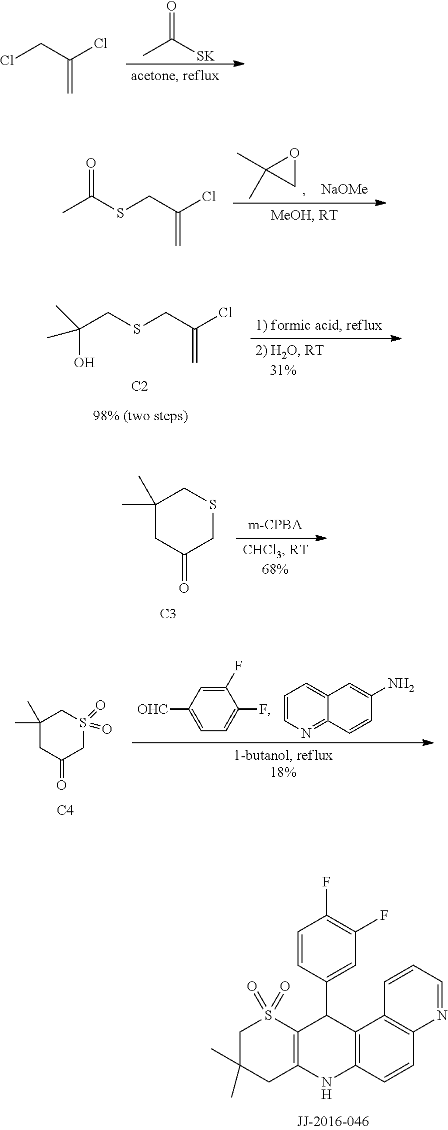

To a suspension of potassium thioacetate (1.2 g, 10.8 mmol) in acetone (10 mL) was added 2,3-dichloropropnene (1.0 g, 9.0 mmol). The resulting mixture was heated for 3 hours at reflux, then cooled to RT, diluted with water, extracted with DCM. The organic layer was dried over Na₂SO₄, filtered through a silica pad, concentrated, to afford the C1, which was used in the next step without further purification.

The above C1 was dissolved in 10 mL absolute methanol. To this solution was added sodium methanolate (464 mg, 8.6 mmol). The reaction mixture was stirred for 40 min at RT, and then was added 2,2-dimethyl-oxirane (618 mg, 8.6 mmol). The reaction mixture was stirred for 16 hours at RT before being quenched by water. The quenched reaction mixture was adjusted to pH=7 with 1N HCl aqueous solution and extracted with EtOAc (100 mL). The organic layer was washed with brine, dried over Na₂SO₄, concentrated, and purified on silica gel flash chromatography (Hexane:DCM:EtOAc=5:5:1), giving the C2 (1.5 g, 98% yield for two steps).

C2 (1.4 g, 7.9 mmol) was reflux in formic acid (10 mL) for 4 hours. The reaction solution was cooled RT and poured in cold water (10 mL). The mixture was extracted with EtOAc (100 mL), washed with water, brine, dried over Na₂SO₄, concentrated, and purified on silica gel flash chromatography (Hexane:DCM:EtOAc=5:5:1), giving the C3 (360 mg, 31% yield).

m-CPBA (316 mg, 77%, 1.8 mmol) was added to the solution of C3 in CHCl₃ (10 mL) at RT. After being stirred for 3 hours at RT, additional m-CPBA (220 mg, 77%, 0.9 mmol) was added. The reaction mixture was further stirred for 1 hour, then diluted with DCM. The mixture was washed with 1M NaHCO₃ aqueous solution (80 mL), The organic layer was washed with water, brine, dried over Na₂SO₄, concentrated, and purified on silica gel flash chromatography (DCM:EtOAc=1:1), giving the C4 (179 mg, 68% yield).

Then JJ-2016-046 was synthesized in 18% yield from C4 following the representative procedure.

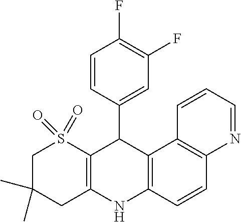 [SMILES: CC1(C)CC2=C(C(c3ccc(F)c(F)c3)c3c(ccc4ncccc34)N2)S(=O)(=O)C1]

¹H NMR (400 MHz, CD₃OD) δ 8.64 (dd, J=4.4 and 0.8 Hz, 1H), 8.48 (d, J=8.8 Hz, 1H), 7.89 (d, J=8.8 Hz, 1H), 7.48 (d, J=8.8 Hz, 1H), 7.46 (dd, J=8.8 and 4.4 Hz, 1H), 7.24 (ddd, J=11.2, 7.6 and 2.0 Hz, 1H), 7.14 (ddd, J=7.6, 3.2 and 2.0 Hz, 1H), 7.04 (dt, J=10.4 and 8.4 Hz, 1H), 5.95 (s, 1H), 3.23 (d, J=13.6 Hz, 1H), 3.12 (d, J=13.6 Hz, 1H), 2.58 (d, J=16.8 Hz, 1H), 2.51 (d, J=16.8 Hz, 1H), 1.24 (s, 3H), 1.22 (s, 3H). ¹³C NMR (100 MHz, CD₃OD) δ 151.3 (dd, J=245 and 13 Hz, 1C), 150.4 (dd, J=244 and 13 Hz, 1C), 148.4 (s, 1C), 146.5 (s, 1C), 144.9 (t, J=4 Hz, 1C), 142.3 (s, 1C), 136.4 (s, 1C), 132.7 (s, 1C), 129.9 (s, 1C), 127.9 (s, 1C), 125.2 (dd, J=6 and 3 Hz, 1C), 123.1 (s, 1C), 121.9 (s, 1C), 117.9 (d, J=18 Hz, 1C), 117.7 (d, J=17 Hz, 1C), 115.5 (s, 1C), 106.8 (s, 1C), 63.2 (s, 1C), 41.2 (s, 1C), 35.8 (s, 1C), 32.9 (s, 1C), 30.1 (s, 1C), 26.8 (s, 1C). ¹⁹F NMR (376 MHz, CD₃OD) δ −140.7 (m, 1F), −143.3 (m, 1F).

### Example 3

### (±)-12-(3,4-difluorophenyl)-9-methyl-8,9,10,12-tetrahydropyrido[4,3-b][4,7]phenanthrolin-11(7H)-one (Compound A54)

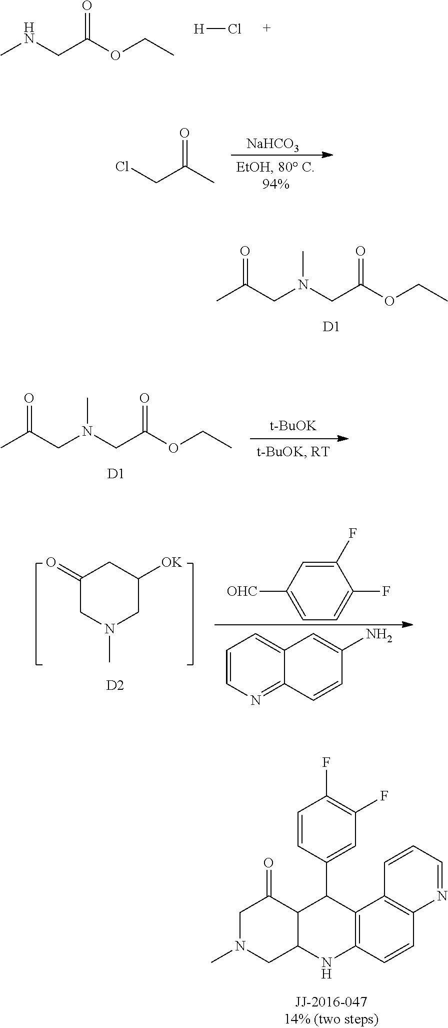

To a solution of sarcosine ethyl ester hydrochloride (3.1 g, 20 mmol) in ethanol (30 mL) was added NaHCO₃ (3.4 g, 40 mmol) and chloroacetone (1.6 mL, 20 mmol). The reaction mixture was stirred for 24 hours at reflux, then cooled to RT. The precipitate was filtered off, and the filtrate was diluted with EtOAc (125 mL), concentrated HCl (8 mL) and water (30 mL) were added. The mixture was stirred for 15 min, and the phases were separated. The aqueous phase was added NaHCO₃ until pH=10, the extracted with EtOAc (120 mL). The organic layer was washed with water, brine, dried over Na₂SO₄, concentrated, and purified on silica gel flash chromatography (DCM:MeOH=10:1), giving the D1 (3.2 g, 94% yield).

To a solution of t-BuOK (239 mg, 2.1 mmol) in THF (5 mL) was added a solution of D1 (351 mg, 2 mmol) in THF (3 mL) dropwise at 0° C. After the addition, the resulting mixture was stirred for 20 hours at RT and then concentrated to afford the crude D2, which was used in the next step without further purification.

Then JJ-2016-047 was synthesized in 14% yield from D2 following the representative procedure.

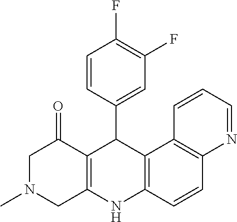 [SMILES: CN1CC(=O)C2=C(C1)Nc1ccc3ncccc3c1C2c1ccc(F)c(F)c1]

¹H NMR (400 MHz, CDCl₃) δ 8.78 (d, J=4.0 Hz, 1H), 8.21 (d, J=8.8 Hz, 1H), 8.00 (d, J=9.2 Hz, 1H), 7.44 (s, 1H), 7.35 (dd, J=8.0 and 4.0 Hz, 1H), 7.33 (d, J=8.8 Hz, 1H), 7.03 (m, 2H), 6.93 (q, J=8.8 Hz, 1H), 6.01 (s, 1H), 3.55 (d, J=15.2 Hz, 1H), 3.32 (t, J=16.0 Hz, 2H), 2.99 (d, J=16.0 Hz, 1H), 2.40 (s, 3H). ¹³C NMR (100 MHz, CDCl₃) δ 192.1 (s, 1C), 150.3 (dd, J=247 and 13 Hz, 1C), 149.8 (s, 1C), 148.9 (dd, J=246 and 13 Hz, 1C), 148.5 (s, 1C), 146.3 (s, 1C), 142.3 (t, J=4 Hz, 1C), 133.5 (s, 1C), 131.4 (s, 1C), 130.2 (s, 1C), 127.1 (s, 1C), 123.8 (dd, J=6 and 3 Hz, 1C), 122.1 (s, 1C), 119.8 (s, 1C), 116.9 (d, J=7 Hz, 1C), 116.8 (d, J=7 Hz, 1C), 116.5 (s, 1C), 108.0 (s, 1C), 62.8 (s, 1C), 54.7 (s, 1C), 44.6 (s, 1C), 34.9 (s, 1C). ¹⁹F NMR (376 MHz, CDCl₃) δ −137.6 (m, 1F), −141.2 (m, 1F).

### Example 4

### (±)-12-(3,4-difluorophenyl)-9,9-dimethyl-11-oxo-7,8,9,10,11,12-hexahydrobenzo[b][4,7]phenanthroline-3-carbonitrile (Compound A52)

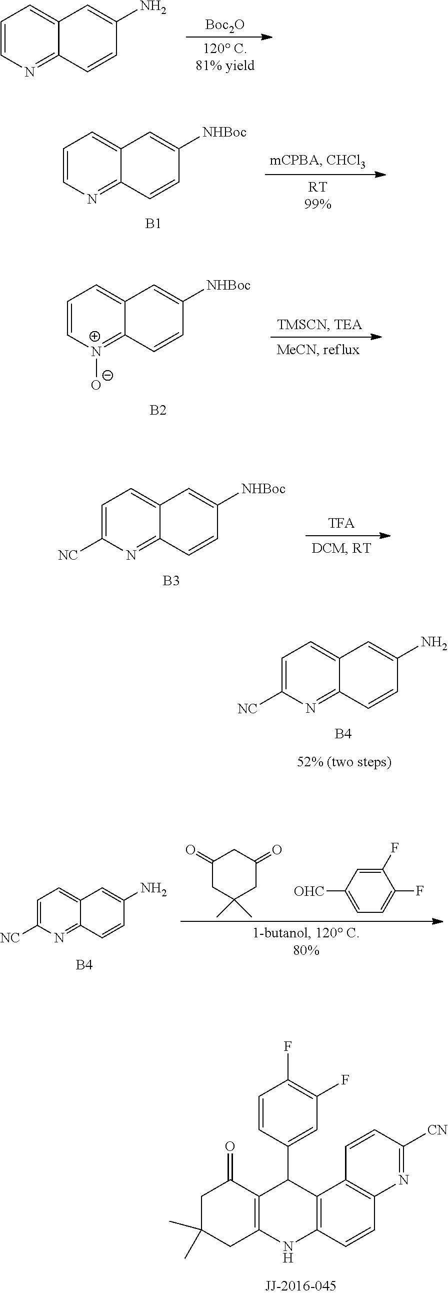

A mixture of 6-aminoquinoline (1.73 g, 12 mmol) and Boc₂O (3.91 g, 18 mmol) was stirred for 30 min at 120° C. To the reaction mixture, silica gel (12 mL) and toluene (40 mL) were added, followed by stirring for 1 hour at 80° C. The reaction mixture was cooled to RT, and purified by silica gel flash chromatography (hexane:EtOAc=1:1), giving the B1 (2.4 g, 81 yield).

To a solution of the B1 (488 mg, 2 mmol) in CHCl₃, m-CPBA (77% w/w, 448 mg, 2 mmol) was added in portions at RT. The resulting mixture was stirred for 1 hour at RT, then diluted with EtOAc (60 mL), washed with saturated NaHCO₃ aqueous solution and brine, dried over Na₂SO₄, concentrated, and purified on silica gel flash chromatography (DCM:MeOH=20:1), giving the B2 (520 mg, 99% yield).

To a solution of the B2 (390 mg, 1.5 mmol) in MeCN, Et₃N (630 μL, 4.5 mmol) and TMS-CN (300 μL, 2.3 mmol) were added at RT. The resulting mixture was stirred for 1 hour at reflux, concentrated in vacuum to afford the B3, which was used in the next step without further purification.

The above B3 was dissolved in 10 mL DCM. After being cooled to 0° C., 1.5 mL (20 mmol) of TFA was added slowly. The reaction mixture was stirred for 20 hours at RT, the concentrated. The residue was dissolved in EtOAc (60 mL), washed with saturated NaHCO₃ aqueous solution and brine, dried over Na₂SO₄, concentrated, and purified on silica gel flash chromatography (DCM:EtOAc=10:1), giving the B4 (131 mg, 52% yield for two steps). Then JJ-2016-045 was synthesized in 80% yield from B4 following the representative procedure.

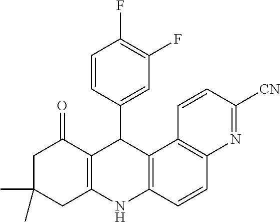 [SMILES: CC1(C)CC(=O)C2=C(C1)Nc1ccc3nc(C#N)ccc3c1C2c1ccc(F)c(F)c1]

¹H NMR (400 MHz, DMSO-d₆) δ 10.14 (s, 1H), 8.60 (d, J=8.4 Hz, 1H), 8.00 (d, J=9.2 Hz, 1H), 7.85 (d, J=8.8 Hz, 1H), 7.69 (d, J=9.2 Hz, 1H), 7.34 (ddd, J=11.6, 10.0 and 2.0 Hz, 1H), 7.19 (dt, J=10.4 and 8.8 Hz, 1H), 6.97 (m, 1H), 5.83 (s, 1H), 2.57 (d, J=16.8 Hz, 1H), 2.43 (d, J=16.8 Hz, 1H), 2.25 (d, J=16.0 Hz, 1H), 2.06 (d, J=16.0 Hz, 1H), 1.04 (s, 3H), 0.85 (s, 3H). ¹³C NMR (100 MHz, DMSO-d₆) δ 193.6 (s, 1C), 150.4 (s, 1C), 148.9 (dd, J=244 and 13 Hz, 1C), 147.6 (dd, J=243 and 12 Hz, 1C), 145.3 (s, 1C), 143.8 (t, J=4 Hz, 1C), 137.0 (s, 1C), 132.6 (s, 1C), 129.8 (s, 1C), 129.6 (s, 1C), 127.7 (s, 1C), 124.4 (s, 1C), 124.1 (dd, J=6 and 3 Hz, 1C), 122.8 (s, 1C), 117.8 (s, 1C), 117.1 (d, J=7 Hz, 1C), 116.5 (d, J=7 Hz, 1C), 115.1 (s, 1C), 107.0 (s, 1C), 50.1 (s, 1C), 34.9 (s, 1C), 32.2 (s, 2C), 29.0 (s, 1C), 26.2 (s, 1C). ¹⁹F NMR (376 MHz, DMSO-d₆) δ −139.2 (m, 1F), −142.1 (m, 1F).

### Example 5

### (±)-12-(3,4-difluorophenyl)-9,9-dimethyl-11-oxo-7,8,9,10,11,12-hexahydrobenzo[b][4,7]phenanthroline-3-carboxylic acid (Compound A80)

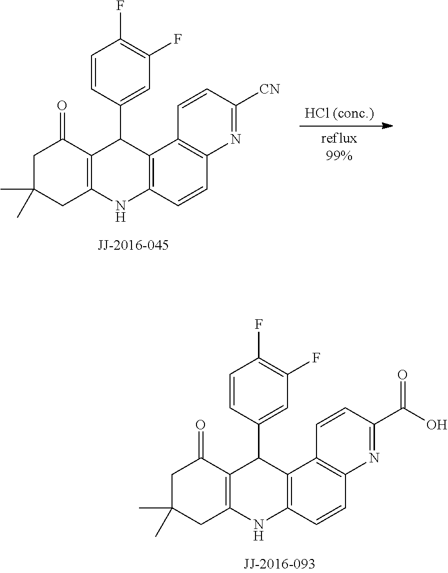 [SMILES: CC1(C)CC(=O)C2=C(C1)Nc1ccc3nc(C#N)ccc3c1C2c1ccc(F)c(F)c1 ; CC1(C)CC(=O)C2=C(C1)Nc1ccc3nc(C(=O)O)ccc3c1C2c1ccc(F)c(F)c1]

JJ-2016-045 (42 mg, 1 mmol) was reflux in concentrated HCl (3 mL) for 20 hours. The reaction mixture was cooled to RT, concentrated, recrystallized from a solution of EtOH/EtOAc, giving the JJ-2016-093 (43 mg, 99% yield).

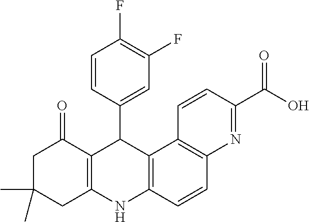 [SMILES: CC1(C)CC(=O)C2=C(C1)Nc1ccc3nc(C(=O)O)ccc3c1C2c1ccc(F)c(F)c1]

¹H NMR (400 MHz, DMSO-d₆) δ 10.04 (s, 1H), 8.54 (d, J=8.8 Hz, 1H), 8.00 (q, J=9.2 Hz, 1H), 7.64 (d, J=9.2 Hz, 1H), 7.31 (ddd, J=11.6, 8.0 and 2.0 Hz, 1H), 7.19 (dt, J=10.4 and 8.8 Hz, 1H), 6.97 (m, 1H), 5.85 (s, 1H), 2.57 (d, J=16.4 Hz, 1H), 2.43 (d, J=16.4 Hz, 1H), 2.25 (d, J=16.0 Hz, 1H), 2.07 (d, J=16.0 Hz, 1H), 1.04 (s, 3H), 0.86 (s, 3H). ¹³C NMR (100 MHz, DMSO-d₆) δ 193.5 (s, 1C), 166.2 (s, 1C), 150.7 (s, 1C), 148.9 (dd, J=244 and 13 Hz, 1C), 147.6 (dd, J=243 and 13 Hz, 1C), 145.7 (s, 1C), 144.6 (s, 1C), 144.1 (t, J=4 Hz, 1C), 136.2 (s, 1C), 132.0 (s, 1C), 130.3 (s, 1C), 127.7 (s, 1C), 124.0 (q, J=3 Hz, 1C), 121.7 (s, 1C), 121.4 (s, 1C), 117.0 (d, J=7 Hz, 1C), 116.3 (d, J=7 Hz, 1C), 115.1 (s, 1C), 106.8 (s, 1C), 50.1 (s, 1C), 35.0 (s, 1C), 32.2 (s, 2C), 29.0 (s, 1C), 26.2 (s, 1C). ¹⁹F NMR (376 MHz, DMSO-d₆) δ −139.2 (m, 1F), −142.2 (m, 1F).

### Example 6

### (±)-12-(3,4-difluorophenyl)-3,9,9-trimethyl-8,9,10,12-tetrahydrobenzo[b][4,7]phenanthrolin-11(7H)-one (Compound A81)

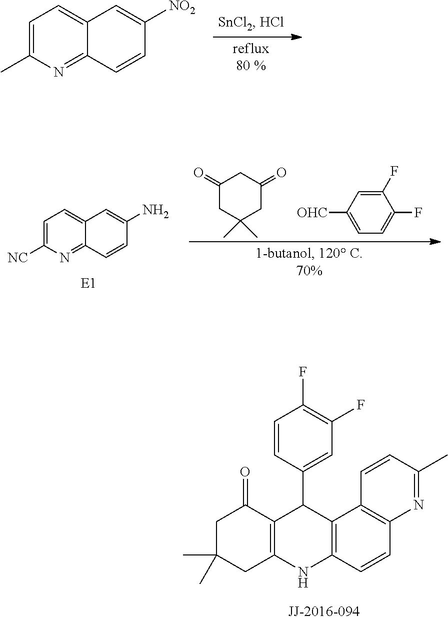

To a solution of 2-methyl-6-nitroquinoline (500 mg, 2.7 mmol) in 1N HCl (20 mL) was added a solution of SnCl2 (2.5 g, 13 mmol) in 1N HCl (10 mL) at RT. The resulting reaction mixture was reflux for 20 min and then cooled to RT. To the solution was added NaHCO₃ slowly until pH=10, and then extracted with DCM. The organic layer was washed with saturated NaHCO₃ aqueous solution and brine, dried over Na₂SO₄, concentrated, and purified on silica gel flash chromatography (DCM:MeOH=15:1), giving the E1 (336 mg, 80% yield).

Then JJ-2016-094 was synthesized in 70% yield from E1 following the representative procedure.

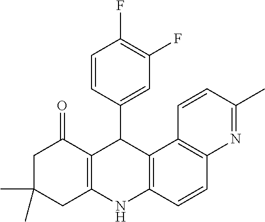

¹H NMR (400 MHz, DMSO-d₆) δ 9.81 (s, 1H), 8.23 (d, J=8.8 Hz, 1H), 7.76 (d, J=9.2 Hz, 1H), 7.46 (d, J=12.8 Hz, 1H), 7.24 (m, 2H), 7.14 (q, J=8.8 Hz, 1H), 6.91 (m, 1H), 5.77 (s, 1H), 2.52 (d, J=16.4 Hz, 1H), 2.51 (s, 3H), 2.37 (d, J=16.4 Hz, 1H), 2.19 (d, J=16.0 Hz, 1H), 2.00 (d, J=16.0 Hz, 1H), 0.99 (s, 3H), 0.81 (s, 3H). ¹³C NMR (100 MHz, DMSO-d₆) δ 193.2 (s, 1C), 155.9 (s, 1C), 151.0 (s, 1C), 148.8 (dd, J=244 and 13 Hz, 1C), 147.4 (dd, J=242 and 13 Hz, 1C), 145.1 (s, 1C), 144.5 (t, J=4 Hz, 1C), 133.8 (s, 1C), 131.0 (s, 1C), 128.6 (s, 1C), 124.5 (s, 1C), 124.0 (q, J=3 Hz, 1C), 122.5 (s, 1C), 120.2 (s, 1C), 116.9 (d, J=17 Hz, 1C), 116.2 (d, J=17 Hz, 1C), 115.6 (s, 1C), 106.2 (s, 1C), 50.2 (s, 1C), 40.0 (s, 1C), 35.0 (s, 1C), 32.1 (s, 1C), 29.1 (s, 1C), 26.2 (s, 1C), 24.3 (s, 1C). ¹⁹F NMR (376 MHz, DMSO-d₆) δ −139.5 (m, 1F), −142.6 (m, 1F).

### Example 7

### (±)-12-(3,4-difluorophenyl)-9,9-dimethyl-8,9,10,12-tetrahydrobenzo[b][4,7]phenanthroline-11(7H)-thione (Compound A82)

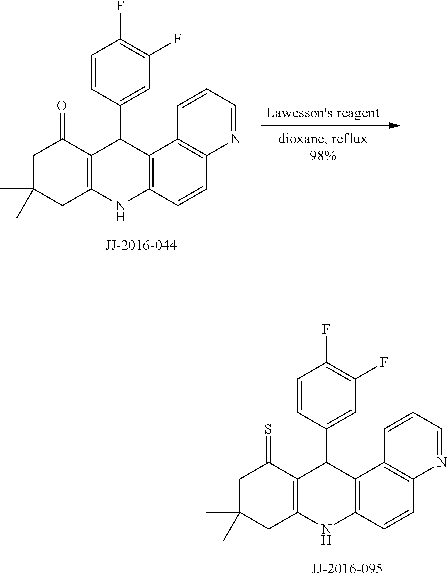 [SMILES: CC1(C)CC(=O)C2=C(C1)Nc1ccc3ncccc3c1C2c1ccc(F)c(F)c1 ; CC1(C)CC(=S)C2=C(C1)Nc1ccc3ncccc3c1C2c1ccc(F)c(F)c1]

A solution of JJ-2016-044 (39 mg, 0.1 mmol) and Lawesson's reagent (24 mg, 0.06 mmol) in dioxane (2 mL) was reflux for 4 hours and then cooled to RT. The mixture was diluted with EtOAc (60 mL), washed with saturated NaHCO₃ aqueous solution and brine, dried over Na₂SO₄, concentrated, and purified on silica gel flash chromatography (DCM:MeOH=15:1), giving the JJ-2016-095 (43 mg, 98% yield).

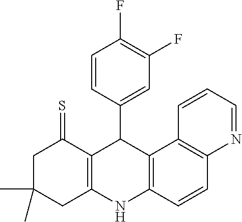 [SMILES: CC1(C)CC(=S)C2=C(C1)Nc1ccc3ncccc3c1C2c1ccc(F)c(F)c1]

¹H NMR (400 MHz, DMSO-d₆) δ 10.9 (s, 1H), 8.75 (dd, J=8.8 and 1.6 Hz, 1H), 8.53 (d, J=8.4 Hz, 1H), 7.94 (d, J=9.2 Hz, 1H), 7.65 (d, J=9.2 Hz, 1H), 7.50 (dd, J=8.8 and 4.0 Hz, 1H), 7.42 (ddd, J=12.0, 8.0 and 2.0 Hz, 1H), 7.17 (dt, J=10.4 and 8.8 Hz, 1H), 7.10 (m, 1H), 6.52 (s, 1H), 2.80 (d, J=16.8 Hz, 1H), 2.75 (d, J=16.8 Hz, 1H), 2.68 (d, J=16.8 Hz, 1H), 2.59 (d, J=16.8 Hz, 1H), 1.03 (s, 3H), 0.83 (s, 3H). ¹³C NMR (100 MHz, DMSO-d₆) δ 219.2 (s, 1C), 150.1 (s, 1C), 148.7 (dd, J=244 and 12 Hz, 1C), 148.5 (s, 1C), 147.5 (dd, J=243 and 13 Hz, 1C), 145.9 (s, 1C), 142.8 (t, J=4 Hz, 1C), 132.6 (s, 1C), 130.8 (s, 1C), 129.5 (s, 1C), 125.9 (s, 1C), 124.7 (q, J=3 Hz, 1H), 122.2 (s, 1C), 120.6 (s, 1C), 118.9 (s, 1C), 117.4 (s, 1C), 117.1 (d, J=17 Hz, 1C), 116.7 (d, J=17 Hz, 1C), 59.2 (s, 1C), 41.4 (s, 1C), 38.2 (s, 1C), 32.3 (s, 1C), 28.5 (s, 1C), 25.5 (s, 1C). 19F NMR (376 MHz, DMSO-d₆) δ −139.5 (m, 1F), −142.0 (m, 1F).

### Example 8

### (±)-12-(3,4-difluorophenyl)-9,9-dimethyl-8,9,10,12-tetrahydropyrazino[2,3-a]acridin-11(7H)-one (Compound A83)

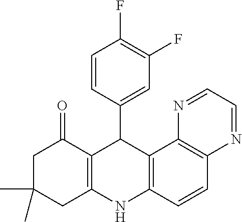 [SMILES: CC1(C)CC(=O)C2=C(C1)Nc1ccc3nccnc3c1C2c1ccc(F)c(F)c1]

¹H NMR (400 MHz, DMSO-d₆) δ 10.0 (s, 1H), 8.83 (d, J=2.0 Hz, 1H), 8.71 (d, J=2.0 Hz, 1H), 7.93 (d, J=9.2 Hz, 1H), 7.62 (d, J=9.2 Hz, 1H), 7.16 (m, 2H), 6.96 (m, 1H), 6.13 (s, 1H), 2.59 (d, J=16.8 Hz, 1H), 2.48 (d, J=16.8 Hz, 1H), 2.25 (d, J=16.0 Hz, 1H), 2.09 (d, J=16.0 Hz, 1H), 1.05 (s, 3H), 0.93 (s, 3H). ¹³C NMR (100 MHz, DMSO-d₆) δ 193.7 (s, 1C), 151.4 (s, 1C), 148.8 (dd, J=244 and 13 Hz, 1C), 147.4 (dd, J=242 and 13 Hz, 1C), 145.0 (s, 1C), 144.8 (t, J=4 Hz, 1C), 142.8 (s, 1C), 140.7 (s, 1C), 139.8 (s, 1C), 137.5 (s, 1C), 128.8 (s, 1C), 123.9 (q, J=3 Hz, 1C), 121.2 (s, 1C), 117.2 (s, 1C), 116.7 (d, J=17 Hz, 1C), 116.1 (d, J=17 Hz, 1C), 107.0 (s, 1C), 50.2 (s, 1C), 40.0 (s, 1C), 33.5 (s, 1C), 32.2 (s, 1C), 29.1 (s, 1C), 26.5 (s, 1C). ¹⁹F NMR (376 MHz, DMSO-d₆) δ −139.6 (m, 1F), −142.7 (m, 1F).

### Example 9

### (±)-12-(3,4-difluorophenyl)-2,3,9,9-tetramethyl-8,9,10,12-tetrahydropyrazino[2,3-a]acridin-11(7H)-one (Compound A84)

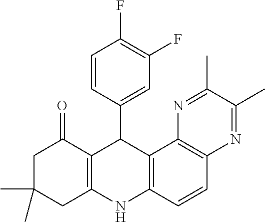

¹H NMR (400 MHz, DMSO-d₆) δ 9.87 (s, 1H), 7.75 (d, J=8.8 Hz, 1H), 7.43 (d, J=8.8 Hz, 1H), 7.16 (m, 2H), 7.01 (m, 1H), 6.07 (s, 1H), 2.62 (s, 3H), 2.56 (d, J=16.4 Hz, 1H), 2.55 (s, 3H), 2.46 (d, J=16.4 Hz, 1H), 2.22 (d, J=16.0 Hz, 1H), 2.05 (d, J=16.0 Hz, 1H), 1.03 (s, 3H), 0.93 (s, 3H). ¹³C NMR (100 MHz, DMSO-d₆) δ 193.6 (s, 1C), 153.1 (s, 1C), 151.6 (s, 1C), 150.9 (s, 1C), 148.7 (dd, J=244 and 13 Hz, 1C), 147.3 (dd, J=242 and 13 Hz, 1C), 145.2 (t, J=4 Hz, 1C), 138.5 (s, 1C), 137.6 (s, 1C), 136.2 (s, 1C), 127.5 (s, 1C), 124.0 (q, J=3 Hz, 1C), 119.4 (s, 1C), 117.2 (s, 1C), 116.5 (d, J=17 Hz, 1C), 116.3 (d, J=17 Hz, 1C), 106.7 (s, 1C), 50.2 (s, 1C), 40.1 (s, 1C), 33.7 (s, 1C), 32.2 (s, 1C), 29.1 (s, 1C), 26.5 (s, 1C), 23.0 (s, 1C), 22.3 (s, 1C). ¹⁹F NMR (376 MHz, DMSO-d₆) δ −140.0 (m, 1F), −143.0 (m, 1F).

### Example 10

### (±)-14-(3,4-difluorophenyl)naphtho[2,3-b][4,7]phenanthroline-8,13(7H,14H)-dione (Compound A85)

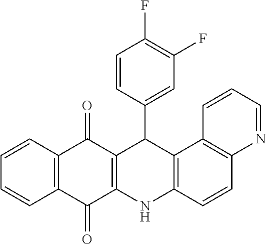 [SMILES: O=C1C2=C(C(=O)c3ccccc31)C(c1ccc(F)c(F)c1)c1c(ccc3ncccc13)N2]

¹H NMR (400 MHz, DMSO-d₆) δ 10.60 (s, 1H), 8.76 (d, J=4.0 Hz, 1H), 8.51 (d, J=8.8 Hz, 1H), 8.44 (d, J=8.0 Hz, 1H), 8.00 (m, 3H), 7.91 (t, J=8.0 Hz, 1H), 7.68 (t, J=7.6 Hz, 1H), 7.43 (m, 2H), 7.19 (q, J=8.8 Hz, 1H), 7.06 (m, 1H), 6.06 (s, 1H). ¹³C NMR (100 MHz, DMSO-d₆) δ 179.3 (s, 1C), 175.1 (s, 1C), 149.0 (dd, J=245 and 13 Hz, 1C), 148.8 (s, 1C), 147.9 (dd, J=243 and 13 Hz, 1C), 146.1 (s, 1C), 144.7 (s, 1C), 142.9 (t, J=4 Hz, 1C), 134.4 (s, 1C), 133.5 (s, 1C), 131.4 (s, 1C), 131.2 (1C), 130.6 (s, 1C), 129.8 (s, 1C), 128.3 (s, 1C), 125.8 (s, 1C), 124.5 (q, J=3 Hz, 1C), 124.1 (s, 1C), 122.2 (s, 1C), 121.5 (s, 1C), 117.2 (d, J=17 Hz, 1C), 116.9 (d, J=17 Hz, 1C), 116.7 (s, 1C), 110.0 (s, 1C), 35.4 (s, 1C). ¹⁹F NMR (376 MHz, DMSO-d₆) δ −138.8 (m, 1F), −141.6 (m, 1F).

### Example 11

### (±)-12-(3,4-difluorophenyl)-9,9-dimethyl-8,9,10,12-tetrahydropyrimido[5,4-a]acridin-11(7H)-one (Compound A86)

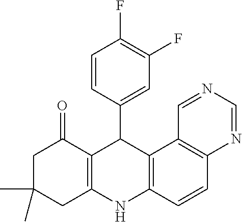 [SMILES: CC1(C)CC(=O)C2=C(C1)Nc1ccc3ncncc3c1C2c1ccc(F)c(F)c1]

¹H NMR (400 MHz, DMSO-d₆) δ 10.04 (s, 1H), 9.65 (s, 1H), 9.10 (s, 1H), 7.89 (d, J=8.8 Hz, 1H), 7.77 (d, J=8.8 Hz, 1H), 7.39 (ddd, J=11.2, 8.0 and 1.6 Hz, 1H), 7.20 (q, J=8.4 Hz, 1H), 7.03 (m, 1H), 6.01 (s, 1H), 2.57 (d, J=16.8 Hz, 1H), 2.42 (d, J=16.8 Hz, 1H), 2.24 (d, J=16.0 Hz, 1H), 2.06 (d, J=16.0 Hz, 1H), 1.04 (s, 3H), 0.86 (s, 3H). ¹³C NMR (100 MHz, DMSO-d₆) δ 193.4 (s, 1C), 155.5 (s, 1C), 152.8 (s, 1C), 150.7 (s, 1C), 148.9 (dd, J=244 and 13 Hz, 1C), 147.6 (dd, J=243 and 13 Hz, 1C), 147.4 (s, 1C), 144.4 (t, J=4 Hz, 1C), 135.4 (s, 1C), 128.3 (s, 1C), 125.5 (s, 1C), 124.2 (q, J=3 Hz, 1C), 122.9 (s, 1C), 117.1 (d, J=17 Hz, 1C), 116.5 (d, J=17 Hz, 1C), 115.6 (s, 1C), 106.4 (s, 1C), 50.2 (s, 1C), 40.0 (s, 1C), 34.5 (s, 1C), 32.1 (s, 1C), 29.0 (s, 1C), 26.3 (s, 1C). 19F NMR (376 MHz, DMSO-d₆) δ −139.2 (m, 1F), −142.0 (m, 1F).

### Example 12

### (±)-12-(3,4-difluorophenyl)-4,9,9-trimethyl-11-oxo-7,8,9,10,11,12-hexahydrobenzo[b][4,7]phenanthrolin-4-ium iodide (Compound A87)

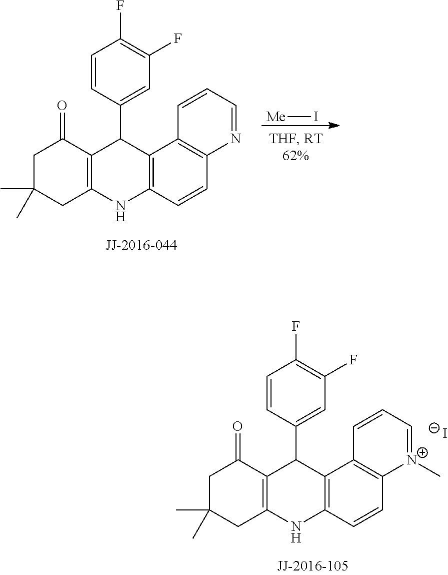

A solution of JJ-2016-044 (29 mg, 0.2 mmol) and methyl iodide (19 μL, 0.3 mmol) in THF (2 mL) was stirred for 4 days at RT. The precipitate was filtered and washed with Et₂O giving the JJ-2016-105 (66 mg, 62% yield).

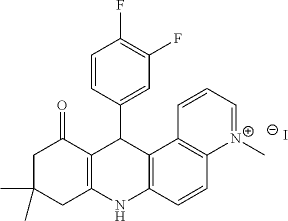

¹H NMR (400 MHz, DMSO-d₆) δ 10.29 (s, 1H), 9.26 (d, J=8.8 Hz, 1H), 9.22 (d, J=5.6 Hz, 1H), 8.42 (d, J=9.6 Hz, 1H), 8.00 (d, J=8.8 Hz, 1H), 7.98 (d, J=8.8 Hz, 1H), 7.38 (ddd, J=11.2, 8.0 and 2.4 Hz, 1H), 7.23 (q, J=8.4 Hz, 1H), 7.02 (m, 1H), 6.00 (s, 1H), 4.56 (s, 3H), 2.61 (d, J=17.2 Hz, 1H), 2.46 (d, J=17.2 Hz, 1H), 2.28 (d, J=16.0 Hz, 1H), 2.09 (d, J=16.0 Hz, 1H), 1.05 (s, 3H), 0.86 (s, 3H). ¹³C NMR (100 MHz, DMSO-d₆) δ 193.7 (s, 1C), 150.4 (s, 1C), 148.9 (dd, J=245 and 13 Hz, 1C), 147.7 (dd, J=243 and 13 Hz, 1C), 146.6 (s, 1C), 143.5 (t, J=4 Hz, 1C), 137.3 (s, 1C), 135.9 (s, 1C), 128.3 (s, 1C), 125.7 (s, 1C), 124.2 (q, J=3 Hz, 1C), 122.4 (s, 1C), 119.5 (s, 1C), 117.3 (d, J=17 Hz, 1C), 116.7 (s, 1C), 116.5 (d, J=17 Hz, 1C), 107.0 (s, 1C), 50.1 (s, 1C), 45.7 (s, 1C), 39.8 (s, 1C), 35.0 (s, 1C), 32.2 (s, 1C), 28.9 (s, 1C), 26.2 (s, 1C). 19F NMR (376 MHz, DMSO-d₆) δ −138.9 (m, 1F), −141.8 (m, 1F).

### Example 13

### (±)-12-(2,4-dichlorophenyl)-9,9-dimethyl-8,9,10,12-tetrahydrobenzo[b][4,7]phenanthrolin-11(7H)-one (Compound A88)

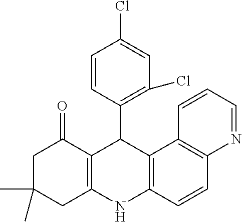 [SMILES: CC1(C)CC(=O)C2=C(C1)Nc1ccc3ncccc3c1C2c1ccc(Cl)cc1Cl]

¹H NMR (400 MHz, DMSO-d₆) δ 9.94 (s, 1H), 8.66 (d, J=4.0 Hz, 1H), 8.39 (d, J=8.4 Hz, 1H), 7.88 (d, J=9.2 Hz, 1H), 7.51 (d, J=8.8 Hz, 1H), 7.42 (dd, J=8.4 and 4.0 Hz, 1H), 7.38 (d, J=2.0 Hz, 1H), 7.35 (d, J=8.4 Hz, 1H), 7.21 (dd, J=8.4 and 2.0 Hz, 1H), 5.99 (s, 1H), 2.58 (d, J=16.8 Hz, 1H), 2.40 (d, J=16.8 Hz, 1H), 2.22 (d, J=16.0 Hz, 1H), 1.98 (d, J=16.0 Hz, 1H), 1.04 (s, 3H), 0.87 (s, 3H). ¹³C NMR (100 MHz, DMSO-d₆) δ 193.0, 150.9, 147.6, 145.4, 143.5, 134.7, 132.8, 131.5, 131.1, 130.2, 129.6, 128.4, 127.5, 126.7, 121.7, 120.7, 115.6, 106.3, 50.2, 34.1, 32.0, 29.1, 26.2.

### Example 14

### (±)-12-(3,4-difluorophenyl)-9,9-dimethyl-9,10-dihydro-8H-benzo[a]xanthen-11(12H)-one (Compound A89)

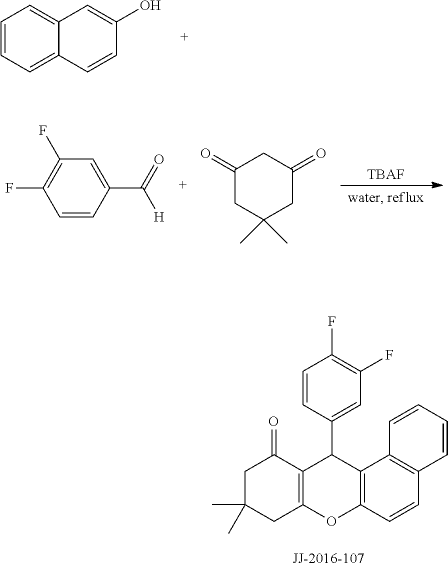

To a mixture of 2-naphthol (144 mg, 1 mmol), 3,4-difluorobenzaldehyde (111 μL, 1 mmol) and dimedone (140 mg, 1 mmol) in water (3 mL) was added TBAF (0.1 mL, 1M in THF, 0.1 mmol). The resulting reaction mixture was stirred for 4 hours and then cooled to RT, filtered. The collected solid was recrystallized from Et2O/hexane to afford the JJ-2016-107 (173 mg, 44% yield).

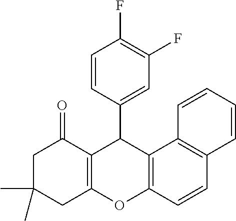 [SMILES: CC1(C)CC(=O)C2=C(C1)Oc1ccc3ccccc3c1C2c1ccc(F)c(F)c1]

¹H NMR (400 MHz, CDCl₃) δ 7.87 (d, J=8.4 Hz, 1H), 7.80 (d, J=8.4 Hz, 1H), 7.78 (d, J=9.2 Hz, 1H), 7.45 (t, J=7.6 Hz, 1H), 7.40 (t, J=7.6 Hz, 1H), 7.32 (d, J=9.2 Hz, 1H), 7.08 (m, 2H), 6.94 (q, J=8.4 Hz, 1H), 5.68 (s, 1H), 2.57 (s, 2H), 2.28 (dd, J=25.6 and 16.4 Hz, 2H), 1.12 (s, 3H), 0.97 (s, 3H). ¹³C NMR (100 MHz, CDCl₃) δ 196.8 (s, 1C), 164.2 (s, 1C), 150.1 (dd, J=247 and 13 Hz, 1C), 148.8 (dd, J=246 and 13 Hz, 1C), 147.8 (s, 1C), 141.6 (t, J=4 Hz, 1C), 131.5 (s, 1C), 131.1 (s, 1C), 129.3 (s, 1C), 128.5 (s, 1C), 127.2 (s, 1C), 125.1 (s, 1C), 124.3 (q, J=3 Hz, 1C), 123.3 (s, 1C), 117.2 (d, J=17 Hz, 1C), 117.0 (s, 1C), 116.7 (d, J=17 Hz, 1C), 116.6 (s, 1C), 113.6 (s, 1C), 50.8 (s, 1C), 41.4 (s, 1C), 33.9 (s, 1C), 32.2 (s, 1C), 29.3 (s, 1C), 27.1 (s, 1C). 19F NMR (376 MHz, CDCl₃) δ −137.9 (m, 1F), −141.4 (m, 1F).

### Example 15

### (±)-9-(3,4-difluorophenyl)-3,3-dimethyl-6-(pyrrolidin-1-yl)-3,4,9,10-tetrahydroacridin-1(2H)-one (Compound A90)

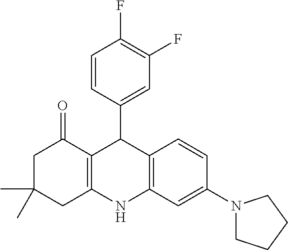 [SMILES: CC1(C)CC(=O)C2=C(C1)Nc1cc(N3CCCC3)ccc1C2c1ccc(F)c(F)c1]

¹H NMR (400 MHz, DMSO-d₆) δ 9.31 (s, 1H), 7.19 (q, J=8.4 Hz, 1H), 7.08 (ddd, J=11.2, 8.0 and 1.6 Hz, 1H), 6.91 (m, 1H), 6.87 (d, J=8.4 Hz, 1H), 6.13 (dd, J=8.4 and 2.0 Hz, 1H), 6.10 (d, J=1.6 Hz, 1H), 4.97 (s, 1H), 3.15 (m, 4H), 2.46 (d, J=16.8 Hz, 1H), 2.38 (d, J=16.8 Hz, 1H), 2.16 (d, J=16.0 Hz, 1H), 2.99 (d, J=16.0 Hz, 1H), 1.91 (m, 4H), 1.02 (s, 3H), 0.92 (s, 3H). ¹³C NMR (100 MHz, DMSO-d₆) δ 192.7 (s, 1C), 152.0 (s, 1C), 148.9 (dd, J=244 and 13 Hz, 1C), 147.3 (dd, J=242 and 13 Hz, 1C), 147.2 (t, J=4 Hz, 1C), 146.8 (s, 1C), 136.5 (s, 1C), 130.0 (s, 1C), 123.2 (dd, J=6 and 3 Hz, 1C), 116.7 (d, J=17 Hz, 1C), 115.4 (d, J=17 Hz, 1C), 112.2 (s, 1C), 107.6 (s, 1C), 106.2 (s, 1C), 97.8 (s, 1C), 50.2 (s, 1C), 47.2 (s, 1C), 40.4 (s, 1C), 38.3 (s, 1C), 32.0 (s, 1C), 29.0 (s, 1C), 26.7 (s, 1C), 24.9 (s, 1C). 19F NMR (376 MHz, DMSO-d₆) δ −139.8 (m, 1F), −143.6 (m, 1F).

### Example 16

### (±)-12-(3,4-difluorophenyl)-9,9-dimethyl-8,9,10,12-tetrahydrobenzo[j][3,7]phenanthrolin-11(7H)-one (Compound A91)

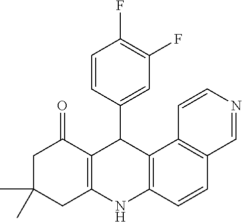 [SMILES: CC1(C)CC(=O)C2=C(C1)Nc1ccc3cnccc3c1C2c1ccc(F)c(F)c1]

¹H NMR (400 MHz, DMSO-d₆) δ 9.98 (s, 1H), 7.98 (d, J=8.4 Hz, 1H), 7.44 (d, J=8.4 Hz, 1H), 7.29 (ddd, J=11.2, 8.0 and 1.2 Hz, 1H), 7.18 (dt, J=11.2 and 8.4 Hz, 1H), 6.97 m, 1H), 5.76 (s, 1H), 2.56 (d, J=16.8 Hz, 1H), 2.42 (d, J=16.8 Hz, 1H), 2.24 (d, J=16.0 Hz, 1H), 2.06 (d, J=16.0 Hz, 1H), 1.03 (s, 3H), 0.85 (s, 3H). ¹³C NMR (100 MHz, DMSO-d₆) δ 193.5 (s, 1C), 150.8 (s, 1C), 148.8 (dd, J=244 and 13 Hz, 1C), 147.5 (dd, J=243 and 13 Hz, 1C), 144.2 (t, J=15.2 Hz, 1C), 137.7 (s, 1C), 128.4 (s, 1C), 124.0 (q, J=12.8 Hz, 1C), 118.6 (s, 1C), 116.9 (d, J=17 Hz, 1C), 16.3 (d, J=17 Hz, 1C), 114.3 (s, 1C), 107.2 (s, 1C), 50.1 (s, 1C), 40.0 (s, 1C), 34.8 (s, 1C), 32.1 (s, 1C), 29.0 (s, 1C), 26.2 (s, 1C). 19F NMR (376 MHz, DMSO-d₆) δ −139.4 (m, 1F), −142.3 (m, 1F).

### Example 17

### (±)-12-(benzo[b]thiophen-2-yl)-9,9-dimethyl-8,9,10,12-tetrahydrobenzo[b][4,7]phenanthrolin-11(7H)-one (Compound A92)

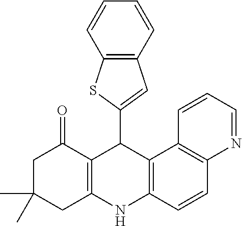 [SMILES: CC1(C)CC(=O)C2=C(C1)Nc1ccc3ncccc3c1C2c1cc2ccccc2s1]

¹H NMR (400 MHz, DMSO-d₆) δ 10.04 (s, 1H), 8.71 (dd, J=4.4 and 1.2 Hz, 1H), 8.46 (d, J=8.4 Hz, 1H), 7.95 (d, J=8.8 Hz, 1H), 7.70 (d, J=7.6 Hz, 1H), 7.59 (d, J=6.4 Hz, 1H), 7.57 (d, J=8.8 Hz, 1H), 7.44 (dd, J=8.4 and 4.0 Hz, 1H), 7.17 (m, 2H), 6.91 (s, 1H), 6.24 (s, 1H), 2.58 (d, J=16.8 Hz, 1H), 2.43 (d, J=16.8 Hz, 1H), 2.29 (d, J=16.0 Hz, 1H), 2.14 (d, J=16.0 Hz, 1H), 1.05 (s, 3H), 0.94 (s, 3H). ¹³C NMR (100 MHz, DMSO-d₆) δ 193.3, 151.3, 150.8, 147.8, 145.4, 139.0, 138.8, 134.5, 130.6, 129.5, 126.6, 124.0, 123.5, 123.1, 122.2, 122.0, 120.4, 119.7, 114.9, 105.4, 50.2, 40.0, 32.1, 31.3, 29.2, 26.5.

### Example 18

### (±)-12-(benzo[b]thiophen-2-yl)-9,9-dimethyl-8,9,10,12-tetrahydropyrazino[2,3-a]acridin-11(7H)-one (Compound A93)

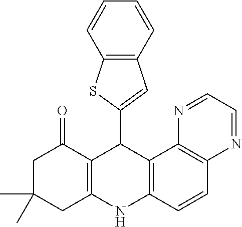 [SMILES: CC1(C)CC(=O)C2=C(C1)Nc1ccc3nccnc3c1C2c1cc2ccccc2s1]

¹H NMR (400 MHz, DMSO-d₆) δ 10.17 (s, 1H), 8.90 (s, 1H), 8.77 (s, 1H), 7.98 (d, J=9.2 Hz, 1H), 7.70 (d, J=7.6 Hz, 1H), 7.64 (d, J=9.2 Hz, 1H), 7.6 Hz, 1H), 7.16 (m, 2H), 6.80 (s, 1H), 6.53 (s, 1H), 2.61 (d, J=16.4 Hz, 1H), 2.52 (d, J=16.4 Hz, 1H), 2.31 (d, J=16.0 Hz, 1H), 2.18 (d, J=16.0 Hz, 1H), 1.07 (s, 3H), 1.03 (s, 3H). ¹³C NMR (100 MHz, DMSO-d₆) δ 193.7, 151.8, 151.4, 145.1, 142.9, 140.8, 139.7, 139.2, 138.6, 137.7, 128.9, 123.9, 123.4, 122.9, 122.1, 121.1, 119.4, 116.6, 106.3, 50.2, 40.0, 32.2, 29.7, 29.1, 26.8.

### Example 19

### (±)-9-(benzo[b]thiophen-2-yl)-3,3-dimethyl-6-(pyrrolidin-1-yl)-3,4,9,10-tetrahydroacridin-1(2H)-one (Compound A94)

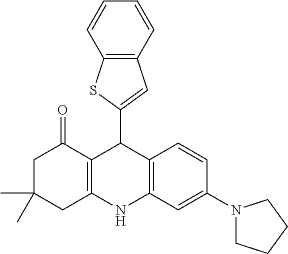 [SMILES: CC1(C)CC(=O)C2=C(C1)Nc1cc(N3CCCC3)ccc1C2c1cc2ccccc2s1]

¹H NMR (400 MHz, DMSO-d₆) δ 9.40 (s, 1H), 7.72 (d, J=8.0 Hz, 1H), 7.63 (d, J=8.0 Hz, 1H), 7.24 (td, J=8.0 and 1.2 Hz, 1H), 7.17 (td, J=8.0 and 1.2 Hz, 1H), 7.04 (d, J=8.4 Hz, 1H), 6.91 (s, 1H), 6.21 (dd, J=8.4 and 2.4 Hz, 1H), 6.11 (d, J=2.4 Hz, 1H), 5.34 (s, 1H), 3.18 (m, 4H), 2.49 (d, J=16.8 Hz, 1H), 2.39 (d, J=16.8 Hz, 1H), 2.21 (d, J=16.0 Hz, 1H), 2.05 (d, J=16.0 Hz, 1H), 1.93 (m, 4H), 1.04 (s, 3H), 1.00 (s, 3H). ¹³C NMR (100 MHz, DMSO-d₆) δ 192.6, 154.8, 152.1, 147.0, 139.5, 138.6, 136.6, 130.1, 123.9, 123.1, 122.8, 122.1, 118.1, 111.5, 107.5, 105.8, 97.8, 50.2, 47.3, 40.4, 34.8, 31.9, 29.2, 26.8, 24.9.

### Example 20

### (±)-12-(benzo[b]thiophen-2-yl)-9,9-dimethyl-8,9,10,12-tetrahydrobenzo[a]acridin-11(7H)-one (Compound A95)

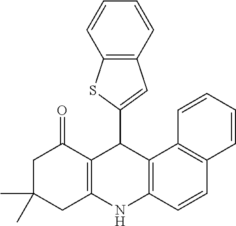 [SMILES: CC1(C)CC(=O)C2=C(C1)Nc1ccc3ccccc3c1C2c1cc2ccccc2s1]

¹H NMR (400 MHz, DMSO-d₆) δ 9.91 (s, 1H), 8.02 (d, J=8.4 Hz, 1H), 7.84 (d, J=8.4 Hz, 1H), 7.70 (d, J=8.0 Hz, 1H), 7.58 (d, J=7.6, 1H), 7.47 (t, J=7.6 Hz, 1H), 7.34 (t, J=9.2 Hz, 2H), 7.18 (m, 2H), 6.87 (s, 1H), 6.22 (s, 1H), 2.57 (d, J=16.8 Hz, 1H), 2.42 (d, J=16.8 Hz, 1H), 2.28 (d, J=16.0 Hz, 1H), 2.12 (d, J=16.0 Hz, 1H), 1.05 (s, 3H), 0.93 (s, 3H). ¹³C NMR (100 MHz, DMSO-d₆) δ 193.1, 151.4, 151.1, 139.1, 138.7, 134.3, 131.3, 130.3, 128.5, 127.1, 123.9, 123.8, 123.4, 123.0, 122.2, 122.1, 119.5, 117.0, 114.9, 105.6, 50.2, 40.0, 32.1, 31.5, 29.2, 26.6.

### Example 21

### (±)-12-(3,4-difluorophenyl)-9,9-dimethyl-8,9,10,12-tetrahydrobenzo[j][1,7]phenanthrolin-11(7H)-one (Compound A96)

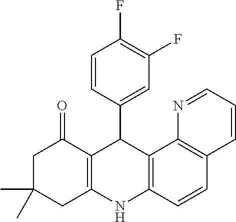 [SMILES: CC1(C)CC(=O)C2=C(C1)Nc1ccc3cccnc3c1C2c1ccc(F)c(F)c1]

¹H NMR (400 MHz, DMSO-d₆) δ 9.86 (s, 1H), 8.81 (dd, J=4.4 and 1.6 Hz, 1H), 8.20 (dd, J=8.0 and 1.6 Hz, 1H), 7.81 (d, J=8.4 Hz, 1H), 7.38 (d, J=8.8 Hz, 1H), 7.32 (dd, J=8.0 and 4.4 Hz, 1H), 7.18 (ddd, J=12.0, 8.0 and 2.0 Hz, 1H), 7.13 (dt, J=10.8 and 8.4 Hz, 1H), 6.99 (m, 1H), 6.21 (s, 1H), 2.58 (d, J=16.8 Hz, 1H), 2.48 (d, J=16.8 Hz, 1H), 2.24 (d, J=16.0 Hz, 1H), 2.08 (d, J=16.0 Hz, 1H), 1.04 (s, 3H), 0.93 (s, 3H). ¹³C NMR (100 MHz, DMSO-d₆) δ 193.5 (s, 1C), 151.6 (s, 1C), 150.2 (s, 1C), 148.7 (dd, J=243 and 13 Hz, 1C), 147.3 (dd, J=242 and 13 Hz, 1C), 145.7 (s, 1C), 145.3 (t, J=4 Hz, 1C), 137.3 (s, 1C), 136.0 (s, 1C), 127.7 (s, 1C), 124.7 (s, 1C), 123.9 (q, J=3 Hz, 1C), 119.3 (s, 1C), 117.5 (s, 2C), 116.5 (d, J=17 Hz, 1C), 116.1 (d, J=17 Hz, 1C), 107.2 (s, 1C), 50.3 (s, 1C), 40.1 (s, 1C), 33.5 (s, 1C), 32.2 (s, 1C), 29.1 (s, 1C), 26.5 (s, 1C). ¹⁹F NMR (376 MHz, DMSO-d₆) δ −139.9 (m, 1F), −143.1 (m, 1F).

### Example 22

### (±)-9-(5-chlorothiophen-2-yl)-3,3-dimethyl-6-(pyrrolidin-1-yl)-3,4,9,10-tetrahydroacridin-1(2H)-one (Compound A97)

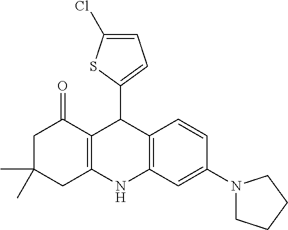 [SMILES: CC1(C)CC(=O)C2=C(C1)Nc1cc(N3CCCC3)ccc1C2c1ccc(Cl)s1]

¹H NMR (400 MHz, DMSO-d₆) δ 9.39 (s, 1H), 7.00 (d, J=8.8 Hz, 1H), 6.74 (d, J=4.0 Hz, 1H), 6.41 (d, J=4.0 Hz, 1H), 6.21 (dd, J=8.4 and 2.4 Hz, 1H), 6.09 (d, J=2.0 Hz, 1H), 5.16 (s, 1H), 3.18 (t, J=6.4 Hz, 4H), 2.44 (d, J=16.8 Hz, 1H), 2.33 (d, J=16.8 Hz, 1H), 2.19 (d, J=16.4 Hz, 1H), 2.05 (d, J=16.4 Hz, 1H), 1.92 (m, 4H), 1.03 (s, 3H), 0.99 (s, 3H). ¹³C NMR (100 MHz, DMSO-d₆) δ 192.7, 153.3, 152.2, 147.1, 136.6, 130.0, 125.8, 124.9, 121.6, 111.2, 107.6, 105.8, 97.7, 50.1, 47.3, 40.3, 34.3, 32.0, 29.2, 26.8, 24.9.

### Example 23

### (±)-9-(5-bromothiophen-2-yl)-3,3-dimethyl-6-(pyrrolidin-1l-yl)-3,4,9,10-tetrahydroacridin-1(2H)-one (Compound A98)

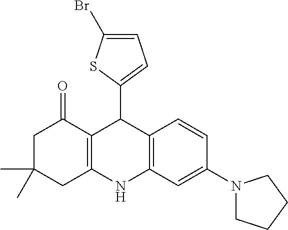 [SMILES: CC1(C)CC(=O)C2=C(C1)Nc1cc(N3CCCC3)ccc1C2c1ccc(Br)s1]

¹H NMR (400 MHz, DMSO-d₆) δ 9.39 (s, 1H), 7.00 (d, J=8.4 Hz, 1H), 6.85 (d, J=4.0 Hz, 1H), 6.40 Z(d, J=4.0 Hz, 1H), 6.21 (dd, J=8.4 and 2.4 Hz, 1H), 6.08 (d, J=2.0 Hz, 1H), 5.18 (s, 1H), 3.18 (t, J=6.4 Hz, 4H), 2.44 (d, J=16.8 Hz, 1H), 2.34 (d, J=16.8 Hz, 1H), 2.19 (d, J=16.0 Hz, 1H), 2.05 (d, J=16.0 Hz, 1H), 1.94 (m, 4H), 1.03 (s, 3H), 0.99 (m, 3H). ¹³C NMR (100 MHz, DMSO-d₆) δ 192.7, 155.9, 152.2, 147.1, 136.6, 130.0, 129.4, 122.7, 111.3, 108.0, 107.6, 105.9, 97.7, 50.1, 47.3, 40.3, 34.3, 32.0, 29.1, 26.8, 24.9.

### Example 24

### (±)-12-(5-chlorothiophen-2-yl)-9,9-dimethyl-8,9,10,12-tetrahydropyrazino[2,3-a]acridin-11(7H)-one (Compound A99)

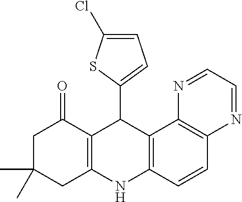 [SMILES: CC1(C)CC(=O)C2=C(C1)Nc1ccc3nccnc3c1C2c1ccc(Cl)s1]

¹H NMR (400 MHz, DMSO-d₆) δ 10.13 (s, 1H), 8.90 (d, J=1.6 Hz, 1H), 8.77 (d, J=1.6 Hz, 1H), 7.95 (d, J=8.8 Hz, 1H), 7.60 (d, J=8.8 Hz, 1H), 6.68 (d, J=3.6 Hz, 1H), 6.33 (d, J=3.6 Hz, 1H), 6.31 (s, 1H), 2.59 (d, J=17.2 Hz, 1H), 2.51 (d, J=17.2 Hz, 1H), 2.29 (d, J=16.0 Hz, 1H), 2.18 (d, J=16.0 Hz, 1H), 1.06 (s, 3H), 1.05 (s, 3H). ¹³C NMR (100 MHz, DMSO-d₆) δ 193.6, 151.9, 149.6, 145.1, 142.9, 140.5, 139.7, 137.4, 128.8, 125.8, 125.4, 122.7, 121.1, 116.4, 106.1, 50.1, 40.0, 32.2, 29.2, 29.0, 26.9.

### Example 25

### (±)-12-(5-bromothiophen-2-yl)-9,9-dimethyl-8,9,10,12-tetrahydropyrazino[2,3-a]acridin-11(7H)-one (Compound A100)

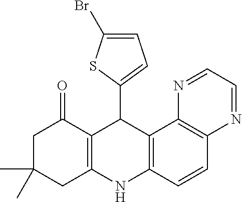 [SMILES: CC1(C)CC(=O)C2=C(C1)Nc1ccc3nccnc3c1C2c1ccc(Br)s1]

¹H NMR (400 MHz, DMSO-d₆) δ 10.12 (s, 1H), 8.89 (d, J=1.6 Hz, 1H), 8.77 (d, J=2.0 Hz, 1H), 7.95 (d, J=9.2 Hz, 1H), 7.59 (d, J=9.2 Hz, 1H), 6.79 (d, J=3.6 Hz, 1H), 6.33 (s, 1H), 6.32 (d, J=3.6 Hz, 1H), 2.59 (d, J=17.2 Hz, 1H), 2.52 (d, J=17.2 Hz, 1H), 2.28 (d, J=16.0 Hz, 1H), 2.18 (d, J=16.0 Hz, 1H), 1.06 (s, 3H), 1.05 (s, 3H). ¹³C NMR (100 MHz, DMSO-d₆) δ 193.6, 152.3, 151.9, 145.1, 142.9, 140.5, 139.7, 137.4, 129.3, 128.8, 123.8, 121.1, 116.5, 108.5, 106.1, 50.1, 40.0, 32.2, 29.3, 29.0, 26.9.

### Example 26

### (±)-12-(5-chlorothiophen-2-yl)-9,9-dimethyl-8,9,10,12-tetrahydropyrimido[5,4-a]acridin-11(7H)-one (Compound A101)

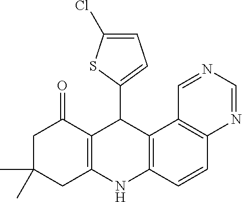 [SMILES: CC1(C)CC(=O)C2=C(C1)Nc1ccc3ncncc3c1C2c1ccc(Cl)s1]

¹H NMR (400 MHz, DMSO-d₆) δ 10.15 (s, 1H), 9.66 (s, 1H), 9.15 (s, 1H), 7.91 (d, J=9.2 Hz, 1H), 7.75 (d, J=9.2 Hz, 1H), 6.72 (d, J=4.0 Hz, 1H), 6.45 (d, J=4.0 Hz, 1H), 6.23 (s, 1H), 2.57 (d, J=16.8 Hz, 1H), 2.44 (d, J=16.8 Hz, 1H), 2.29 (d, J=16.4 Hz, 1H), 2.15 (d, J=16.4 Hz, 1H), 1.05 (s, 3H), 0.97 (s, 3H). ¹³C NMR (100 MHz, DMSO-d₆) δ 193.4, 155.4, 152.9, 151.1, 149.1, 147.3, 135.4, 128.4, 126.2, 125.8, 125.3, 123.5, 122.9, 114.5, 105.6, 50.1, 39.9, 32.2, 30.4, 29.0, 26.5.

### Example 27

### (±)-12-(5-bromothiophen-2-yl)-9,9-dimethyl-8,9,10,12-tetrahydropyrimido[5,4-a]acridin-11(7H)-one (Compound A102)

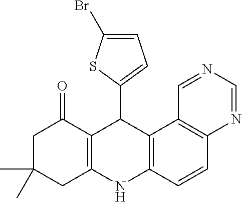 [SMILES: CC1(C)CC(=O)C2=C(C1)Nc1ccc3ncncc3c1C2c1ccc(Br)s1]

¹H NMR (400 MHz, DMSO-d₆) δ 10.14 (s, 1H), 9.66 (s, 1H), 9.15 (s, 1H), 7.91 (d, J=9.2 Hz, 1H), 7.74 (d, J=9.2 Hz, 1H), 6.83 (d, J=4.0 Hz, 1H), 6.43 (d, J=4.0 Hz, 1H), 6.25 (s, 1H), 2.56 (d, J=16.8 Hz, 1H), 2.44 (d, J=16.8 Hz, 1H), 2.28 (d, J=16.0 Hz, 1H), 2.15 (d, J=16.0 Hz, 1H), 1.05 (s, 3H), 0.97 (s, 3H). ¹³C NMR (100 MHz, DMSO-d₆) δ 193.4, 155.4, 152.9, 151.8, 151.1, 147.3, 135.3, 129.4, 128.4, 125.3, 124.6, 122.9, 114.6, 109.3, 105.7, 50.1, 39.9, 32.2, 30.3, 29.0, 26.5.

### Example 28

### (±)-9,9-dimethyl-12-(5-methylthiophen-2-yl)-8,9,10,12-tetrahydrobenzo[b][4,7]phenanthrolin-11(7H)-one (Compound A103)

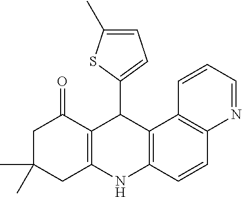 [SMILES: Cc1ccc(C2C3=C(CC(C)(C)CC3=O)Nc3ccc4ncccc4c32)s1]

¹H NMR (400 MHz, DMSO-d₆) δ 9.90 (s, 1H), 8.70 (dd, J=4.4 and 1.2 Hz, 1H), 8.41 (d, J=8.4 Hz, 1H), 7.89 (d, J=9.2 Hz, 1H), 7.51 (d, J=9.2 Hz, 1H), 7.45 (dd, J=8.4 and 4.0 Hz, 1H), 6.40 (d, J=3.2 Hz, 1H), 6.37 (dd, J=3.2 and 0.8 Hz, 2H), 6.03 (s, 1H), 2.54 (d, J=16.8 Hz, 1H), 2.42 (d, J=16.8 Hz, 1H), 2.25 (d, J=16.0 Hz, 1H), 2.12 (d, J=16.0 Hz, 1H), 1.04 (s, 3H), 0.98 (s, 3H). ¹³C NMR (100 MHz, DMSO-d₆) δ 193.2, 150.8, 147.9, 147.7, 145.4, 137.0, 134.2, 130.6, 129.1, 126.4, 124.2, 123.1, 121.8, 120.4, 115.8, 106.2, 50.2, 40.0, 32.1, 30.4, 29.2, 26.6, 14.8.

### Example 29

### (±)-9,9-dimethyl-12-(5-methylthiophen-2-yl)-8,9,10,12-tetrahydropyrazino[2,3-a]acridin-11(7H)-one (Compound A104)

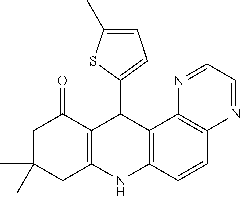 [SMILES: Cc1ccc(C2C3=C(CC(C)(C)CC3=O)Nc3ccc4nccnc4c32)s1]

¹H NMR (400 MHz, DMSO-d₆) δ 10.02 (s, 1H), 8.88 (d, J=2.0 Hz, 1H), 8.75 (d, J=2.0 Hz, 1H), 7.92 (d, J=8.8 Hz, 1H), 7.57 (d, J=8.8 Hz, 1H), 6.35 (m, 3H), 2.58 (d, J=16.8 Hz, 1H), 2.48 (d, J=16.8 Hz, 1H), 2.27 (d, J=16.0 Hz, 1H), 2.19 (s, 3H), 2.15 (d, J=16.0 Hz, 1H), 1.06 (s, 6H). ¹³C NMR (100 MHz, DMSO-d₆) δ 193.5, 151.4, 148.3, 144.9, 142.7, 140.7, 139.7, 137.4, 136.5, 128.4, 124.2, 122.7, 121.1, 117.7, 106.9, 50.3, 40.0, 32.2, 29.2, 28.7, 26.9, 14.8.

### Exemplary Compounds

The compounds presented in Table 1 were prepared in an analogous manner to those described in Examples 1-29, using appropriate starting materials and reaction conditions.

TABLE 1
Exemplary Compounds

| Cmpd | CdId | Structure |
|---|---|---|
| A1 | Control [DPM- 2011- 95] | 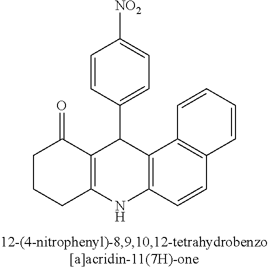 [SMILES: O=C1CCCC2=C1C(c1ccc([N+](=O)[O-])cc1)c1c(ccc3ccccc13)N2] |
| A2 | LO-171 [DPM- 2011- 97] | 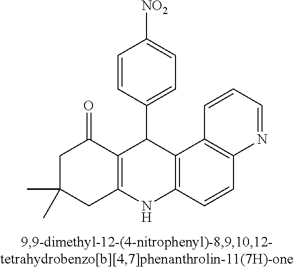 [SMILES: CC1(C)CC(=O)C2=C(C1)Nc1ccc3ncccc3c1C2c1ccc([N+](=O)[O-])cc1] |
| A3 | LO-36 | 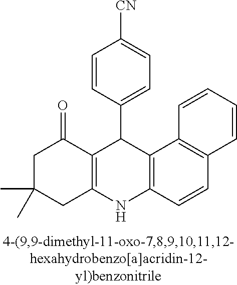 [SMILES: CC1(C)CC(=O)C2=C(C1)Nc1ccc3ccccc3c1C2c1ccc(C#N)cc1] |
| A4 | LO-8 | 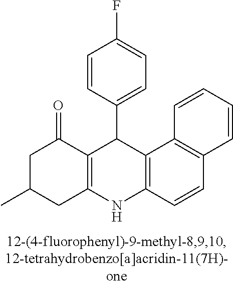 [SMILES: CC1CC(=O)C2=C(C1)Nc1ccc3ccccc3c1C2c1ccc(F)cc1] |
| A5 | LO-172 | 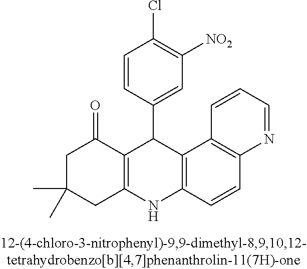 [SMILES: CC1(C)CC(=O)C2=C(C1)Nc1ccc3ncccc3c1C2c1ccc(Cl)c([N+](=O)[O-])c1] |
| A6 | LO-27 | 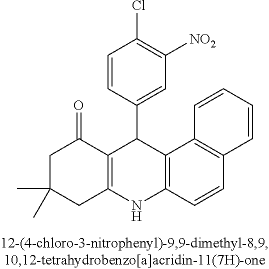 [SMILES: CC1(C)CC(=O)C2=C(C1)Nc1ccc3ccccc3c1C2c1ccc(Cl)c([N+](=O)[O-])c1] |
| A7 | LO-37 | 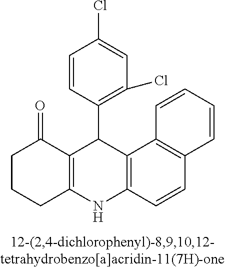 [SMILES: O=C1CCCC2=C1C(c1ccc(Cl)cc1Cl)c1c(ccc3ccccc13)N2] |
| A8 | LO-28 | 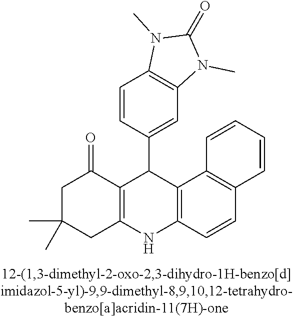 [SMILES: Cn1c(=O)n(C)c2cc(C3C4=C(CC(C)(C)CC4=O)Nc4ccc5ccccc5c43)ccc21] |
| A9 | LO-61 | 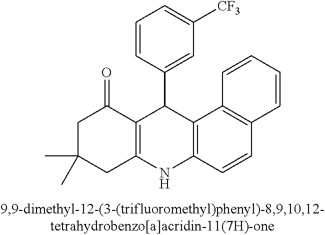 [SMILES: CC1(C)CC(=O)C2=C(C1)Nc1ccc3ccccc3c1C2c1cccc(C(F)(F)F)c1] |
| A10 | LO-67 | 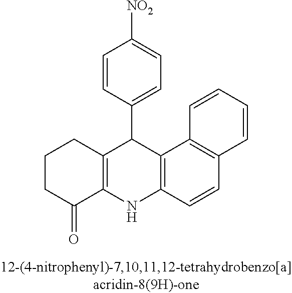 [SMILES: O=C1CCCC2=C1Nc1ccc3ccccc3c1C2c1ccc([N+](=O)[O-])cc1] |
| A12 | LO-42 | 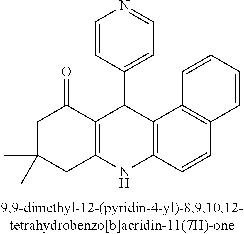 [SMILES: CC1(C)CC(=O)C2=C(C1)Nc1ccc3ccccc3c1C2c1ccncc1] |
| A13 | LO-4 | 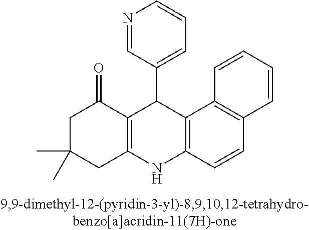 [SMILES: CC1(C)CC(=O)C2=C(C1)Nc1ccc3ccccc3c1C2c1cccnc1] |
| A14 | LO-54 | 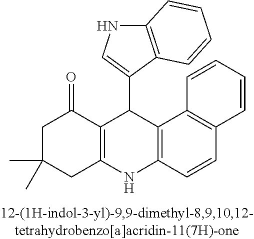 [SMILES: CC1(C)CC(=O)C2=C(C1)Nc1ccc3ccccc3c1C2c1c[nH]c2ccccc12] |
| A15 | LO-5 | 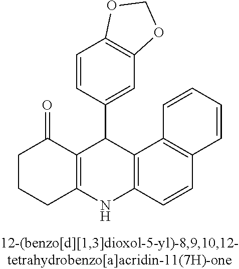 [SMILES: O=C1CCCC2=C1C(c1ccc3c(c1)OCO3)c1c(ccc3ccccc13)N2] |
| A16 | LO-53 | 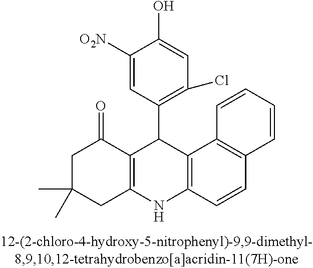 [SMILES: CC1(C)CC(=O)C2=C(C1)Nc1ccc3ccccc3c1C2c1cc([N+](=O)[O-])c(O)cc1Cl] |
| A17 | LO-43 | 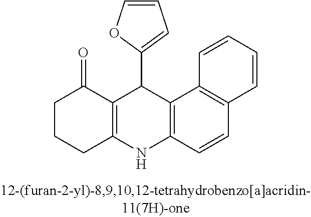 [SMILES: O=C1CCCC2=C1C(c1ccco1)c1c(ccc3ccccc13)N2] |
| A18 | LO-170 [DPM- 2011- 96] | 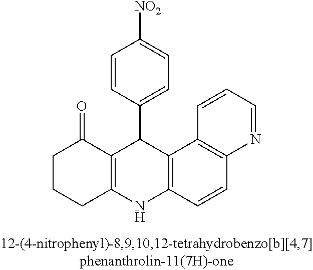 [SMILES: O=C1CCCC2=C1C(c1ccc([N+](=O)[O-])cc1)c1c(ccc3ncccc13)N2] |
| A19 | LO-146 | 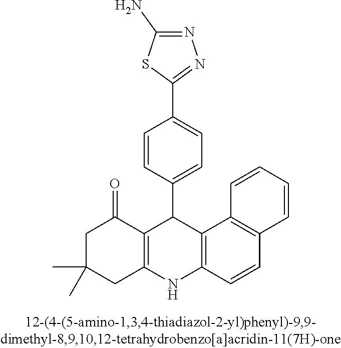 [SMILES: CC1(C)CC(=O)C2=C(C1)Nc1ccc3ccccc3c1C2c1ccc(-c2nnc(N)s2)cc1] |
| A20 | LO-7 | 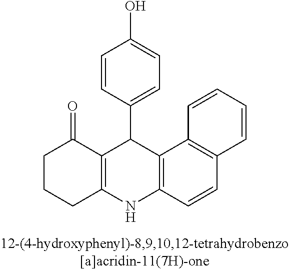 [SMILES: O=C1CCCC2=C1C(c1ccc(O)cc1)c1c(ccc3ccccc13)N2] |
| A21 | LO-6 | 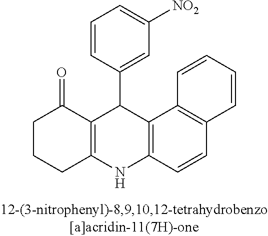 [SMILES: O=C1CCCC2=C1C(c1cccc([N+](=O)[O-])c1)c1c(ccc3ccccc13)N2] |
| A22 | LO-173 [DPM- 2011- 94] | 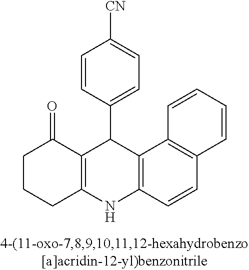 [SMILES: N#Cc1ccc(C2C3=C(CCCC3=O)Nc3ccc4ccccc4c32)cc1] |
| A23 | LO-128 | 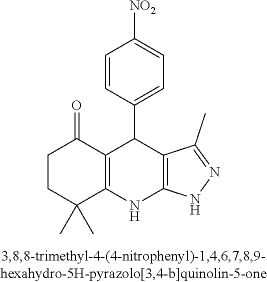 [SMILES: Cc1n[nH]c2c1C(c1ccc([N+](=O)[O-])cc1)C1=C(N2)C(C)(C)CCC1=O] |
| A24 | LO-148 | 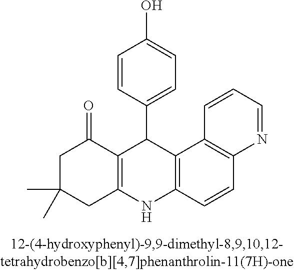 [SMILES: CC1(C)CC(=O)C2=C(C1)Nc1ccc3ncccc3c1C2c1ccc(O)cc1] |
| A25 | LO-38 | 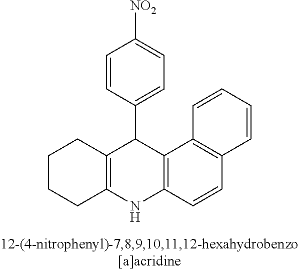 [SMILES: O=[N+]([O-])c1ccc(C2C3=C(CCCC3)Nc3ccc4ccccc4c32)cc1] |
| A26 | DPM- 2012- 23 | 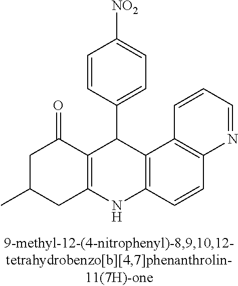 [SMILES: CC1CC(=O)C2=C(C1)Nc1ccc3ncccc3c1C2c1ccc([N+](=O)[O-])cc1] |
| A27 | DPM- 2012- 03 | 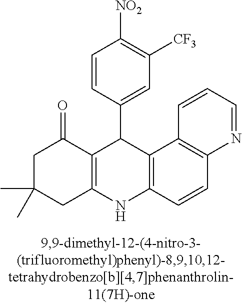 [SMILES: CC1(C)CC(=O)C2=C(C1)Nc1ccc3ncccc3c1C2c1ccc([N+](=O)[O-])c(C(F)(F)F)c1] |
| A28 | DPM- 2012- 02 | 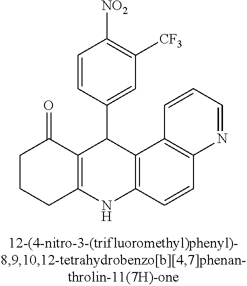 [SMILES: O=C1CCCC2=C1C(c1ccc([N+](=O)[O-])c(C(F)(F)F)c1)c1c(ccc3ncccc13)N2] |
| A29 | DPM- 2012- 01 | 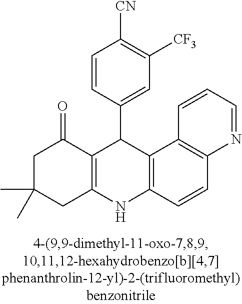 [SMILES: CC1(C)CC(=O)C2=C(C1)Nc1ccc3ncccc3c1C2c1ccc(C#N)c(C(F)(F)F)c1] |
| A30 | DPM- 2012-31 | 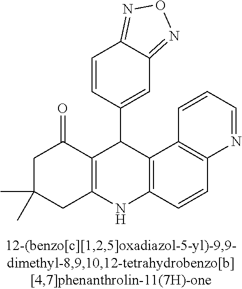 [SMILES: CC1(C)CC(=O)C2=C(C1)Nc1ccc3ncccc3c1C2c1ccc2nonc2c1] |
| A31 | DPM- 2012- 32 | 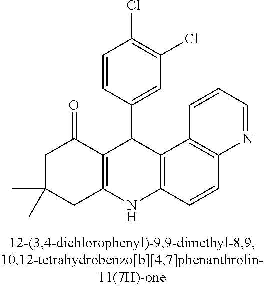 [SMILES: CC1(C)CC(=O)C2=C(C1)Nc1ccc3ncccc3c1C2c1ccc(Cl)c(Cl)c1] |
| A32 | DPM- 2012- 33 | 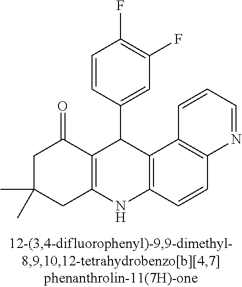 [SMILES: CC1(C)CC(=O)C2=C(C1)Nc1ccc3ncccc3c1C2c1ccc(F)c(F)c1] |
| A33 | DPM- 2012- 34 | 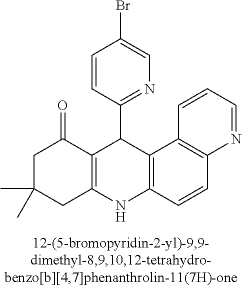 [SMILES: CC1(C)CC(=O)C2=C(C1)Nc1ccc3ncccc3c1C2c1ccc(Br)cn1] |
| A34 | DPM- 2012- 35 | 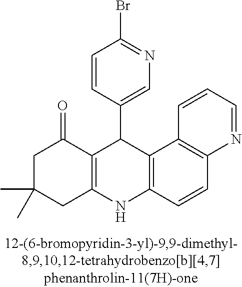 [SMILES: CC1(C)CC(=O)C2=C(C1)Nc1ccc3ncccc3c1C2c1ccc(Br)nc1] |
| A35 | DPM- 2012- 36 | 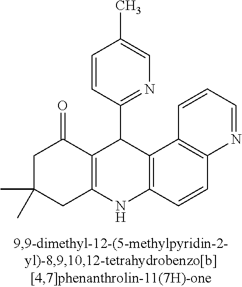 [SMILES: Cc1ccc(C2C3=C(CC(C)(C)CC3=O)Nc3ccc4ncccc4c32)nc1] |
| A36 | DPM- 2012- 37 | 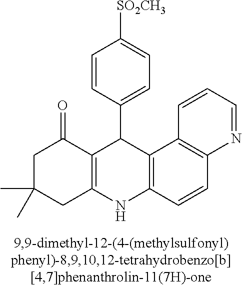 [SMILES: CC1(C)CC(=O)C2=C(C1)Nc1ccc3ncccc3c1C2c1ccc(S(C)(=O)=O)cc1] |
| A37 | DPM- 2012- 38 | 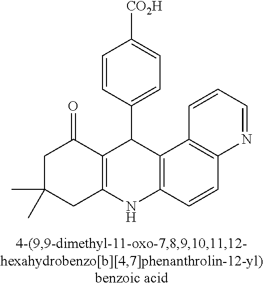 [SMILES: CC1(C)CC(=O)C2=C(C1)Nc1ccc3ncccc3c1C2c1ccc(C(=O)O)cc1] |
| A38 | DPM- 2012- 39 | 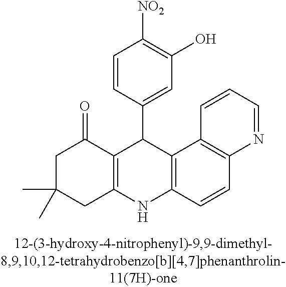 [SMILES: CC1(C)CC(=O)C2=C(C1)Nc1ccc3ncccc3c1C2c1ccc([N+](=O)[O-])c(O)c1] |
| A39 | DPM- 2012- 40 | 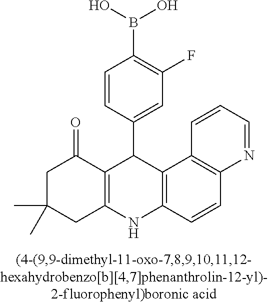 [SMILES: CC1(C)CC(=O)C2=C(C1)Nc1ccc3ncccc3c1C2c1ccc(B(O)O)c(F)c1] |
| A40 | DPM- 2012- 41 | 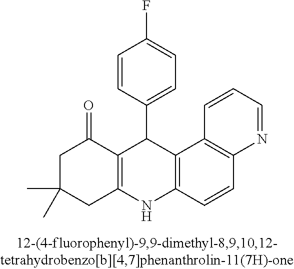 [SMILES: CC1(C)CC(=O)C2=C(C1)Nc1ccc3ncccc3c1C2c1ccc(F)cc1] |
| A41 | DPM- 2012- 42 | 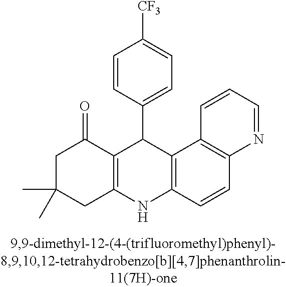 [SMILES: CC1(C)CC(=O)C2=C(C1)Nc1ccc3ncccc3c1C2c1ccc(C(F)(F)F)cc1] |
| A42 | DPM- 2012- 43 | 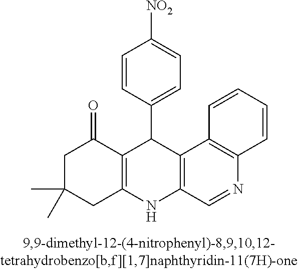 [SMILES: CC1(C)CC(=O)C2=C(C1)Nc1cnc3ccccc3c1C2c1ccc([N+](=O)[O-])cc1] |
| A43 | DPM- 2012- 44 | 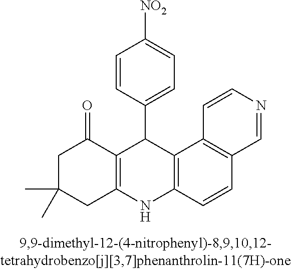 [SMILES: CC1(C)CC(=O)C2=C(C1)Nc1ccc3cnccc3c1C2c1ccc([N+](=O)[O-])cc1] |
| A44 | DPM- 2012- 45 | 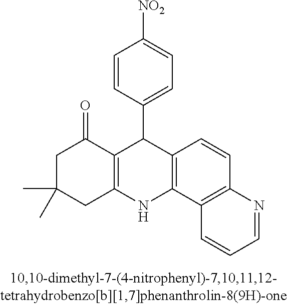 [SMILES: CC1(C)CC(=O)C2=C(C1)Nc1c(ccc3ncccc13)C2c1ccc([N+](=O)[O-])cc1] |
| A45 | DPM- 2012- 47 | 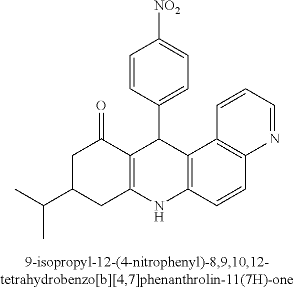 [SMILES: CC(C)C1CC(=O)C2=C(C1)Nc1ccc3ncccc3c1C2c1ccc([N+](=O)[O-])cc1] |
| A46 | DPM- 2012- 22 | 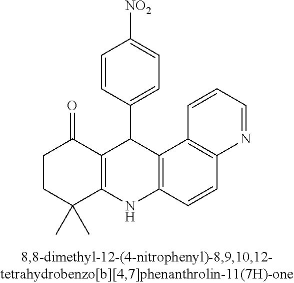 [SMILES: CC1(C)CCC(=O)C2=C1Nc1ccc3ncccc3c1C2c1ccc([N+](=O)[O-])cc1] |
| A47 | DPM- 2012- 46 | 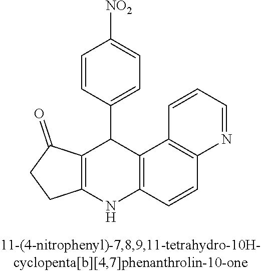 [SMILES: O=C1CCC2=C1C(c1ccc([N+](=O)[O-])cc1)c1c(ccc3ncccc13)N2] |
| A48 | DPM- 2012- 04 | 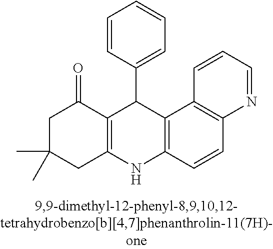 [SMILES: CC1(C)CC(=O)C2=C(C1)Nc1ccc3ncccc3c1C2c1ccccc1] |
| A49 | JJ- 2016- 034 | 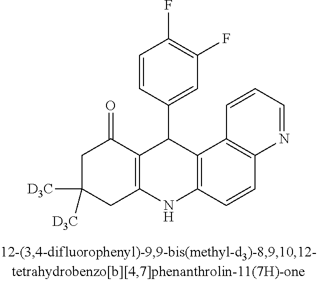 [SMILES: [2H]C([2H])([2H])C1(C([2H])([2H])[2H])CC(=O)C2=C(C1)Nc1ccc3ncccc3c1C2c1ccc(F)c(F)c1] |
| A50 | JJ- 2016- 042 | 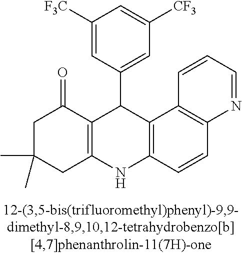 [SMILES: CC1(C)CC(=O)C2=C(C1)Nc1ccc3ncccc3c1C2c1cc(C(F)(F)F)cc(C(F)(F)F)c1] |
| A51 | JJ- 2016- 043 | 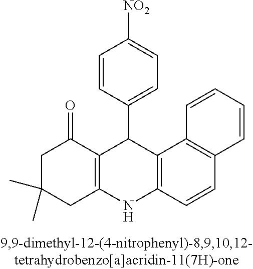 [SMILES: CC1(C)CC(=O)C2=C(C1)Nc1ccc3ccccc3c1C2c1ccc([N+](=O)[O-])cc1] |
| A52 | JJ- 2016- 045 | 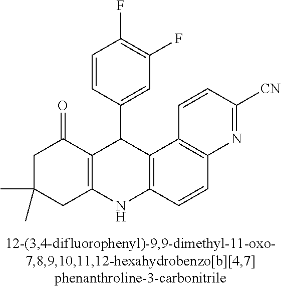 [SMILES: CC1(C)CC(=O)C2=C(C1)Nc1ccc3nc(C#N)ccc3c1C2c1ccc(F)c(F)c1] |
| A53 | JJ- 2016- 046 | 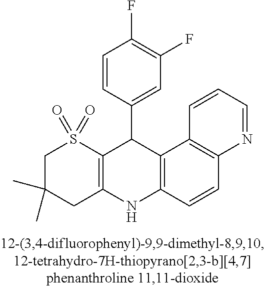 [SMILES: CC1(C)CC2=C(C(c3ccc(F)c(F)c3)c3c(ccc4ncccc34)N2)S(=O)(=O)C1] |
| A54 | JJ- 2016- 047 | 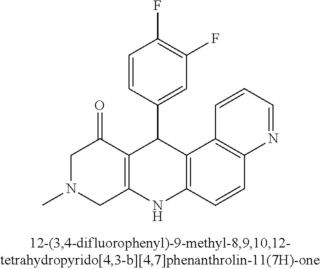 [SMILES: CN1CC(=O)C2=C(C1)Nc1ccc3ncccc3c1C2c1ccc(F)c(F)c1] |
| A55 | JJ- 2016- 050 | 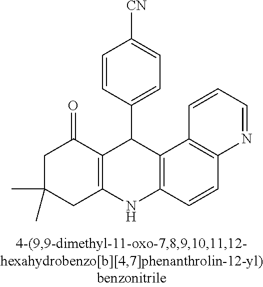 [SMILES: CC1(C)CC(=O)C2=C(C1)Nc1ccc3ncccc3c1C2c1ccc(C#N)cc1] |
| A56 | JJ- 2016- 051 | 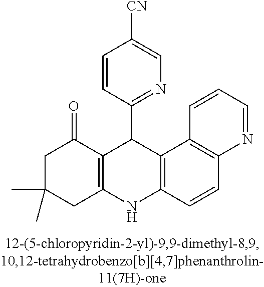 [SMILES: CC1(C)CC(=O)C2=C(C1)Nc1ccc3ncccc3c1C2c1ccc(C#N)cn1] |
| A57 | JJ- 2016- 052 | 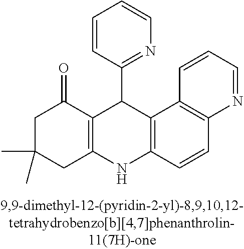 [SMILES: CC1(C)CC(=O)C2=C(C1)Nc1ccc3ncccc3c1C2c1ccccn1] |
| A58 | JJ- 2016- 053 | 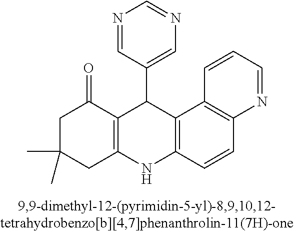 [SMILES: CC1(C)CC(=O)C2=C(C1)Nc1ccc3ncccc3c1C2c1cncnc1] |
| A59 | JJ- 2016- 054 | 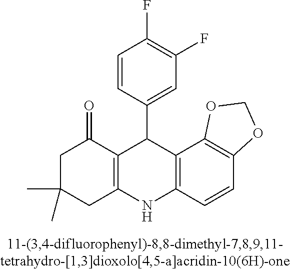 [SMILES: CC1(C)CC(=O)C2=C(C1)Nc1ccc3c(c1C2c1ccc(F)c(F)c1)OCO3] |
| A60 | JJ- 2016- 055 | 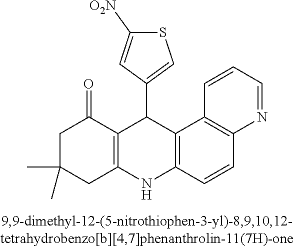 [SMILES: CC1(C)CC(=O)C2=C(C1)Nc1ccc3ncccc3c1C2c1csc([N+](=O)[O-])c1] |
| A61 | JJ- 2016- 056 | 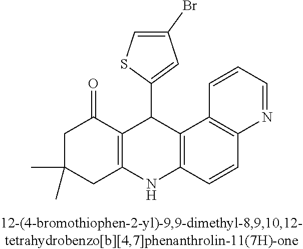 [SMILES: CC1(C)CC(=O)C2=C(C1)Nc1ccc3ncccc3c1C2c1cc(Br)cs1] |
| A62 | JJ- 2016- 061 | 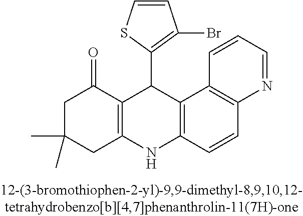 [SMILES: CC1(C)CC(=O)C2=C(C1)Nc1ccc3ncccc3c1C2c1sccc1Br] |
| A63 | JJ- 2016- 062 | 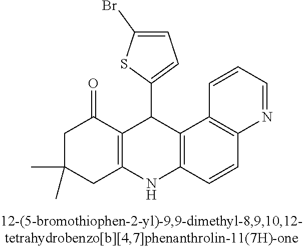 [SMILES: CC1(C)CC(=O)C2=C(C1)Nc1ccc3ncccc3c1C2c1ccc(Br)s1] |
| A64 | JJ- 2016- 063 | 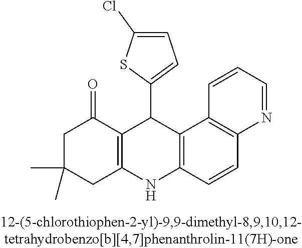 [SMILES: CC1(C)CC(=O)C2=C(C1)Nc1ccc3ncccc3c1C2c1ccc(Cl)s1] |
| A65 | JJ- 2016- 064 | 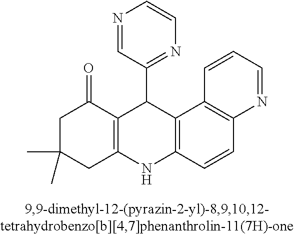 [SMILES: CC1(C)CC(=O)C2=C(C1)Nc1ccc3ncccc3c1C2c1cnccn1] |
| A66 | JJ- 2016- 065 | 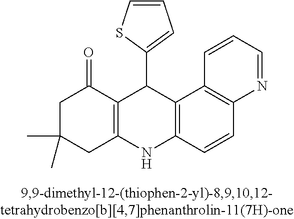 [SMILES: CC1(C)CC(=O)C2=C(C1)Nc1ccc3ncccc3c1C2c1cccs1] |
| A67 | JJ- 2016- 066 | 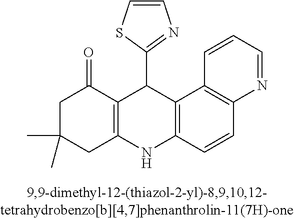 [SMILES: CC1(C)CC(=O)C2=C(C1)Nc1ccc3ncccc3c1C2c1nccs1] |
| A68 | JJ- 2016- 067 | 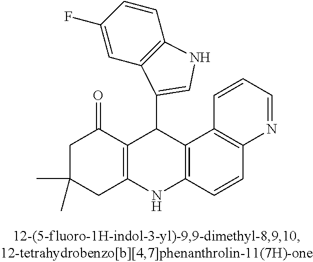 [SMILES: CC1(C)CC(=O)C2=C(C1)Nc1ccc3ncccc3c1C2c1c[nH]c2ccc(F)cc12] |
| A69 | JJ- 2016- 068 | 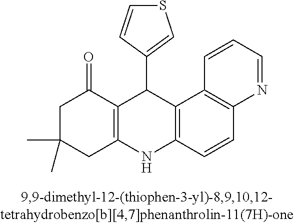 [SMILES: CC1(C)CC(=O)C2=C(C1)Nc1ccc3ncccc3c1C2c1ccsc1] |
| A70 | JJ- 2016- 072 | 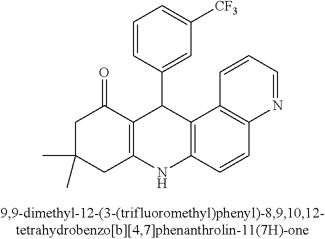 [SMILES: CC1(C)CC(=O)C2=C(C1)Nc1ccc3ncccc3c1C2c1cccc(C(F)(F)F)c1] |
| A71 | JJ- 2016- 073 | 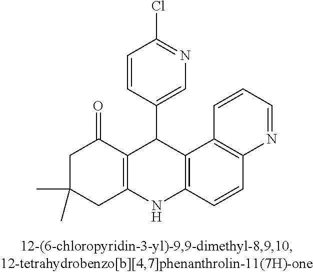 [SMILES: CC1(C)CC(=O)C2=C(C1)Nc1ccc3ncccc3c1C2c1ccc(Cl)nc1] |
| A72 | JJ- 2016- 074 | 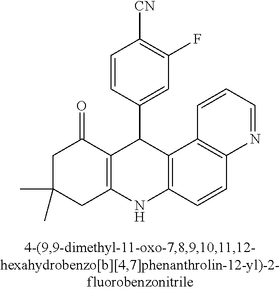 [SMILES: CC1(C)CC(=O)C2=C(C1)Nc1ccc3ncccc3c1C2c1ccc(C#N)c(F)c1] |
| A73 | JJ- 2016- 075 | 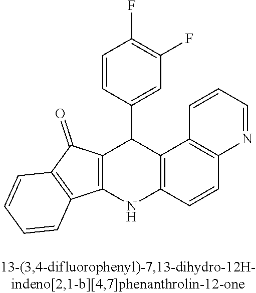 [SMILES: O=C1C2=C(Nc3ccc4ncccc4c3C2c2ccc(F)c(F)c2)c2ccccc21] |
| A74 | JJ- 2016- 076 | 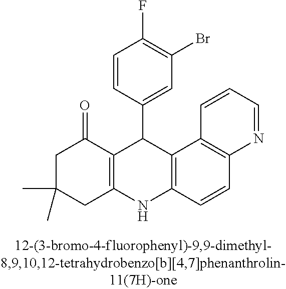 [SMILES: CC1(C)CC(=O)C2=C(C1)Nc1ccc3ncccc3c1C2c1ccc(F)c(Br)c1] |
| A75 | JJ- 2016- 077 | 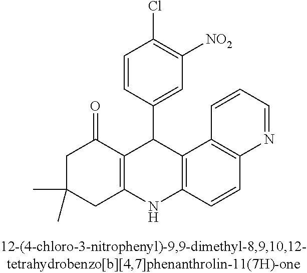 [SMILES: CC1(C)CC(=O)C2=C(C1)Nc1ccc3ncccc3c1C2c1ccc(Cl)c([N+](=O)[O-])c1] |
| A76 | JJ- 2016- 078 | 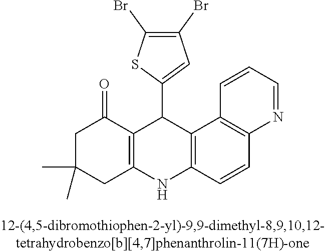 [SMILES: CC1(C)CC(=O)C2=C(C1)Nc1ccc3ncccc3c1C2c1cc(Br)c(Br)s1] |
| A77 | JJ- 2016- 079 | 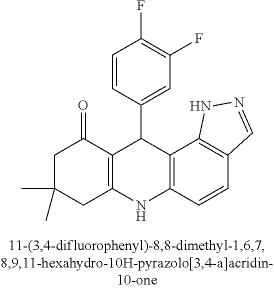 [SMILES: CC1(C)CC(=O)C2=C(C1)Nc1ccc3cn[nH]c3c1C2c1ccc(F)c(F)c1] |
| A78 | JJ- 2016- 080 | 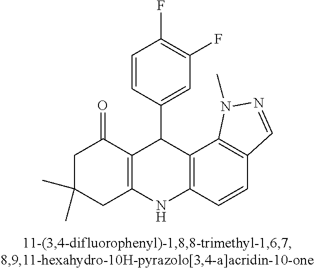 [SMILES: Cn1ncc2ccc3c(c21)C(c1ccc(F)c(F)c1)C1=C(CC(C)(C)CC1=O)N3] |
| A79 | JJ- 2016- 087 | 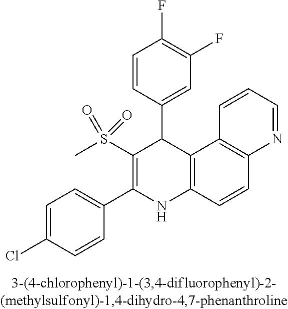 [SMILES: CS(=O)(=O)C1=C(c2ccc(Cl)cc2)Nc2ccc3ncccc3c2C1c1ccc(F)c(F)c1] |
| A80 | JJ- 2016- 093 | 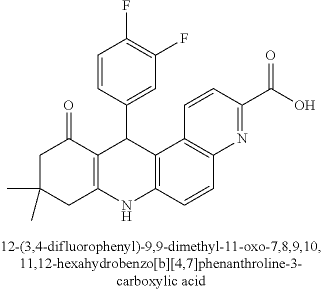 [SMILES: CC1(C)CC(=O)C2=C(C1)Nc1ccc3nc(C(=O)O)ccc3c1C2c1ccc(F)c(F)c1] |
| A81 | JJ- 2016- 094 | 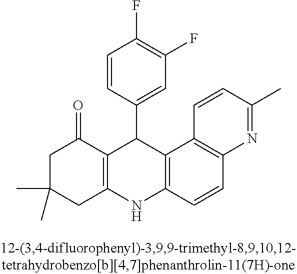 |
| A82 | JJ- 2016- 095 | 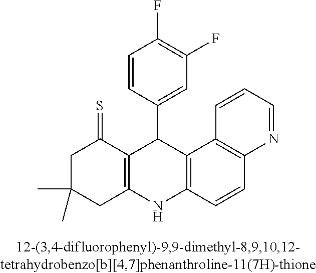 [SMILES: CC1(C)CC(=S)C2=C(C1)Nc1ccc3ncccc3c1C2c1ccc(F)c(F)c1] |
| A83 | JJ- 2016- 101 | 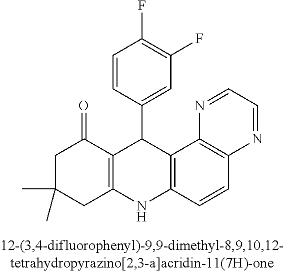 [SMILES: CC1(C)CC(=O)C2=C(C1)Nc1ccc3nccnc3c1C2c1ccc(F)c(F)c1] |
| A84 | JJ- 2016- 102 | 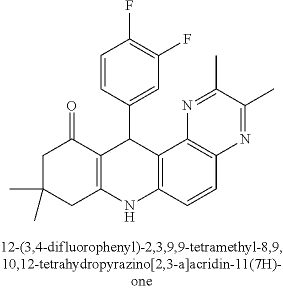 |
| A85 | JJ- 2016- 103 | 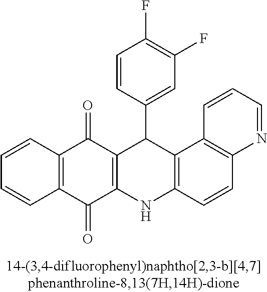 [SMILES: O=C1C2=C(C(=O)c3ccccc31)C(c1ccc(F)c(F)c1)c1c(ccc3ncccc13)N2] |
| A86 | JJ- 2016- 104 | 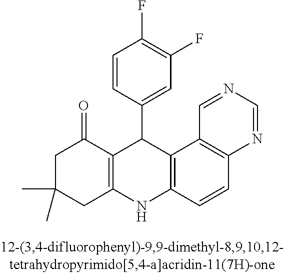 [SMILES: CC1(C)CC(=O)C2=C(C1)Nc1ccc3ncncc3c1C2c1ccc(F)c(F)c1] |
| A87 | JJ- 2016- 105 | 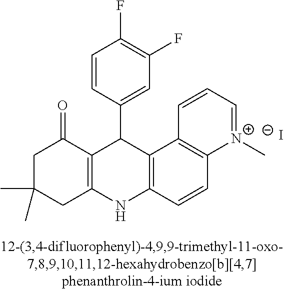 |
| A88 | JJ- 2016- 106 | 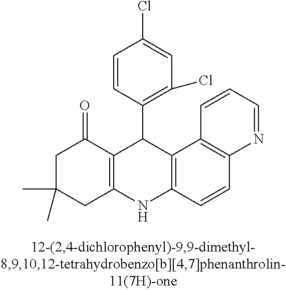 [SMILES: CC1(C)CC(=O)C2=C(C1)Nc1ccc3ncccc3c1C2c1ccc(Cl)cc1Cl] |
| A89 | JJ- 2016- 107 | 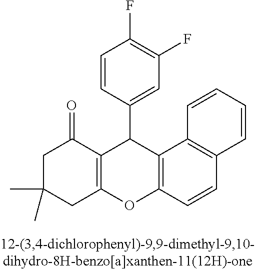 [SMILES: CC1(C)CC(=O)C2=C(C1)Oc1ccc3ccccc3c1C2c1ccc(F)c(F)c1] |
| A90 | JJ- 2016- 111 | 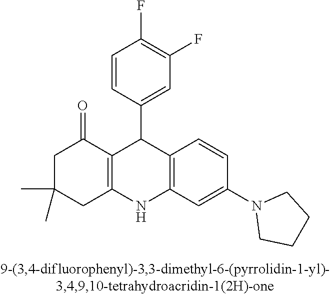 [SMILES: CC1(C)CC(=O)C2=C(C1)Nc1cc(N3CCCC3)ccc1C2c1ccc(F)c(F)c1] |
| A91 | JJ- 2016- 112 | 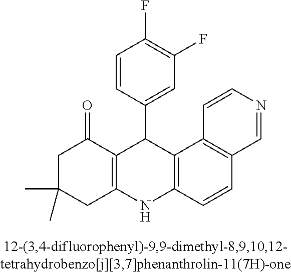 [SMILES: CC1(C)CC(=O)C2=C(C1)Nc1ccc3cnccc3c1C2c1ccc(F)c(F)c1] |
| A92 | JJ- 2016- 113 | 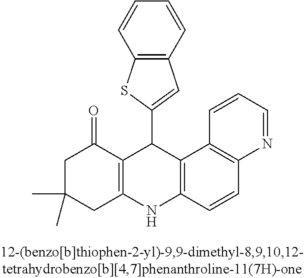 [SMILES: CC1(C)CC(=O)C2=C(C1)Nc1ccc3ncccc3c1C2c1cc2ccccc2s1] |
| A93 | JJ- 2016- 114 | 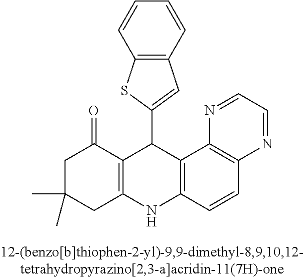 [SMILES: CC1(C)CC(=O)C2=C(C1)Nc1ccc3nccnc3c1C2c1cc2ccccc2s1] |
| A94 | JJ- 2016- 115 | 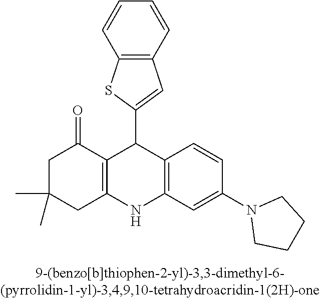 [SMILES: CC1(C)CC(=O)C2=C(C1)Nc1cc(N3CCCC3)ccc1C2c1cc2ccccc2s1] |
| A95 | JJ- 2016- 116 | 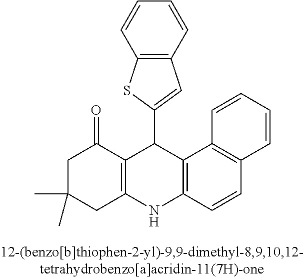 [SMILES: CC1(C)CC(=O)C2=C(C1)Nc1ccc3ccccc3c1C2c1cc2ccccc2s1] |
| A96 | JJ- 2016- 117 | 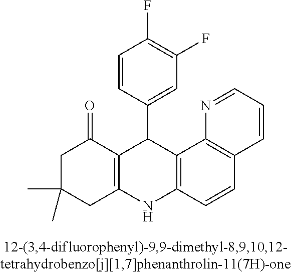 [SMILES: CC1(C)CC(=O)C2=C(C1)Nc1ccc3cccnc3c1C2c1ccc(F)c(F)c1] |
| A97 | JJ- 2016- 118 | 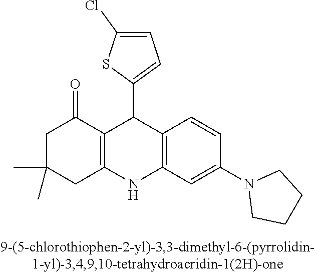 [SMILES: CC1(C)CC(=O)C2=C(C1)Nc1cc(N3CCCC3)ccc1C2c1ccc(Cl)s1] |
| A98 | JJ- 2016- 119 | 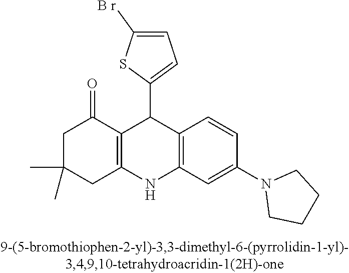 |
| A99 | JJ- 2016- 120 | 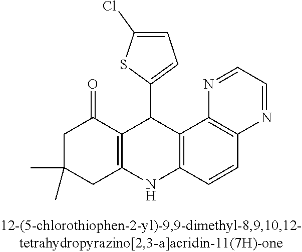 [SMILES: CC1(C)CC(=O)C2=C(C1)Nc1ccc3nccnc3c1C2c1ccc(Cl)s1] |
| A100 | JJ- 2016- 121 | 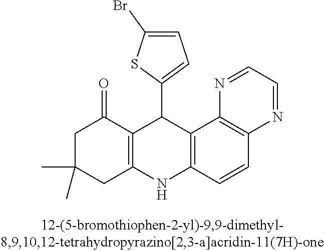 [SMILES: CC1(C)CC(=O)C2=C(C1)Nc1ccc3nccnc3c1C2c1ccc(Br)s1] |
| A101 | JJ- 2016- 122 | 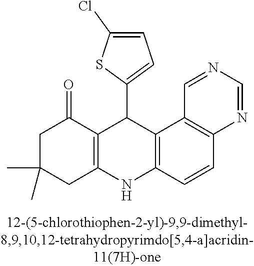 [SMILES: CC1(C)CC(=O)C2=C(C1)Nc1ccc3ncncc3c1C2c1ccc(Cl)s1] |
| A102 | JJ- 2016- 123 | 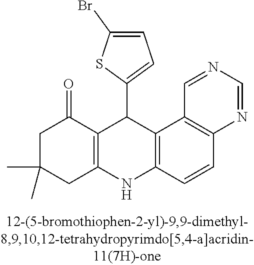 [SMILES: CC1(C)CC(=O)C2=C(C1)Nc1ccc3ncncc3c1C2c1ccc(Br)s1] |
| A103 | JJ- 2016- 124 | 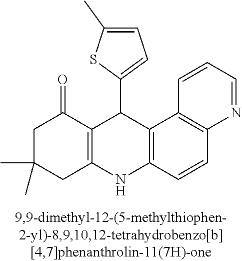 [SMILES: Cc1ccc(C2C3=C(CC(C)(C)CC3=O)Nc3ccc4ncccc4c32)s1] |
| A104 | JJ- 2016- 125 | 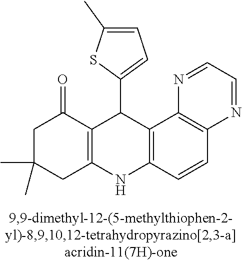 [SMILES: Cc1ccc(C2C3=C(CC(C)(C)CC3=O)Nc3ccc4nccnc4c32)s1] |
| A105 | JJ- 2016- 126 | 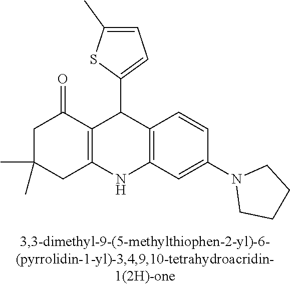 [SMILES: Cc1ccc(C2C3=C(CC(C)(C)CC3=O)Nc3cc(N4CCCC4)ccc32)s1] |
| A106 | JJ- 2016- 127 | 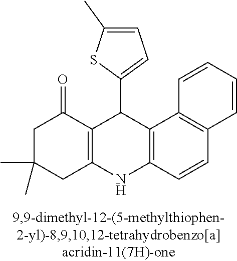 [SMILES: Cc1ccc(C2C3=C(CC(C)(C)CC3=O)Nc3ccc4ccccc4c32)s1] |
| A107 | JJ- 2016- 128 | 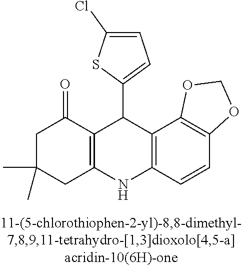 [SMILES: CC1(C)CC(=O)C2=C(C1)Nc1ccc3c(c1C2c1ccc(Cl)s1)OCO3] |
| A108 | JJ- 2016- 129 | 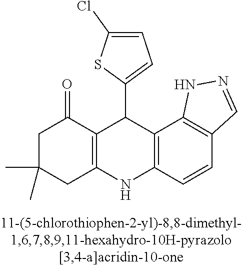 [SMILES: CC1(C)CC(=O)C2=C(C1)Nc1ccc3cn[nH]c3c1C2c1ccc(Cl)s1] |
| A109 | JJ- 2016- 130 | 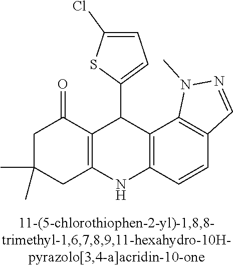 [SMILES: Cn1ncc2ccc3c(c21)C(c1ccc(Cl)s1)C1=C(CC(C)(C)CC1=O)N3] |
| A110 | JJ- 2016- 131 | 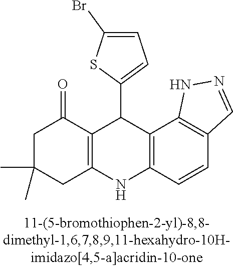 [SMILES: CC1(C)CC(=O)C2=C(C1)Nc1ccc3cn[nH]c3c1C2c1ccc(Br)s1] |
| A111 | JJ- 2016- 132 | 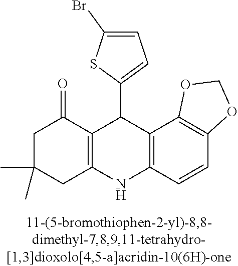 [SMILES: CC1(C)CC(=O)C2=C(C1)Nc1ccc3c(c1C2c1ccc(Br)s1)OCO3] |
| A112 | JJ- 2016- 133 | 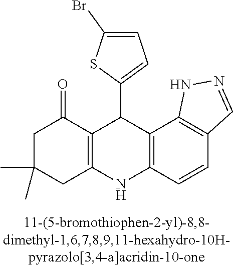 [SMILES: CC1(C)CC(=O)C2=C(C1)Nc1ccc3cn[nH]c3c1C2c1ccc(Br)s1] |
| A113 | JJ- 2016- 134 | 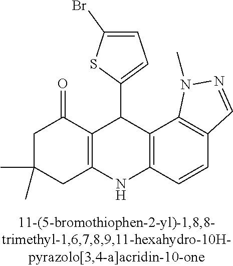 [SMILES: Cn1ncc2ccc3c(c21)C(c1ccc(Br)s1)C1=C(CC(C)(C)CC1=O)N3] |
| A114 | JJ- 2016- 135 | 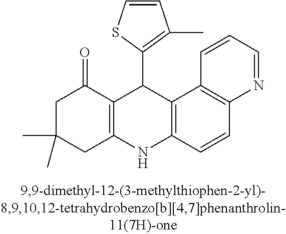 [SMILES: Cc1ccsc1C1C2=C(CC(C)(C)CC2=O)Nc2ccc3ncccc3c21] |
| A115 | JJ- 2016- 136 | 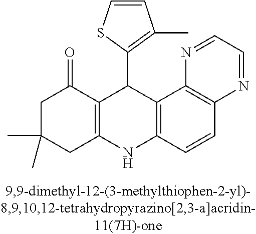 [SMILES: Cc1ccsc1C1C2=C(CC(C)(C)CC2=O)Nc2ccc3nccnc3c21] |
| A116 | JJ- 2016- 137 | 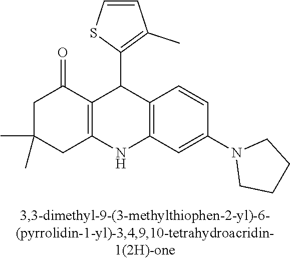 [SMILES: Cc1ccsc1C1C2=C(CC(C)(C)CC2=O)Nc2cc(N3CCCC3)ccc21] |
| A117 | JJ- 2016- 138 | 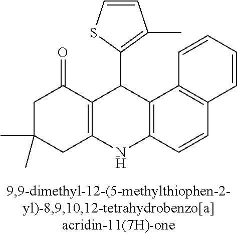 [SMILES: Cc1ccsc1C1C2=C(CC(C)(C)CC2=O)Nc2ccc3ccccc3c21] |
| A118 | JJ- 2016- 163 | 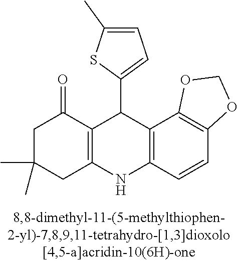 [SMILES: Cc1ccc(C2C3=C(CC(C)(C)CC3=O)Nc3ccc4c(c32)OCO4)s1] |
| A119 | JJ- 2016- 164 | 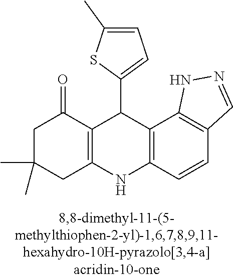 [SMILES: Cc1ccc(C2C3=C(CC(C)(C)CC3=O)Nc3ccc4cn[nH]c4c32)s1] |
| A120 | JJ- 2016- 165 | 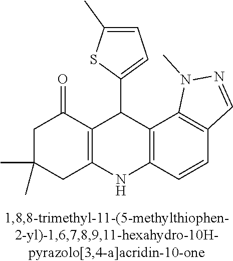 [SMILES: Cc1ccc(C2C3=C(CC(C)(C)CC3=O)Nc3ccc4cnn(C)c4c32)s1] |
| A121 | JJ- 2016- 166 | 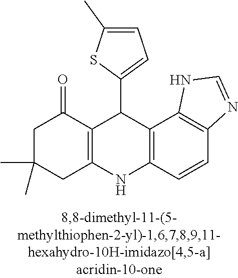 [SMILES: Cc1ccc(C2C3=C(CC(C)(C)CC3=O)Nc3ccc4nc[nH]c4c32)s1] |
| A122 | JJ- 2016- 167 | 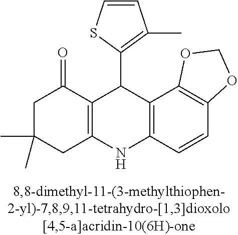 [SMILES: Cc1ccsc1C1C2=C(CC(C)(C)CC2=O)Nc2ccc3c(c21)OCO3] |
| A123 | JJ- 2016- 168 | 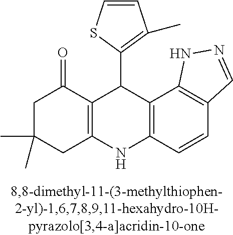 [SMILES: Cc1ccsc1C1C2=C(CC(C)(C)CC2=O)Nc2ccc3cn[nH]c3c21] |
| A124 | JJ- 2016- 169 | 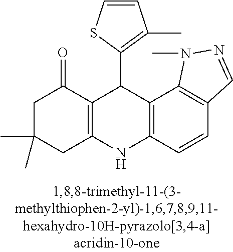 [SMILES: Cc1ccsc1C1C2=C(CC(C)(C)CC2=O)Nc2ccc3cnn(C)c3c21] |
| A125 | JJ- 2016- 170 | 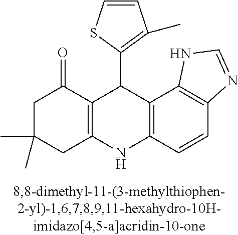 [SMILES: Cc1ccsc1C1C2=C(CC(C)(C)CC2=O)Nc2ccc3nc[nH]c3c21] |
| A126 | JJ- 2016- 171 | 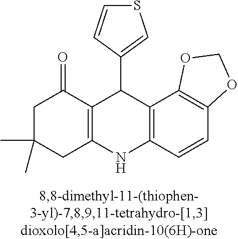 [SMILES: CC1(C)CC(=O)C2=C(C1)Nc1ccc3c(c1C2c1ccsc1)OCO3] |
| A127 | JJ- 2016- 172 | 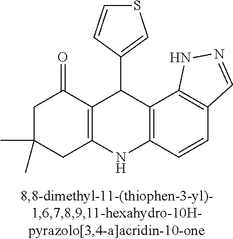 [SMILES: CC1(C)CC(=O)C2=C(C1)Nc1ccc3cn[nH]c3c1C2c1ccsc1] |
| A128 | JJ- 2016- 173 | 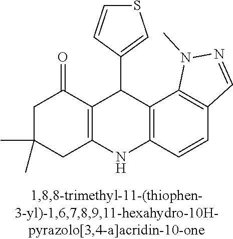 [SMILES: Cn1ncc2ccc3c(c21)C(c1ccsc1)C1=C(CC(C)(C)CC1=O)N3] |
| A129 | JJ- 2016- 174 | 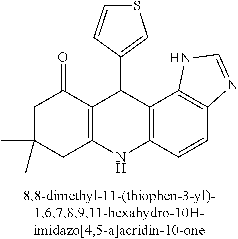 [SMILES: CC1(C)CC(=O)C2=C(C1)Nc1ccc3nc[nH]c3c1C2c1ccsc1] |
| A130 | JJ- 2016- 175 | 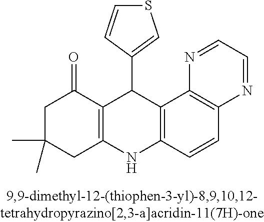 [SMILES: CC1(C)CC(=O)C2=C(C1)Nc1ccc3nccnc3c1C2c1ccsc1] |
| A131 | JJ- 2016- 176 | 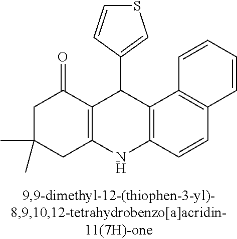 [SMILES: CC1(C)CC(=O)C2=C(C1)Nc1ccc3ccccc3c1C2c1ccsc1] |
| A132 | JJ- 2016- 177 | 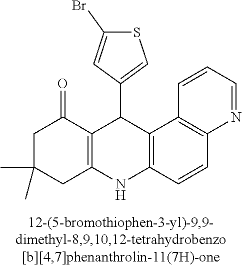 [SMILES: CC1(C)CC(=O)C2=C(C1)Nc1ccc3ncccc3c1C2c1csc(Br)c1] |
| A133 | JJ- 2016- 178 | 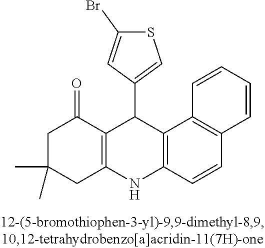 [SMILES: CC1(C)CC(=O)C2=C(C1)Nc1ccc3ccccc3c1C2c1csc(Br)c1] |
| A134 | JJ- 2016- 179 | 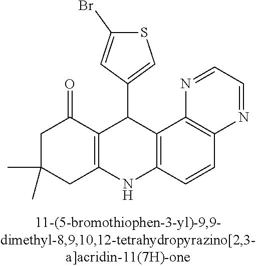 [SMILES: CC1(C)CC(=O)C2=C(C1)Nc1ccc3nccnc3c1C2c1csc(Br)c1] |
| A135 | JJ- 2016- 180 | 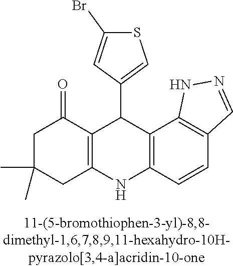 [SMILES: CC1(C)CC(=O)C2=C(C1)Nc1ccc3cn[nH]c3c1C2c1csc(Br)c1] |
| A136 | JJ- 2016- 181 | 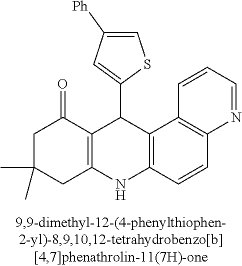 [SMILES: CC1(C)CC(=O)C2=C(C1)Nc1ccc3ncccc3c1C2c1cc(-c2ccccc2)cs1] |
| A137 | JJ- 2016- 182 | 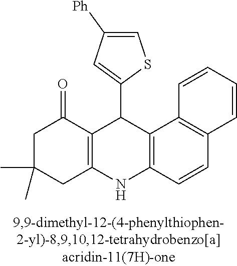 [SMILES: CC1(C)CC(=O)C2=C(C1)Nc1ccc3ccccc3c1C2c1cc(-c2ccccc2)cs1] |
| A138 | JJ- 2016- 183 | 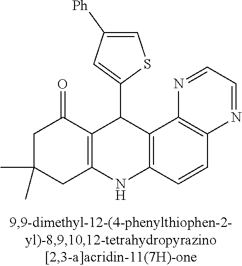 [SMILES: CC1(C)CC(=O)C2=C(C1)Nc1ccc3nccnc3c1C2c1cc(-c2ccccc2)cs1] |
| A139 | JJ- 2016- 184 | 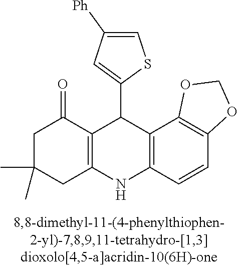 [SMILES: CC1(C)CC(=O)C2=C(C1)Nc1ccc3c(c1C2c1cc(-c2ccccc2)cs1)OCO3] |
| A140 | JJ- 2016- 185 | 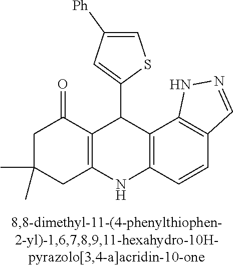 [SMILES: CC1(C)CC(=O)C2=C(C1)Nc1ccc3cn[nH]c3c1C2c1cc(-c2ccccc2)cs1] |
| A141 | JJ- 2016- 186 | 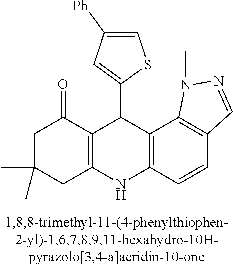 [SMILES: Cn1ncc2ccc3c(c21)C(c1cc(-c2ccccc2)cs1)C1=C(CC(C)(C)CC1=O)N3] |
| A142 | JJ- 2016- 187 | 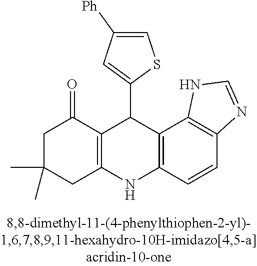 [SMILES: CC1(C)CC(=O)C2=C(C1)Nc1ccc3nc[nH]c3c1C2c1cc(-c2ccccc2)cs1] |
| A143 | JJ- 2016- 188 | 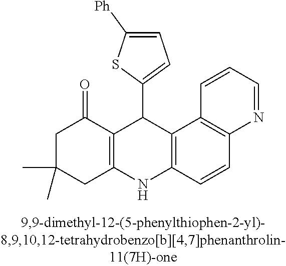 [SMILES: CC1(C)CC(=O)C2=C(C1)Nc1ccc3ncccc3c1C2c1ccc(-c2ccccc2)s1] |
| A144 | JJ- 2016- 189 | 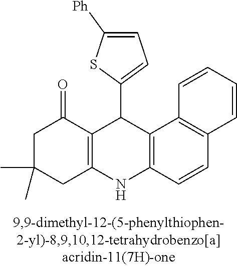 [SMILES: CC1(C)CC(=O)C2=C(C1)Nc1ccc3ccccc3c1C2c1ccc(-c2ccccc2)s1] |
| A145 | JJ- 2016- 190 | 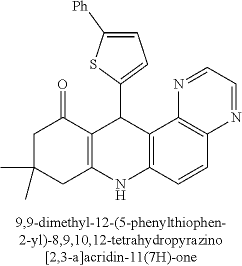 [SMILES: CC1(C)CC(=O)C2=C(C1)Nc1ccc3nccnc3c1C2c1ccc(-c2ccccc2)s1] |
| A146 | JJ- 2016- 191 | 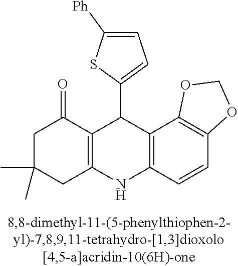 [SMILES: CC1(C)CC(=O)C2=C(C1)Nc1ccc3c(c1C2c1ccc(-c2ccccc2)s1)OCO3] |
| A147 | JJ- 2016- 192 | 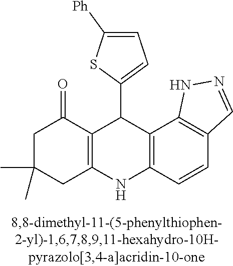 [SMILES: CC1(C)CC(=O)C2=C(C1)Nc1ccc3cn[nH]c3c1C2c1ccc(-c2ccccc2)s1] |
| A148 | JJ- 2016- 193 | 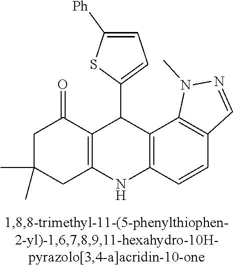 [SMILES: Cn1ncc2ccc3c(c21)C(c1ccc(-c2ccccc2)s1)C1=C(CC(C)(C)CC1=O)N3] |
| A149 | JJ- 2016- 194 | 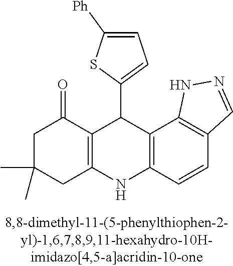 [SMILES: CC1(C)CC(=O)C2=C(C1)Nc1ccc3cn[nH]c3c1C2c1ccc(-c2ccccc2)s1] |
| A150 | JJ- 2016- 195 | 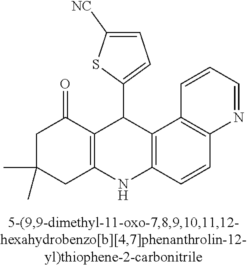 [SMILES: CC1(C)CC(=O)C2=C(C1)Nc1ccc3ncccc3c1C2c1ccc(C#N)s1] |
| A151 | JJ- 2016- 196 | 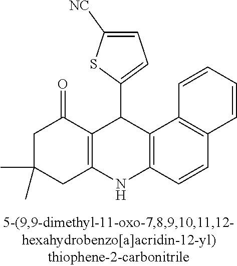 [SMILES: CC1(C)CC(=O)C2=C(C1)Nc1ccc3ccccc3c1C2c1ccc(C#N)s1] |
| A152 | JJ- 2016- 197 | 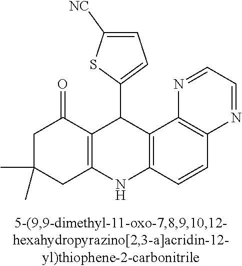 [SMILES: CC1(C)CC(=O)C2=C(C1)Nc1ccc3nccnc3c1C2c1ccc(C#N)s1] |
| A153 | JJ- 2016- 198 | 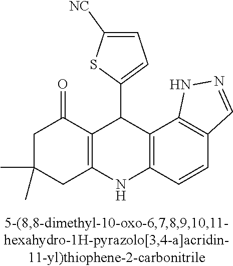 [SMILES: CC1(C)CC(=O)C2=C(C1)Nc1ccc3cn[nH]c3c1C2c1ccc(C#N)s1] |
| A154 | JJ- 2016- 199 | 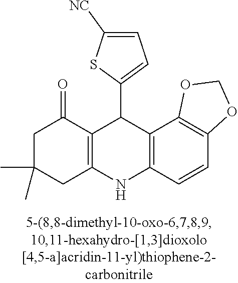 [SMILES: CC1(C)CC(=O)C2=C(C1)Nc1ccc3c(c1C2c1ccc(C#N)s1)OCO3] |
| A155 | JJ- 2016- 200 | 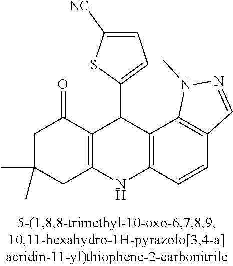 [SMILES: Cn1ncc2ccc3c(c21)C(c1ccc(C#N)s1)C1=C(CC(C)(C)CC1=O)N3] |
| A156 | JJ- 2016- 201 | 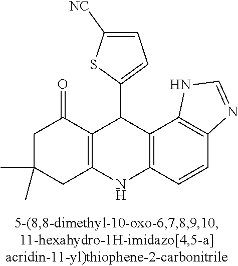 [SMILES: CC1(C)CC(=O)C2=C(C1)Nc1ccc3nc[nH]c3c1C2c1ccc(C#N)s1] |
| A157 | JJ- 2016- 202 | 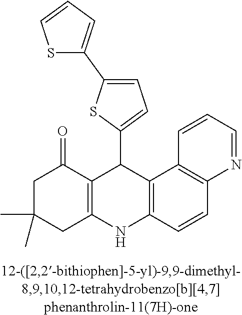 [SMILES: CC1(C)CC(=O)C2=C(C1)Nc1ccc3ncccc3c1C2c1ccc(-c2cccs2)s1] |
| A158 | JJ- 2016- 203 | 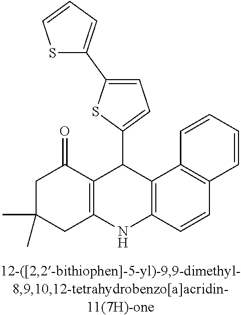 [SMILES: CC1(C)CC(=O)C2=C(C1)Nc1ccc3ccccc3c1C2c1ccc(-c2cccs2)s1] |
| A159 | JJ- 2016- 204 | 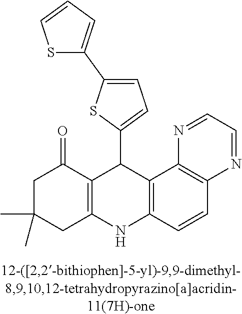 [SMILES: CC1(C)CC(=O)C2=C(C1)Nc1ccc3nccnc3c1C2c1ccc(-c2cccs2)s1] |
| A160 | JJ- 2016- 205 | 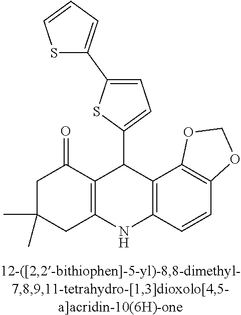 [SMILES: CC1(C)CC(=O)C2=C(C1)Nc1ccc3c(c1C2c1ccc(-c2cccs2)s1)OCO3] |
| A161 | JJ- 2016- 206 | 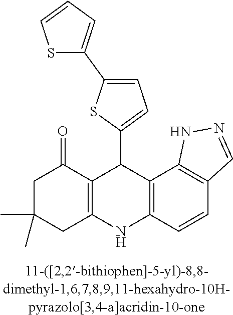 [SMILES: CC1(C)CC(=O)C2=C(C1)Nc1ccc3cn[nH]c3c1C2c1ccc(-c2cccs2)s1] |
| A162 | JJ- 2016- 207 | 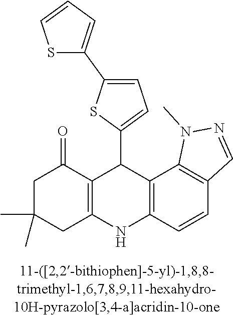 [SMILES: Cn1ncc2ccc3c(c21)C(c1ccc(-c2cccs2)s1)C1=C(CC(C)(C)CC1=O)N3] |
| A163 | JJ- 2016- 208 | 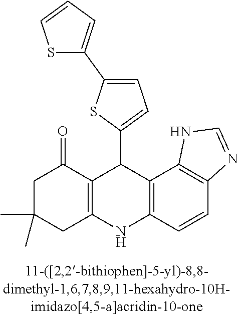 [SMILES: CC1(C)CC(=O)C2=C(C1)Nc1ccc3nc[nH]c3c1C2c1ccc(-c2cccs2)s1] |
| A164 | JJ- 2016- 209 | 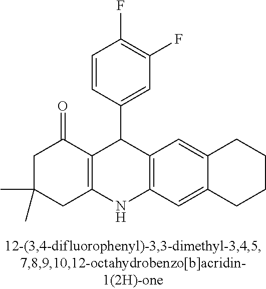 [SMILES: CC1(C)CC(=O)C2=C(C1)Nc1cc3c(cc1C2c1ccc(F)c(F)c1)CCCC3] |
| A165 | JJ- 2016- 210 | 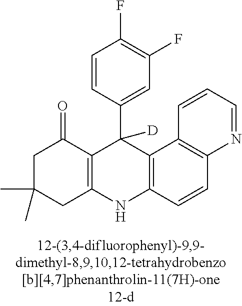 [SMILES: [2H]C1(c2ccc(F)c(F)c2)C2=C(CC(C)(C)CC2=O)Nc2ccc3ncccc3c21] |
| A166 | JJ- 2016- 211 | 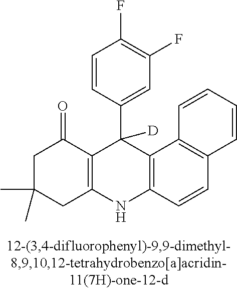 [SMILES: [2H]C1(c2ccc(F)c(F)c2)C2=C(CC(C)(C)CC2=O)Nc2ccc3ccccc3c21] |
| A167 | JJ- 2016- 212 | 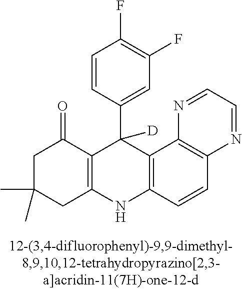 [SMILES: [2H]C1(c2ccc(F)c(F)c2)C2=C(CC(C)(C)CC2=O)Nc2ccc3nccnc3c21] |
| A168 | JJ- 2016- 213 | 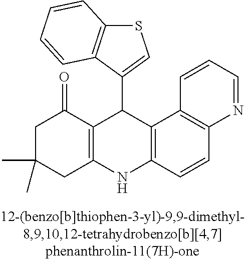 [SMILES: CC1(C)CC(=O)C2=C(C1)Nc1ccc3ncccc3c1C2c1csc2ccccc12] |
| A169 | JJ- 2016- 214 | 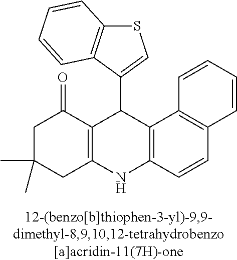 [SMILES: CC1(C)CC(=O)C2=C(C1)Nc1ccc3ccccc3c1C2c1csc2ccccc12] |
| A170 | JJ- 2016- 215 | 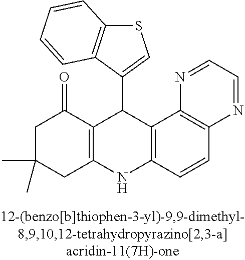 [SMILES: CC1(C)CC(=O)C2=C(C1)Nc1ccc3nccnc3c1C2c1csc2ccccc12] |
| A171 | JJ- 2016- 216 | 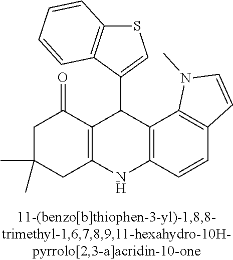 [SMILES: Cn1ccc2ccc3c(c21)C(c1csc2ccccc12)C1=C(CC(C)(C)CC1=O)N3] |
| A172 | JJ- 2016- 217 | 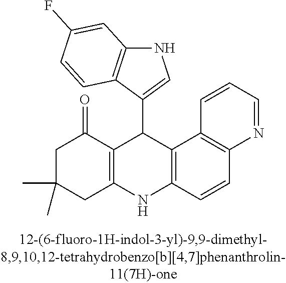 [SMILES: CC1(C)CC(=O)C2=C(C1)Nc1ccc3ncccc3c1C2c1c[nH]c2cc(F)ccc12] |
| A173 | JJ- 2016- 218 | 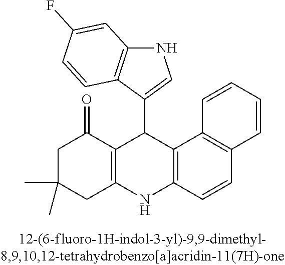 [SMILES: CC1(C)CC(=O)C2=C(C1)Nc1ccc3ccccc3c1C2c1c[nH]c2cc(F)ccc12] |
| A174 | JJ- 2016- 219 | 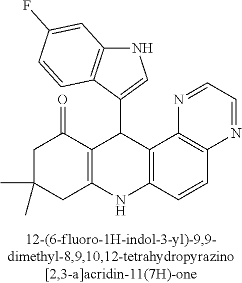 [SMILES: CC1(C)CC(=O)C2=C(C1)Nc1ccc3nccnc3c1C2c1c[nH]c2cc(F)ccc12] |
| A175 | JJ- 2016- 220 | 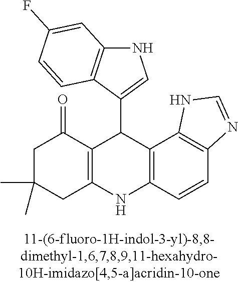 [SMILES: CC1(C)CC(=O)C2=C(C1)Nc1ccc3nc[nH]c3c1C2c1c[nH]c2cc(F)ccc12] |

### Biological Data

Reporter Gene Assay (IC50 determination, Table 2, B1-B3). CV1 cells were seeded into 96-well cell culture plates and transfected with Lipofectin. For AR transcriptional assays, the DNA mixture consisted of pcDNA-AR, MMTV-Luc (androgen responsive reporter gene), and Renilla-Luc (for assessing transfection efficiency and toxicity). Following overnight incubation, cells were induced with hormone (0.1 nM R1881 (synthetic AR agonist) plus increasing concentrations of test compound) for 24 hrs. Cells were lysed and luciferase activity was quantified using Dual Luciferase Reagent (DLR). IC50 determination was assessed using GraphPad Prism software.

Real-Time PCR (CRPC model of AR overexpressing prostate cancer cells, Table 2, B4-B7). LNCaP-AR (AR overexpressing cells) cells were seeded in 12-well plates in RPMI 1640 (8% charcoal-stripped fetal calf serum (CFS)). After 48 hrs, cells were treated with ligand for 18 hrs (0.1 nM R1881 plus 20 μM test compound) and total RNA was isolated. AR target gene transcription (PSA, NKX3.1, FKBP5) was assessed by realtime PCR.

In Cell Western (AR degradation Assay, Table 2, B10). LNCaP cells were plated in 96-well clear bottom black plates (20K cells/well) in RPMI supplemented with 8% CFS. Following 48 hr incubation, cells were treated with hormone (20 μM test compound) for 18 hrs. Cells were fixed with formaldehyde (3.7%) and permeabilized using PBS (0.1% TRITON X-100). Cells were incubated with anti-AR antibody (N20, 1:2000), washed with PBS (0.1% Tween), and stained with 2^{nd} antibody (Biotium CF770 goat anti-rabbit, 1:2000). AR protein expression was assessed using the LI-COR Odyssey imaging system. DRAQ5 (DNA stain, 1:10,000, Thermo Scientific) was used to normalize AR protein expression.

TABLE 2
Screen Results

| Cmpd | B1 | B2 | B3 | B4 | B5 | B6 | B7 | B8 | B9 |
|---|---|---|---|---|---|---|---|---|---|
| A1 | 4.4 | 32 | 87 | 1 | 2 | 1 | 2 | 2 | Degrader |
| A2 | 0.2 | 0 | 51 | | | | 1 | 5 | |
| A3 | 1.6 | 0 | 24 | 1 | 1 | 1 | 1 | | |
| A4 | 2.6 | 0 | 51 | 1 | 1 | 1 | 1 | | Degrader |
| A5 | 3.3 | 24 | 68 | | | | | | |
| A6 | 3.8 | 0 | 0 | 1 | 1 | 1 | 1 | | |
| A7 | 3.8 | 75 | 91 | 1 | 2 | 1 | 1 | | |
| A8 | 5.3 | 0 | 0 | 2 | 1 | 1 | 1 | | |
| A9 | 5.5 | 0 | 0 | 1 | 1 | 1 | 1 | | |
| A10 | 5.7 | 17 | 88 | 2 | 2 | 1 | 1 | | Degrader |
| A11 | 8 | 46 | 62 | | | | | | |
| A12 | 8.6 | 0 | 94 | 1 | 2 | 1 | 3 | | |
| A13 | 12 | 0 | 78 | | | | | | |
| A14 | >10 | 0 | 0 | | | | | | |
| A15 | >10 | 0 | 0 | | | | | | |
| A16 | >10 | 0 | 0 | | | | | | |
| A17 | >10 | 0 | 36 | | | | | | |
| A18 | >10 | 0 | 22 | | | 1 | 2 | | |
| A19 | >10 | 0 | 87 | | | | | | |
| A20 | >10 | 0 | 0 | | | | | | |
| A21 | >10 | 0 | 28 | | | | | | |
| A22 | >10 | 0 | 66 | | | 3 | 3 | | |
| A23 | >10 | 0 | 0 | | | | | | |
| A24 | >10 | 0 | 35 | | | | | | |
| A25 | >10 | 23 | 42 | | | | | | |
| A26 | 0.43 | | | | | | | | |
| A27 | −7 | | | | | | | | |
| A28 | −8 | | | | | | | | |
| A29 | −7 | | | | | | | | |
| A30 | 1.8 | | | | | | | | |
| A31 | 2.2 | | | | | | | | |
| A32 | 0.4 | | | | | | | | |
| A33 | 3.4 | | | | | | | | |
| A34 | 1.9 | | | | | | | | |
| A35 | 58 | | | | | | | | |
| A36 | Inactive | | | | | | | | |
| A37 | 38 | | | | | | | | |
| A38 | 3.3 | | | | | | | | |
| A39 | 52 | | | | | | | | |
| A40 | 1.7 | | | | | | | | |
| A41 | 1.9 | | | | | | | | |
| A42 | — | | | | | | | | |
| A43 | 9.1 | | | | | | | | |
| A44 | Inactive | | | | | | | | |
| A45 | 0.3 | | | | | | | | |
| A46 | −2 | | | | | | | | |
| A47 | 9.9 | | | | | | | | |
| A48 | −5 | | | | | | | | |
| A49 | Not | | | | | | | | |
| | tested | | | | | | | | |
| A50 | 6.0 | | | | | | | | Degrader |
| A51 | 1.2 | | | | | | | | Degrader |
| A52 | >10 | | | | | | | | |
| A53 | >10 | | | | | | | | |
| A54 | >10 | | | | | | | | |
| A55 | >10 | | | | | | | | Degrader |
| A56 | 1.2 | | | | | | | | |
| A57 | >10 | | | | | | | | Degrader |
| A58 | >10 | | | | | | | | |
| A59 | >10 | | | | | | | | Degrader |
| A60 | 1.7 | | | | | | | | Degrader |
| A61 | 1.8 | | | | | | | | Degrader |
| A62 | >10 | | | | | | | | Degrader |
| A63 | 1.9 | | | | | | | | Degrader |
| A64 | 1.9 | | | | | | | | Degrader |
| A65 | >10 | | | | | | | | |
| A66 | 4.1 | | | | | | | | |
| A67 | >10 | | | | | | | | Degrader |
| A68 | >10 | | | | | | | | Degrader |
| A69 | 6.1 | | | | | | | | Degrader |
| A70 | >10 | | | | | | | | Degrader |
| A71 | 6.9 | | | | | | | | Degrader |
| A72 | 7.7 | | | | | | | | Degrader |
| A73 | Not | | | | | | | | |
| | tested | | | | | | | | |
| A74 | 8.6 | | | | | | | | Degrader |
| A75 | >10 | | | | | | | | Degrader |
| A76 | 2.2 | | | | | | | | Degrader |
| A77 | >10 | | | | | | | | |
| A78 | >10 | | | | | | | | Degrader |
| A79 | Not | | | | | | | | |
| | tested | | | | | | | | |
| A80 | >10 | | | | | | | | |
| A81 | >10 | | | | | | | | |
| A82 | 9.1 | | | | | | | | |
| A83 | 7.4 | | | | | | | | Degrader |
| A84 | 55 | | | | | | | | Degrader |
| A85 | >10 | | | | | | | | Degrader |
| A86 | 1.8 | | | | | | | | |
| A87 | 1.3 | | | | | | | | |
| A88 | 1.1 | | | | | | | | Degrader |
| A89 | 0.4 | | | | | | | | |
| A90 | >10 | | | | | | | | Degrader |
| A91 | 1.4 | | | | | | | | Degrader |
| A92 | 0.9 | | | | | | | | Degrader |
| A93 | 1.2 | | | | | | | | Degrader |
| A94 | >10 | | | | | | | | |
| A95 | 0.5 | | | | | | | | |
| A96 | 0.8 | | | | | | | | Degrader |
| A97 | >10 | | | | | | | | Degrader |
| A98 | >10 | | | | | | | | |
| A99 | 0.6 | | | | | | | | |
| A100 | 0.5 | | | | | | | | Degrader |
| A101 | 0.8 | | | | | | | | Degrader |
| A102 | 0.4 | | | | | | | | Degrader |
| A103 | 0.3 | | | | | | | | Degrader |
| A104 | 2.0 | | | | | | | | |
| A105 | >10 | | | | | | | | |
| A106 | 0.8 | | | | | | | | |
| A107 | 0.9 | | | | | | | | Degrader |
| A108 | 1.2 | | | | | | | | |
| A109 | 4.7 | | | | | | | | Degrader |
| A110 | >10 | | | | | | | | Degrader |
| A111 | 1.4 | | | | | | | | Degrader |
| A112 | 3.2 | | | | | | | | |
| A113 | 5.1 | | | | | | | | Degrader |
| A114 | 1.1 | | | | | | | | Degrader |
| A115 | >10 | | | | | | | | |
| A116 | >10 | | | | | | | | Degrader |
| A117 | 2.3 | | | | | | | | Degrader |

In Table 2, B1=AR transcriptional IC₅₀ (μM) in CV1 cells; B2=% Toxicity of test compound (20 μM); B3=% Toxicity of test compound (60 μM); B4=AR transcriptional IC₅₀ in CV1 cells; B4=Relative agonist activity (no R1881) of test compounds (20 μM) in AR overexpressing LNCaP cells (NKX3.1 AR target gene); B5=Relative antagonist activity (plus 0.1 nM R1881) of test compounds (20 μM) in AR overexpressing LNCaP cells (NKX3.1 AR target gene); B6 Relative agonist activity (no R1881) of test compounds (20 μM) in AR overexpressing LNCaP cells (PSA AR target gene) B7=Relative antagonist activity (plus 0.1 nM R1881) of test compounds (20 μM) in AR overexpressing LNCaP cells (PSA AR target gene); B9=AR degradation in LNCaP cells (In-Cell Western).

For B4-B7, scoring system is based on the relative AR target gene agonist/antagonist activity of test compounds when compared to benchmark compounds enzalutamide or bicalutamide. Enzalutamide does not possess agonist activity in AR overexpressing LNCaP cells (model of CRPC) while bicalutamide does possess agonist activity. The lack of agonist activity is desirable for therapies intended to treat CRPC. For B4 and B6, a score of 1 indicates less agonism (highly desired activity) than benchmark compound enzalutamide, a score of 2 indicates equal agonism to enzalutamide (desirable), a score of 3 indicates agonism between enzalutamide and bicalutamide (less desirable activity), a score of 4 indicates equal agonism to bicalutamide (liability), and a score of 5 indicates higher agonism than bicalutamide (liability). For B5 and B7 a score of 1 indicates higher antagonist efficacy (highly desired activity) than benchmark compound enzalutamide, a score of 2 indicates equal antagonist efficacy to enzalutamide (desirable), a score of 3 indicates antagonist efficacy between enzalutamide and bicalutamide (less desirable activity), a score of 4 indicates antagonist efficacy equal to bicalutamide (liability), and a score of 5 indicates antagonist efficacy worse than bicalutamide (liability) For B9, Compounds that degrade AR (greater than 50% degradation at 10 μM) are indicated.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A compound of formula (I), or a pharmaceutically acceptable salt thereof, 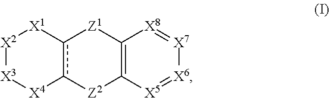
wherein
X¹ is C(R^{1a}R^{1b}), O, S, C(O), C(S), S(O), or S(O)₂, or N—R^{1c};
X² is a bond, C(R^{2a}R^{2b}), or N—R²;
X³ is a bond, C(R^{3a}R^{3b}), or N—R^{3c};
X⁴ is a bond, C(R^{4a}R^{4b}), C(R^{4a}R^{4b})—C(R^{4a}R^{4b}), C(O), C(S), S(O), S(O)₂, or N—R^{4c};
provided that no more than two of X²-X⁴ are simultaneously a bond;
R^{1a}, R^{1b}, R^{2a}, R^{2b}, R^{3a}, R^{3b}, R^{4a}, and R^{4b} are each independently selected from the group consisting of hydrogen, deuterium, halogen, alkyl, alkenyl, alkynyl, heteroalkyl, aryl, heteroaryl, cycloalkyl, heterocycloalkyl, and —SO₂-alkyl;
R^{1c}, R^{2c}, R^{3c}, and R^{4c} are each independently selected from the group consisting of hydrogen, deuterium, and alkyl;
optionally R^{1a} and R^{1b}, R^{2a} and R^{2b}, R^{3a} and R^{3b}, R^{4a} and R^{4b}, R^{1a} and R^{2a}, R^{2a} and R^{3a}, R^{3a} and R^{4a}, R^{1c} and R^{2a}, R^{1c} and R^{2c}, R^{1a} and R^{2c}, R^{2c} and R^{3a}, R^{2c} and R^{3c}, R^{2a} and R^{3c}, R^{3c} and R^{4a}, R^{3c} and R^{4c}, or R^{3a} and R^{4c} together with the carbon atoms to which they are attached form an aryl, heteroaryl, cycloalkyl, cycloalkenyl, or heterocycloalkyl;
X⁵ is C(R⁵), N, or a bond;
X⁶ is C(R⁶) or N;
X⁷ is C(R⁷) or N;
X⁸ is C(R⁸) or N;
Z¹ is C(R⁹R^{9′});
Z² is N—R¹⁰, C(R^{10′}R^{10″}), O, or S;
R⁵, R⁶, R⁷, R⁸, R⁹, R^{10′}, and R^{10″} are each independently selected from the group consisting of hydrogen, deuterium, halogen, alkyl, alkenyl, alkynyl, heteroalkyl, aryl, heteroaryl, cycloalkyl, heterocycloalkyl, cyano, nitro, hydroxy, alkoxy, amino, alkylamino, and dialkylamino; R^{9′} is hydrogen or deuterium; optionally R⁵ and R⁶, R⁶ and R⁷, or R⁷ and R⁸ together with the carbon atoms to which they are attached form an aryl, heteroaryl, cycloalkyl, cycloalkenyl, or heterocycloalkyl; and
R¹⁰ is selected from the group consisting of hydrogen, deuterium, and alkyl;
wherein said alkyl, alkenyl, alkynyl, heteroalkyl, alkoxy, aryl, heteroaryl, cycloalkyl, cycloalkenyl, and heterocycloalkyl, at each occurrence, whether alone or part of another group, are independently substituted with 0, 1, 2, 3, 4, or 5 substituents independently selected from the group consisting of deuterium, halogen, oxo (═O), ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycloalkyl, cycloalkylalkyl, cycloalkenylalkyl, heteroarylalkyl, arylalkyl, heterocycloalkylalkyl, hydroxy, hydroxyalkyl, alkoxy, alkylthio, alkoxyalkyl, alkylene, aryloxy, arylthio, phenoxy, benzyloxy, amino, alkylamino, dialkylamino, acylamino, aminoalkyl, arylamino, diarylamino, sulfonylamino, sulfinylamino, sulfonyl, alkylsulfonyl, arylsulfonyl, aminosulfonyl, sulfinyl, —COOH, ketone, alkoxycarbonyl, aryloxycarbonyl, amide, carbamate, acyl, boronic acid, and boronic ester.

2. The compound of claim 1, having formula (I′), 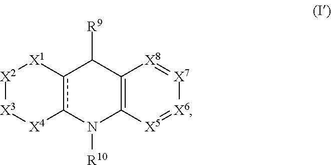
wherein
X¹ is C(R^{1a}R^{1b}), C(O), S(O), or S(O)₂;
X² is a bond or C(R^{2a}R^{2b});
X³ is C(R^{3a}R^{3b});
X⁴ is C(R^{4a}R^{4b}), C(O), S(O), or S(O)₂;
R^{1a}, R^{1b}, R^{2a}, R^{2b}, R^{3a}, R^{3b}, R^{4a}, and R^{4b} are each independently selected from the group consisting of hydrogen, halogen, alkyl, alkenyl, alkynyl, aryl, heteroaryl, cycloalkyl, heterocycloalkyl, and —SO₂-alkyl;
X⁵ is C(R⁵) or N;
X⁶ is C(R⁶) or N;
X⁷ is C(R⁷) or N;
X⁸ is C(R⁸) or N;
R⁵, R⁶, R⁷, and R⁸ are each independently selected from the group consisting of hydrogen, halogen, alkyl, alkenyl, alkynyl, aryl, heteroaryl, cycloalkyl, heterocycloalkyl, cyano, nitro, hydroxy, alkoxy, amino, alkylamino, and dialkylamino; optionally R⁵ and R⁶, R⁶ and R⁷, or R⁷ and R⁸ together with the carbon atoms to which they are attached form a 5- or 6-membered aryl, heteroaryl, cycloalkyl, cycloalkenyl, or heterocycloalkyl;
R⁹ is aryl, heteroaryl, cycloalkyl, or heterocycloalkyl; and
R¹⁰ is hydrogen or alkyl;
wherein said alkyl, alkenyl, alkynyl, alkoxy, aryl, heteroaryl, cycloalkyl, and heterocycloalkyl, at each occurrence, whether alone or part of another group, are independently substituted with 0, 1, 2, 3, 4, or 5 substituents independently selected from the group consisting of halogen, oxo (═O), ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycloalkyl, cycloalkylalkyl, cycloalkenylalkyl, heteroarylalkyl, arylalkyl, heterocycloalkylalkyl, hydroxy, hydroxyalkyl, alkoxy, alkylthio, alkoxyalkyl, alkylene, aryloxy, arylthio, phenoxy, benzyloxy, amino, alkylamino, dialkylamino, acylamino, aminoalkyl, arylamino, diarylamino, sulfonylamino, sulfinylamino, sulfonyl, alkylsulfonyl, arylsulfonyl, aminosulfonyl, sulfinyl, —COOH, ketone, alkoxycarbonyl, aryloxycarbonyl, amide, carbamate, acyl, boronic acid, and boronic ester.

3. The compound of claim 1 or claim 2, wherein X¹ is C(O).

4. The compound of any one of claim 1-3, wherein
X² is C(R^{2a}R^{2b});
R^{2a} is hydrogen; and
R^{2b} is hydrogen.

5. The compound of any one of claims 1-4, wherein
X³ is C(R^{3a}R^{3b});
R^{3a} is hydrogen, C₁-C₄-alkyl, or C₁-C₄-haloalkyl; and
R^{3b} is hydrogen, C₁-C₄-alkyl, or C₁-C₄-haloalkyl.

6. The compound of any one of claims 1-5, wherein
X³ is C(R^{3a}R^{3b});
R^{3a} is methyl; and
R^{3b} is methyl.

7. The compound of any one of claims 1-6, wherein
X⁴ is C(R^{4a}R^{4b});
R^{4a} is hydrogen; and
R^{4b} is hydrogen.

8. The compound of any one of claims 1-7, wherein
X⁵ is C(R⁵);
X⁶ is C(R⁶);
X⁷ is C(R⁷); and
X⁸ is C(R⁸).

9. The compound of any one of claims 1-8, wherein
X⁵ is C(R⁵);
X⁶ is C(R⁶);
X⁷ is C(R⁷);
X⁸ is C(R⁸); and
one of R⁵ and R⁶, or R⁷ and R⁸, together with the carbon atoms to which they are attached form an aryl, heteroaryl, cycloalkyl, cycloalkenyl, or heterocycloalkyl.

10. The compound of any one of claims 1-9, wherein
X⁵ is CH;
X⁶ is CH;
X⁷ is C(R⁷);
X⁸ is C(R⁸); and
R⁷ and R⁸, together with the carbon atoms to which they are attached form a 6-membered aryl or heteroaryl containing one nitrogen atom.

11. The compound of any one of claims 1-9, wherein R⁷ and R⁸, together with the carbon atoms to which they are attached form a 5-membered heteroaryl, cycloalkyl, cycloalkenyl, or heterocycloalkyl.

12. The compound of any one of claims 1-11, wherein R⁹ is monocyclic aryl, bicyclic aryl, monocyclic heteroaryl, or bicyclic heteroaryl, wherein aryl and heteroaryl are substituted with 0, 1, 2, 3, 4, or 5 substituents independently selected from the group consisting of halogen, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycloalkyl, cycloalkylalkyl, cycloalkenylalkyl, heteroarylalkyl, arylalkyl, heterocycloalkylalkyl, hydroxy, hydroxyalkyl, alkoxy, alkylthio, alkoxyalkyl, alkylene, aryloxy, arylthio, phenoxy, benzyloxy, amino, alkylamino, dialkylamino, acylamino, aminoalkyl, arylamino, diarylamino, sulfonylamino, sulfinylamino, sulfonyl, alkylsulfonyl, arylsulfonyl, aminosulfonyl, sulfinyl, —COOH, ketone, alkoxycarbonyl, aryloxycarbonyl, amide, carbamate, acyl, boronic acid, and boronic ester.

13. The compound of any one of claims 1-12, wherein R⁹ is 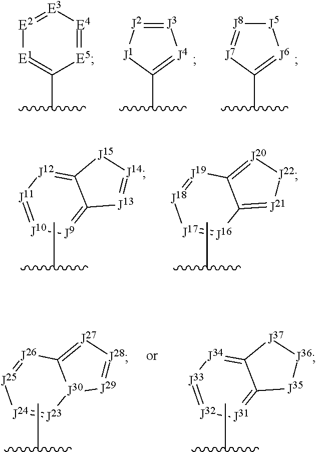
wherein
E¹-E⁵ are each independently CR²⁰ or N, wherein R²⁰, at each occurrence, is independently selected from the group consisting of hydrogen, halogen, hydroxy, cyano, amino, nitro, C₁-C₆-alkyl, C₂-C₆-alkenyl, C₂-C₆-alkynyl, C₁-C₆-alkoxy, C₁-C₆-haloalkyl, C₁-C₆-haloalkoxy, C₁-C₆-alkylamino, C₁-C₆-dialkylamino, C₁-C₆-heteroalkyl, C₁-C₆-alkylsulfonyl, —COR²¹, and —B(OR²²)₂; wherein R²¹ is selected from the group consisting of hydrogen, hydroxy, C₁-C₆-alkyl, C₁-C₆-alkoxy, C₁-C₆-haloalkyl, and C₁-C₆-haloalkoxy; wherein R²², at each occurrence, is independently selected from the group consisting of hydrogen, C₁-C₆-alkyl, and C₁-C₆-haloalkyl;
J¹ and J⁵ are O, S, or NR²³, wherein R²³ is hydrogen, C₁-C₆-alkyl, or C₁-C₆-haloalkyl;
J¹⁵ and J²² are O, S, NR²⁴, or C(═O), wherein R²⁴ is hydrogen, C₁-C₆-alkyl, or C₁-C₆-haloalkyl;
J³⁰ is N;
J²-J⁴, J⁶-J¹⁴, J¹⁶-J²¹, J²³-J²⁹, and J³¹-J³⁴ are each independently CR²⁵ or N, wherein R²⁵, at each occurrence, is independently selected from the group consisting of hydrogen, halogen, hydroxy, cyano, amino, nitro, C₁-C₆-alkyl, C₂-C₆-alkenyl, C₂-C₆-alkynyl, C₁-C₆-alkoxy, C₁-C₆-haloalkyl, C₁-C₆-haloalkoxy, C₁-C₆-alkylamino, C₁-C₆-dialkylamino, and C₁-C₆-heteroalkyl;
J³⁵-J³⁷ are each independently CR²⁶R²⁷, NR²⁸, O, and C(═O), wherein R²⁶ and R²⁷, at each occurrence, are independently selected from the group consisting of hydrogen, halogen, hydroxy, cyano, amino, nitro, C₁-C₆-alkyl, C₂-C₆-alkenyl, C₂-C₆-alkynyl, C₁-C₆-alkoxy, C₁-C₆-haloalkyl, C₁-C₆-haloalkoxy, C₁-C₆-alkylamino, C₁-C₆-dialkylamino, and C₁-C₆-heteroalkyl, and wherein R²⁸ is hydrogen, C₁-C₆-alkyl, or C₁-C₆-haloalkyl; provided that J³⁶ is not 0 when one of J³⁵ or J³⁷ is O; provided that J³⁶ is not C(═O) when one of J³⁵ or J³⁷ is C(═O);
provided that one of J⁹-J¹⁴ is C where the R⁹ attaches to the parent molecular formula;
provided that one of J¹⁶-J²¹ is C where the R⁹ attaches to the parent molecular formula; provided that one of J²³-J²⁹ is C where the R⁹ attaches to the parent molecular formula; and provided that one of J³¹-J³⁴ is C where the R⁹ attaches to the parent molecular formula.

14. The compound of any one of claims 1-13, wherein R⁹ is selected from the group consisting of:

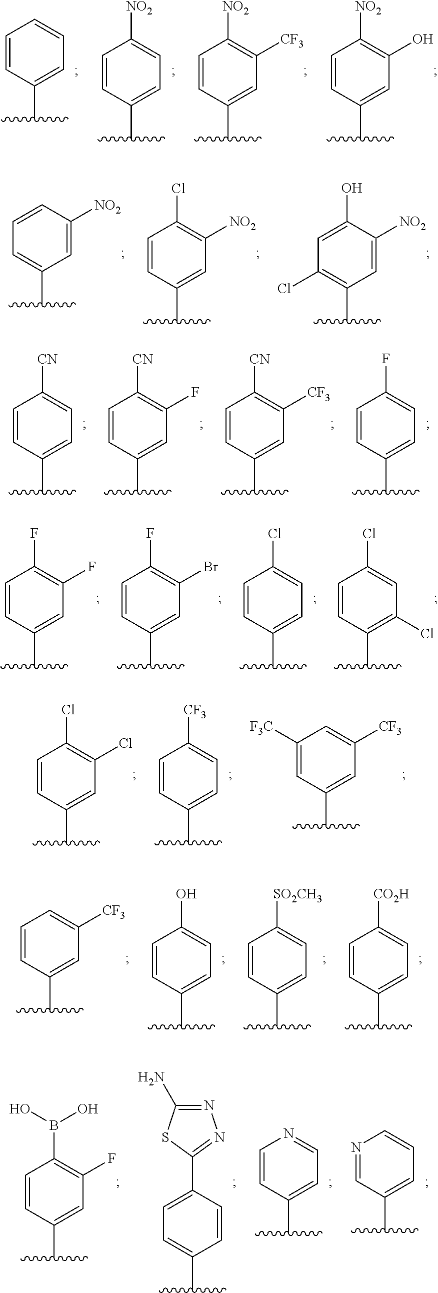

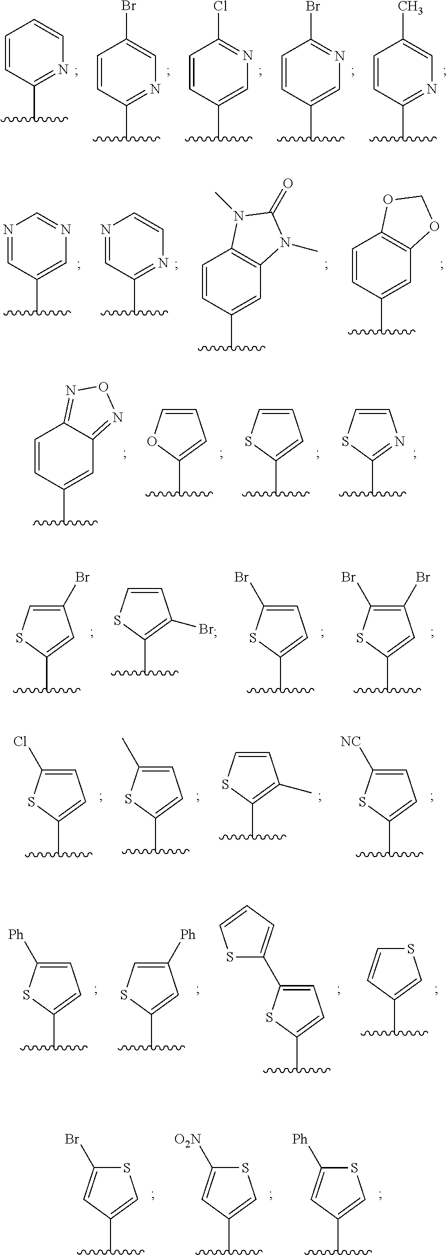

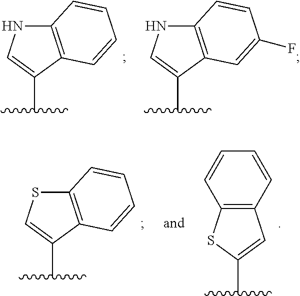

15. The compound of any one of claims 1-14, wherein R¹⁰ is hydrogen.

16. The compound of any one of claims 1-15, wherein X³ is N—R^{3c}.

17. The compound of any one of claims 1-16, wherein X³ is N—CH₃.

18. The compound of any one of claims 1-17, wherein X¹ is S(O)₂.

19. The compound of any one of claims 1-18, wherein X⁶ is C(R⁶), and R⁶ is

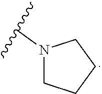 [SMILES: *N1CCCC1]

20. The compound of any one of claims 1-19, having formula: 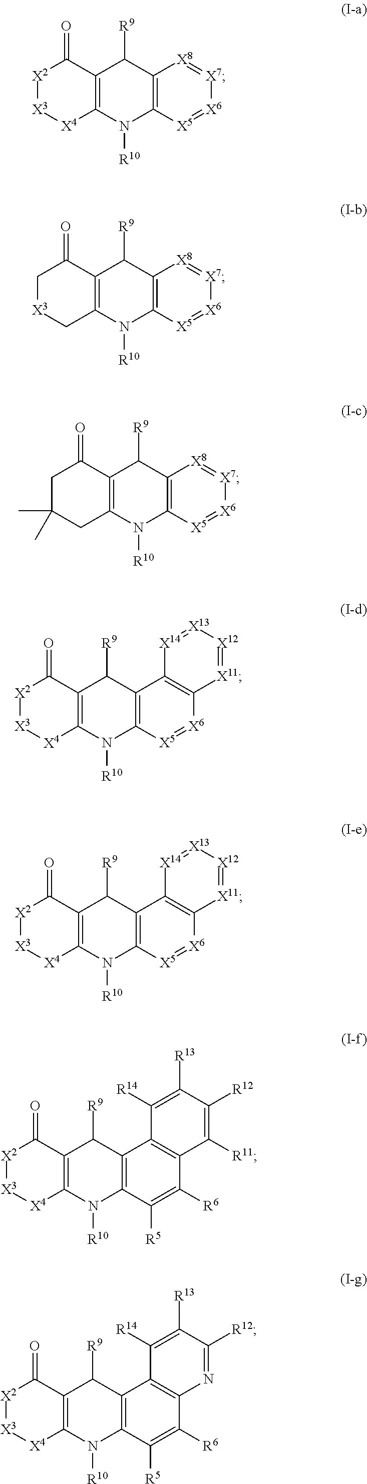 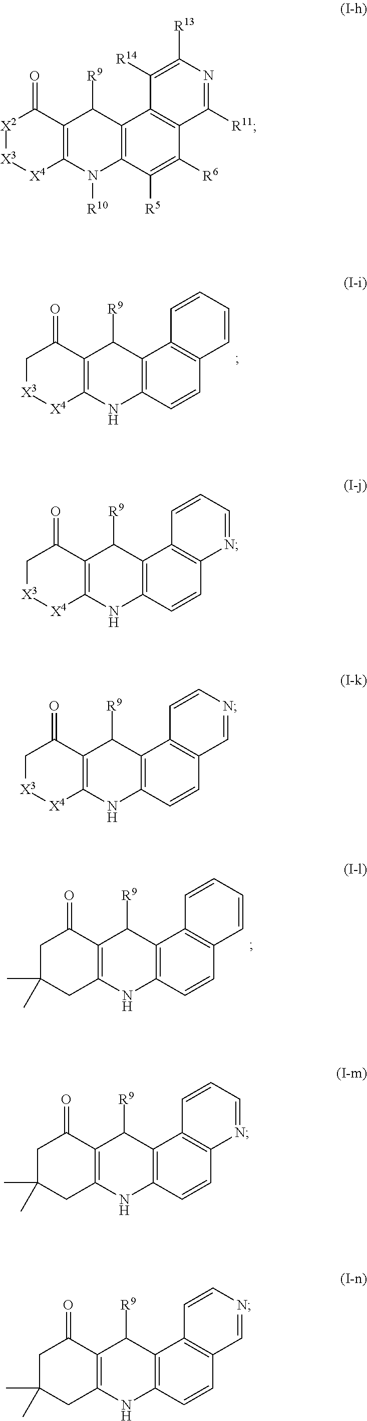 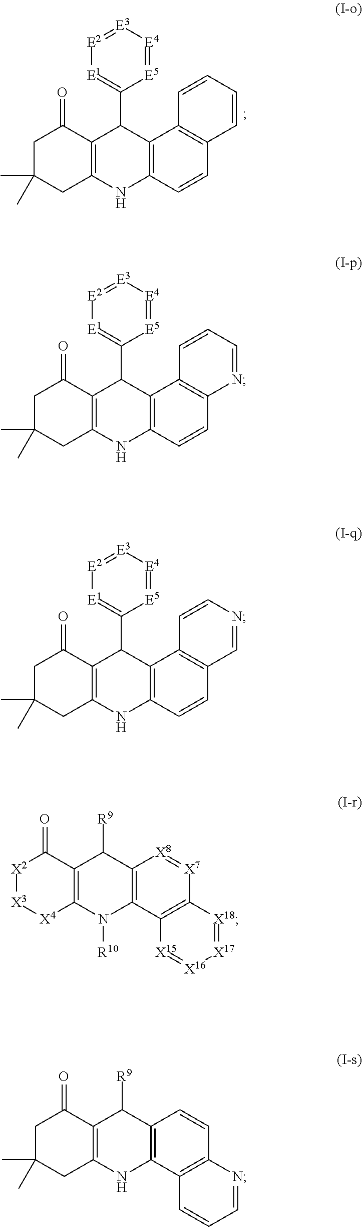 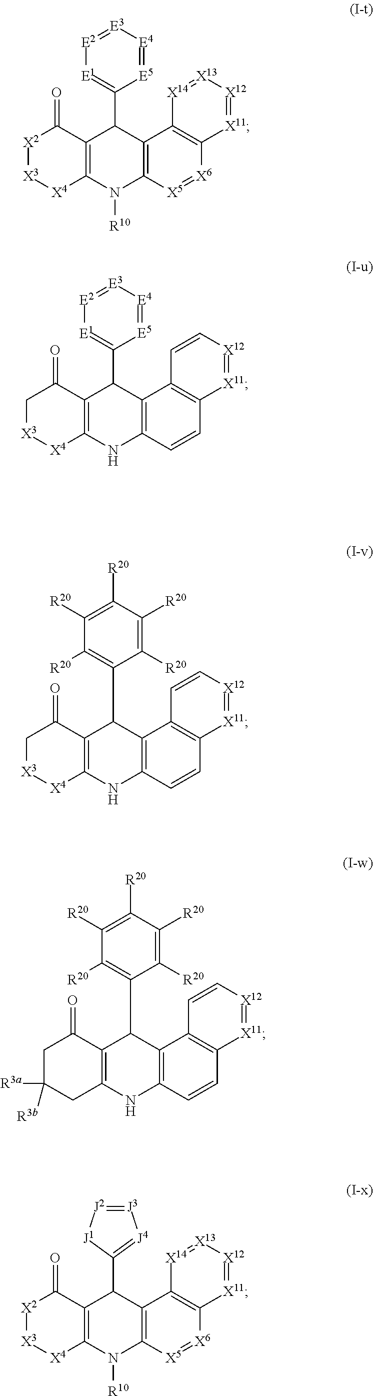 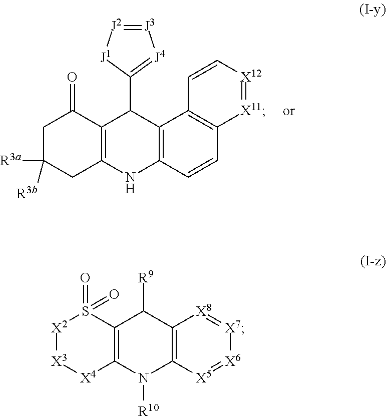
wherein
X¹¹ is C(R¹¹) or N; X¹² is C(R¹²) or N; X¹³ is C(R¹³) or N; X¹⁴ is C(R¹⁴) or N;
X¹⁵ is C(R¹⁵) or N; X¹⁶ is C(R¹⁶) or N; X¹⁷ is C(R¹⁷) or N; X¹⁸ is C(R¹⁸) or N;
R¹¹, R¹², R¹³, R¹⁴, R¹⁵, R¹⁶, R¹⁷, and R¹⁸ are each independently selected from the group consisting of hydrogen, halogen, alkyl, alkenyl, alkynyl, aryl, heteroaryl, cycloalkyl, and heterocycloalkyl;
E¹-E⁵ are each independently CR²⁰ or N, wherein R²⁰, at each occurrence, is independently selected from the group consisting of hydrogen, halogen, hydroxy, cyano, amino, nitro, C₁-C₆-alkyl, C₂-C₆-alkenyl, C₂-C₆-alkynyl, C₁-C₆-alkoxy, C₁-C₆-haloalkyl, C₁-C₆-haloalkoxy, C₁-C₆-alkylamino, C₁-C₆-dialkylamino, C₁-C₆-heteroalkyl, C₁-C₆-alkylsulfonyl, —COR²¹, and —B(OR²²)₂; wherein R²¹ is selected from the group consisting of hydrogen, hydroxy, C₁-C₆-alkyl, C₁-C₆-alkoxy, C₁-C₆-haloalkyl, and C₁-C₆-haloalkoxy; wherein R²², at each occurrence, is independently selected from the group consisting of hydrogen, C₁-C₆-alkyl, and C₁-C₆-haloalkyl;
J¹ is O, S, or NR²³, wherein R²³ is hydrogen, C₁-C₆-alkyl, or C₁-C₆-haloalkyl; and
J²-J⁴ are each independently CR²⁵ or N, wherein R²⁵, at each occurrence, is independently selected from the group consisting of hydrogen, halogen, hydroxy, cyano, amino, nitro, C₁-C₆-alkyl, C₂-C₆-alkenyl, C₂-C₆-alkynyl, C₁-C₆-alkoxy, C₁-C₆-haloalkyl, C₁-C₆-haloalkoxy, C₁-C₆-alkylamino, C₁-C₆-dialkylamino, and C₁-C₆-heteroalkyl.

21. A compound selected from the group consisting of a compound according to Table 1, or a pharmaceutically acceptable salt thereof.

22. A pharmaceutical composition comprising a therapeutically effective amount of a compound of any one of claims 1-21, or a pharmaceutically acceptable salt thereof, and a pharmaceutically acceptable carrier.

23. A method for treating a cancer, comprising administering to a mammal a therapeutically effective amount of a compound of any one of claims 1-21, or pharmaceutically acceptable salt thereof.

24. The method of claim 23, wherein the cancer is hormone refractory prostate cancer.

25. A method for modulating androgen receptor activity in a mammal, comprising administering to a mammal a therapeutically effective amount a compound of any one of claims 1-21, or pharmaceutically acceptable salt thereof.

26. The method of claim 25, wherein the mammal has hormone refractory prostate cancer.
